(19) United States
(12) Patent Application Publication
(10) Pub. No.: US 2023/0392118 A1
(43) Pub. Date: Dec. 7, 2023

(54) GENERATION OF CD4+ EFFECTOR AND REGULATORY T CELLS FROM HUMAN PLURIPOTENT STEM CELLS

(71) Applicant: Sangamo Therapeutics, Inc., Brisbane, CA (US)

(72) Inventors: Helen Fong, Oakland, CA (US); Anthony Conway, Emeryville, CA (US); Matthew C. Mendel, Richmond, CA (US)

(73) Assignee: Sangamo Therapeutics, Inc., Brisbane, CA (US)

(21) Appl. No.: 18/249,418

(22) PCT Filed: Oct. 28, 2021
(86) PCT No.: PCT/US2021/057152

§ 371 Date: Apr. 18, 2023

Related U.S. Application Data
(60) Provisional application No. 63/106,591, filed on Oct. 28, 2020.

(51) Int. Cl. C12N 5/0783

(52) U.S. Cl. CPC C12N 5/0636; C12N 2501/15; C12N 2501/999; C12N 2501/2302; C12N 2501/998; C12N 2506/45

(57) ABSTRACT

Provided herein are improved methods and compositions for generating CD4-positive effector T cells and regulatory T cells and methods of use thereof.

### CROSS-REFERENCE TO RELATED APPLICATION

This application claims priority from U.S. Provisional Application 63/106,591, filed Oct. 28, 2020, the disclosure of which is incorporated herein by reference in its entirety.

### BACKGROUND OF THE INVENTION

A healthy immune system is one that is in balance. Cells involved in adaptive immunity include B and T lymphocytes. There are two general types of T lymphocytes—effector T cells (Teffs) and regulatory T cells (Tregs). There are two general types of effector T cells—T helper (Th) cells and cytotoxic T cells (CTLs). Effector T cells expressing CD4 typically function as Th cells (CD4⁺ effector T cells), whereas effector T cells expressing CD8 typically function as CTLs (CD8⁺ effector T cells). Th cells include Th1, Th2, and Th17 cells. In addition to Th cells, unconventional T cells (e.g., NK T cells, gamma/delta T cells, and mucosal-associated invariant T cells) can also express CD4. The other unique type of T lymphocytes—Tregs—are generally known to express CD4. Treg cells regulate effector T (Teff) cells and prevent excessive immune responses and autoimmunity (see, e.g., Romano et al., Front Imm. (2019) 10:43). Traditionally defined Tregs are CD4⁺ but there have also been described CD8⁺ Tregs (Yu et al., Oncology Letters (2018) 15:6).

Teff cells play a central role in cellular-mediated immunity following antigen challenge and can include naïve, memory, stem cell memory, or terminally differentiated effector T cells. Teff cells differentiate from a naïve T cell that has experienced antigen and is performing effector functions (e.g., secreting cytokines or factors to promote a “helper” and/or cellular immune response). There can be memory or stem cell memory T helper cells as well, which can also then subsequently re-differentiate into helper T effector cells to perform effector functions.

Some Tregs are generated in the thymus; they are known as natural Treg (nTreg) or thymic Treg (tTreg). Other Tregs are generated in the periphery following antigen encounter or in cell culture, and are known as induced Tregs (iTreg) or adaptive Tregs. Tregs actively control the proliferation and activation of other immune cells, including inducing tolerance, through cell-to-cell contact involving specific cell surface receptors and the secretion of inhibitory cytokines such as IL-10, TGF-β and IL-35 (Dominguez-Villar and Hafler., Nat Immunol. (2018) 19:665-73). Failure of tolerance can lead to autoimmunity and chronic inflammation. Loss of tolerance can be caused by defects in Treg functions or insufficient Treg numbers, or by unresponsive or over-activated Teff (Sadlon et al., Clin Transl Imm. (2018) 7:e1011).

In recent years, there has been much interest in the use of Tregs to treat diseases. Several approaches, including adoptive cell therapy, have been explored to boost Treg numbers and functions in order to treat autoimmune diseases. Treg transfer (delivering an activated and expanded population of Tregs) has been tested in patients with autoimmune diseases such as type I diabetes, cutaneous lupus erythematosus, and Crohn's disease, and in organ transplantation (Dominguez-Villar, supra; Safinia et al., Front Immunol. (2018) 9:354).

CD4⁺ Teff cells have also garnered increasing interest in adoptive cell therapy. CD4⁺ Teffs are known to enhance functionality of CD8⁺ CTLs by optimizing the magnitude and quality of the CTLs' response during anti-tumor or anti-pathogen (e.g., anti-viral) priming through specific dendritic cells and other antigen-presenting cells (APCs) (Borst et al., Nat Rev Immunol. (2018) 18:635-47). CD4⁺ Teff have also been shown to be therapeutically useful in disease models of HIV (Maldini et al., Mol Ther. (2020) 28(7):1585-99). T follicular helper cells (Tfh) are an additional subset of CD4⁺ Teffs that have shown therapeutic promise by enhancing B cell response (Kamphorst et al., Immunotherapy (2013) 5(9):975-98).

Currently, the only sources of CD4⁺ Teff and Tregs for cell therapies are adult or adolescent primary blood (e.g., whole blood or apheresis products) and tissue (e.g., thymus). Isolation of CD4⁺ Teff and Tregs from these sources is invasive and time-consuming, and yields only small numbers of cells, particularly of Tregs. Further, CD4⁺ Teff and Tregs obtained from these sources are polyclonal in nature and can introduce variability in their potential immunosuppressive response. There also is evidence that simply increasing the number of Tregs may not be sufficient to control disease (McGovern et al., Front Immunol. (2017) 8:1517).

Thus, there remains a need for efficiently obtaining antigen-specific CD4⁺ Teff and Treg cells, especially genetically engineered ones, in large numbers.

### SUMMARY OF THE INVENTION

The present disclosure provides a method of obtaining a population of cells enriched for CD4⁺ T cells, comprising providing a starting population of CD4⁺CD8⁺ immature T cells, culturing the population of cells in a medium comprising phorbol 12-myristate 13-acetate (PMA) and ionomycin (I), thereby obtaining a population of cells enriched for CD4 single positive T cells. In some embodiments, the CD4 single positive T cells are immature CD4⁺ T cells, optionally wherein the T cells express ThPOK. In certain embodiments, the immature CD4⁺ T cells are effector T (Teff) cells, optionally wherein the Teff cells are CD25^{low}.

In some embodiments, the culture medium comprises about 0.00625 to about 0.1 μg/ml PMA and about 0.125 to about 2 μg/ml of ionomycin. In further embodiments, the culture medium comprises 0.00625 μg/ml PMA and 0.125 μg/ml of ionomycin. In some embodiments, the weight ratio of PMA to ionomycin in the culture medium is about 1:10 to 1:1000 (e.g., 1:20, 1:50, 1:100, 1:200 1:250, or 1:500). In certain embodiments, the ratio is 1:20.

In some embodiments, the starting population of CD4⁺CD8⁺ T cells is cultured in the medium for about one to five days.

In some embodiments, the present disclosure provides a method of obtaining a population of cells enriched for CD4⁺ T cells (e.g., human cells), comprising providing a starting population of CD4⁺CD8⁺ T cells, culturing the population of cells in a medium comprising phorbol 12-myristate 13-acetate (PMA) and ionomycin (I), thereby obtaining a population of cells enriched for CD4 single positive T cells, culturing the CD4 single positive T cells in a second medium (for, e.g., about 5-10 days) comprising IL-2, an anti-CD2 antibody, an anti-CD3 antibody, and an anti-CD28 antibody, thereby obtaining a population of cells enriched for CD4⁺ regulatory T (Treg) cells. In further embodiments, the second medium further comprises TGF-β and all trans-retinoic acid (ATRA). The second medium may also be composed of a T cell-specific medium with the addition of IL-2; in further embodiments, the second medium is composed a T-cell specific medium with the addition of one or more of TGF-β, ATRA, IL-2, an anti-CD2 antibody, an anti-CD3 antibody, and an anti-CD28 antibody.

In some embodiments, the CD4 single positive Teff or CD4 single positive and CD25 positive Treg cells may be isolated from the tissue culture by, e.g., fluorescence-activated cell sorting (FACS) or magnetic-activated cell sorting (MACS). In some embodiments, the CD4 single positive Teff or CD4⁺CD25⁺CD127^{low} Treg cells may be isolated from the tissue culture by FACS. In some embodiments, CD4⁺CD25^{high}CD127^{low} and CD4⁺CD25^{low} CD127^{low} Treg cells may be isolated from the tissue culture by FACS.

In certain embodiments; the population of CD4⁺CD8⁺ T cells are derived from human induced pluripotent stem cells (iPSCs), e.g., iPSCs reprogrammed from T cells.

In some embodiments, the iPSCs comprise a heterologous sequence in the genome, wherein the heterologous sequence comprises a transgene encoding a lineage commitment factor (e.g., FOXP3, Helios, or ThPOK), and wherein the lineage commitment factor (i) promotes the differentiation of the iPSCs to CD4⁺ Teff cells or (ii) promotes the differentiation of the iPSCs to CD4⁺ Treg cells or promotes the maintenance of the phenotype of the CD4⁺ Treg cells. In further embodiments, the heterologous sequence is integrated into a T cell specific gene locus (e.g., a T cell receptor alpha constant or TRAC gene locus) such that expression of the transgene is under the control of transcription-regulatory elements in the gene locus. In some embodiments, the heterologous sequence is integrated into exon 1, 2, or 3 of the TRAC gene locus.

In further embodiments, the transgene comprises a coding sequence for an additional polypeptide (e.g., another lineage commitment factor, a therapeutic protein, or an antigen receptor such as a chimeric antigen receptor), wherein the coding sequence for the lineage commitment factor and the coding sequence for the additional polypeptide are separated by an in-frame coding sequence for a self-cleaving peptide or by an internal ribosome entry site (IRES).

In some embodiments, the heterologous sequence is integrated into an exon in the T cell specific gene locus and comprises an internal ribosome entry site (IRES) immediately upstream of the transgene; or a second coding sequence for a self-cleaving peptide immediately upstream of and in-frame with the transgene.

In certain embodiments, the heterologous sequence further comprises, immediately upstream of the IRES or the second coding sequence for a self-cleaving peptide, a nucleotide sequence comprising all the exonic sequences of the T cell specific gene locus that are downstream of the integration site, such that the T cell specific gene locus remains able to express an intact T cell specific gene product.

In certain embodiments, the transgene comprises a coding sequence for FOXP3, a coding sequence for Helios, and/or a coding sequence of ThPOK, wherein these coding sequences are in-frame and are separated by an in-frame coding sequence for a self-cleaving peptide.

In some embodiments, the starting population of cells comprise a null mutation in a gene selected from a Class II major histocompatibility complex transactivator (CIITA) gene, an HLA Class I or II gene, a transporter associated with antigen processing, a minor histocompatibility antigen gene, and a 132 microglobulin (B2M) gene.

In some embodiments, the starting population of cells comprise a suicide gene optionally selected from a HSV-TK gene, a cytosine deaminase gene, a nitroreductase gene, a cytochrome P450 gene, or a caspase-9 gene.

In some aspects, the present disclosure also provides a population of cells enriched for CD4 single positive cells or a population of cells enriched for CD4⁺ Teff cells obtained by the present methods. In related aspects, the present disclosure provides a method of treating cancer, an infectious disease, an allergy, asthma, or an autoimmune or inflammatory disease in a patient in need thereof, comprising administering a population of cells enriched for CD4⁺ Teff cells obtained herein; the use of a population of cells enriched for CD4⁺ Teff cells obtained herein for the manufacture of a medicament in the treatment method; and a population of cells enriched for CD4⁺ Teff cells obtained herein for use in the treatment method.

In some aspects, the present disclosure provides a population of cells enriched for CD4⁺ Treg cells obtained herein. In related aspects, the present disclosure provides a method of treating a patient in need of immunosuppression, comprising administering the population of cells enriched for CD4⁺ Treg cells obtained by the present methods; the use of the population of cells enriched for CD4⁺ Treg cells obtained by the present methods in the manufacture of a medicament in treating a patient in need of immunosuppression; and a population of cells enriched for CD4⁺ Treg cells obtained by the present methods for use in treating a patient in need of immunosuppression. In some embodiments, the patient has an autoimmune disease; or has received or will receive tissue transplantation.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a diagram depicting the process used to generate hematopoietic stem and progenitor cells (HSPCs), lymphoid progenitor cells, double positive (DP) T cells, CD4 single positive (CD4sp) T cells, CD4 Th cells and Tregs from iPSCs. Lymphoid progenitor cells: CD5⁺ CD7⁺. Double positive: CD4⁺CD8⁺. Single positive: CD4⁺ or CD8⁺.

FIG. 2 is a diagram depicting the process for generating Tregs from iPSCs cultured in the presence of a T cell-specific medium (“T Cell Media”) with IL-2.

FIGS. 3 and 4 are panels of flow cytometry plots evaluating the ability of PMA and ionomycin (PMAI) to promote differentiation of DP T cells to CD4sp cells when added at day (FIG. 3) or day 42 (FIG. 4). PMAI was added at various concentrations, with the lower concentrations (0.125×, 0.25×, 0.50×, 1×, or 2×) producing more CD4sp cells. The components of the serially diluted PMAI are shown in Table A.

FIG. 5 is a panel of graphs demonstrating the re-expression of the CD4 molecule in PMAI-induced CD4sp T cells. Cells were treated with increasing concentrations of the pronase enzyme (no treatment, 0.02%, 0.04%, 0.08%, 0.1%) to strip off CD4 and incubated at either 4° C. or 37° C. to prevent or allow CD4 re-expression, respectively. CD4 MFI (mean fluorescent intensity) was measured after 1 or 2 days post-pronase treatment. The bar graph (left) shows an increase of CD4 MFI after two days of culture at 37° C. over cells at 4° C. and the flow plots (right) demonstrate CD4 re-expression at the 0.1% concentration compared to cells at 4° C.

FIGS. 6A and 6B are panels of flow cytometry histograms showing the expression of Treg markers in PMAI-induced CD4sp T cells that were treated with TGF-1131, ATRA, IL-2 and CD3/CD28/CD2 T cell activators. Various concentrations of PMAI were added at day 35 (FIG. 6A) or day 42 (FIG. 6B) to induce CD4sp T cells. Expression of FOXP3, Helios, CTLA-4, GARP, and LAP was examined.

FIG. 7 is a panel of flow cytometry histograms showing the expression of Treg markers in activated PMAI-induced and TGF-β1-treated CD4sp T cells (iPSC-Tregs). Expression of FOXP3, Helios, CTLA-4, GARP, and LAP was examined.

FIG. 8 is a panel of flow cytometry histograms showing the suppressive ability of PMAI-induced and TGF-β1-treated iPSC-Tregs on the proliferation of responder T cells. Tresp: responder T cells.

FIG. 9 is a panel of flow cytometry plots and histograms showing the expression of Treg markers in PMAI-induced CD4sp T cells that were treated with TGF-β, ATRA, IL-2, and CD3/CD28/CD2 T cell activators. CD4sp T cells were differentiated in either 100% StemSpan™ T Cell Maturation media (Maturation) or a 50% mixture of Maturation and T cell-specific media with an additional supplementation of IL-2. CD4sp T cells differentiated in a 50% mixture of Maturation and T cell-specific media with supplemental IL-2 and CD3/CD28/CD2 activators but no TGFB and ATRA was used as a control. Expression of CD4, CD8, CD25, CD127, and FOXP3 was examined. Expression of FOXP3 was examined in CD4spCD127^{low}CD25^{high} and CD4spCD127^{low}CD25^{low} cells.

FIG. 10 is a panel of flow cytometry graphs depicting the sorting strategy used to sort CD25 high and CD25 low iPSC-Tregs. Expression of CD4, CD8, CD25, and CD127 was determined in order to position gates for sorting.

FIG. 11 is a panel of flow cytometry plots showing the expression of Treg markers in unactivated and activated CD25^{high} and CD25^{low} sorted iPSC-Tregs. Expression of CD4, CD8, CD25, FOXP3, CD69, and GARP was examined.

FIG. 12 is a panel of flow cytometry graphs showing the expression of Treg markers in unactivated and activated CD25^{high} and CD25^{low} sorted iPSC-Tregs. Expression of FOXP3, Helios, CTLA-4, and LAP was examined.

FIG. 13 is a panel of graphs showing the suppressive ability of sorted iPSC-Tregs or iPSC-CD4sp T cells on responder T proliferation. iPSC-Tregs or iPSC-CD4sp T cells were cocultured with responder T cells in increasing concentrations up to 1:1 (Treg: responder T) (n=3).

FIG. 14 depicts the percentage of cells demethylated at FOXP3 Treg-specific demethylated region (TSDR) in iPSC-derived Tregs from two different clones. Methylation was determined in iPSC-Tregs after treatment with PMAI and TGF-β1 (Panel A), in CD4sp cells after treatment with 0.125×PMAI only, and in primary Treg and responder T cell (Tresp) controls (Panel B). Expression of FOXP3 was also examined by flow cytometry in PMAI and TGF-β1-treated cells (Panel C).

FIG. 15 depicts the percentage of cells demethylated at FOXP3 TSDR in iPSC-derived Tregs before (pre-Ficoll) and after (post-Ficoll) removal of dead cells and after Treg enrichment in the target cells (Treg) and non-target cells (flowthrough) (Panel A). Panel B shows flow cytometry plots demonstrating the expression of FOXP3 in iPSC-Tregs after dead cell removal and Treg enrichment.

FIG. 16 depicts the percentage of cells demethylated at FOXP3 Treg-specific demethylated region (TSDR) in sorted iPSC-Tregs. Methylation was determined in CD25^{high}, CD25^{low} iPSC-Tregs, and in bulk unsorted population. Primary Tregs and Tresp were used as controls.

FIGS. 17A-C are panels of flow cytometry plots depicting the ability of various types of T cell stimulation to generate Tregs. Markers for mature CD4⁺ T cells (FIG. 17A), Tregs (FIG. 17B), and naïve Tregs (FIG. 17C) were examined.

FIG. 18 is a panel of flow cytometry plots depicting the ability of various concentrations of PMAI to generate CD4sp T cells from DP T cells followed by generation of iPSC-Tregs after TGF-β1 and ATRA treatment.

FIG. 19 is a schematic diagram depicting a genome editing approach to integrating a transgene encoding one or more Treg commitment (or induction) factors (“TFs”) into exon 2 of the human TRAC gene. A zinc finger nuclease (ZFN) produced from an introduced mRNA makes a double-stranded break at a specific site (lightning bolt) in exon 2. The donor sequence, introduced by an adeno-associated virus (AAV) 6 vector, contains, from 5′ to 3′: homology region 1; a coding sequence for self-cleaving peptide T2A; a coding sequence for a fusion of a first TF, self-cleaving peptide P2A, a second TF2, self-cleaving peptide E2A and a third TF3; a poly-adenylation (polyA) signal sequence; and homology region 2. The homology regions are homologous to the genomic regions flanking the ZFN cleavage site. The TRAC exon 2 portion upstream of the integration site, the T2A coding sequence, and the TF coding sequence(s) are in-frame with each other. Under this approach, expression of the TRAC protein is knocked out as a result of the transgene integration. Expression of the integrated sequences is regulated by the endogenous TCR alpha chain promoter.

FIG. 20 is a schematic diagram depicting a genome editing approach similar to the one depicted in FIG. 19, but here, the heterologous sequence comprises a partial TRAC cDNA encompassing the TRAC exonic sequences downstream of the integration site (i.e., the exon 2 sequence 3′ to the integration site and the exon 3 sequence). This partial TRAC cDNA is placed immediately upstream of, and in-frame, with the T2A coding sequence, such that the engineered locus expresses an intact TCR alpha chain and TF(s) under the endogenous TCR alpha chain promoter.

FIG. 21 is a schematic diagram depicting yet another genome editing approach to integrating a transgene encoding one or more commitment factors. In this approach, the transgene is integrated into a genomic safe harbor. In this figure, the transgene is inserted into intron 1 of the human AAVS1 gene locus and linked operably to a doxycycline (Dox) inducible promoter. SA: splice acceptor. 2A: coding sequence for self-cleaving peptide 2A. PuroR: puromycin-resistant gene. TI: targeted integration.

FIG. 22 is a panel of graphs showing data generated from cells edited using the schematic outlined in FIG. 21. The transgene encodes a green fluorescent protein (GFP). Puro: puromycin. Dox: doxycycline. Puro: puromycin.

FIG. 23 is a schematic diagram depicting a genome editing approach in which a transgene encoding one or more commitment factors is integrated into intron 1 of the human AAVS1 gene. The heterologous sequence integrated into the genome includes a CAR-encoding sequence. Once Treg differentiation is accomplished, the transgene encoding the commitment factor (placed between the two LoxP sites) is excised, leaving only the CAR expression cassette at the integration site.

FIG. 24 is a schematic diagram depicting a genome editing approach to integrating a transgene encoding one or more commitment factors into exon 2 of the human TRAC gene. In this approach, the heterologous sequence integrated into the genome includes a CAR-encoding sequence. Once Treg differentiation is accomplished, the transgene encoding the commitment factor (placed between the two LoxP sites) is excised, leaving only the CAR expression cassette at the integration site.

FIG. 25 is a schematic diagram depicting a process for reprogramming mature Tregs having a single rearranged TCR to inducible pluripotent stem cells (iPSCs). Following expansion, the iPSCs are re-differentiated back into a Treg phenotype. The TCR here targets an antigen that is not an allo-antigen.

FIG. 26 is a schematic diagram depicting a process in which an iPSC differentiates into a Treg. HSC: hematopoietic stem cell. Single positive: CD4⁺ or CD8⁺. Double positive: CD4⁺CD8⁺.

FIG. 27 is a panel of cell sorting graphs demonstrating that introduction of an antibody for the alpha unit of the IL-7 receptor (IL-7Ra) to tissue culture media skews the differentiation of iPSC-derived progenitor T cells from forming CD8 single positive cells (top left quadrants) to forming CD4 single positive cells (bottom right quadrants). The antibody was added to tissue culture media at three concentrations (low, medium, and high). This effect was shown in two separate experiments (Expt. #1 and Expt. #2).

FIG. 28 is a schematic diagram depicting multiple processes for differentiating iPSCs into Tregs. The cells are cultured on Lymphocyte Differentiation Coating Material (feeder independent) or with OP9 stromal cells or OP9-DLL1 stromal cells (OP9 cells expressing the Notch ligand, Delta-like 1) stromal cells (feeder dependent). The cells are then further cultured as depicted in FIG. 26 to promote differentiation into Tregs. In an alternative path, the three-dimensional embryonic mesodermal organoids (EMO) are formed by co-culturing iPSCs with MSS-DLL1/4 or EpCAM⁻CD56⁺ stromal cells; after hematopoietic induction of the EMO, artificial thymic organoids (ATO) are formed, which are induced to generate mature Tregs with a TCR repertoire more akin to thymically selected Tregs.

### DETAILED DESCRIPTION OF THE INVENTION

The present disclosure provides methods and compositions for promoting differentiation of stem cells, such as induced pluripotent stem cells (iPSCs), and hematopoietic progenitor cells into CD4⁺ effector and/or regulatory T cells.

In one aspect, the present disclosure provides tissue culture methods and compositions for generating CD4⁺ T cells from stem and progenitor cells (e.g., iPSCs, hematopoietic stem and progenitor cells (HSPCs), lymphoid progenitor cells, or immature progenitor T cells) by activating the protein kinase C (PKC) pathway and/or other T cell signaling pathways (e.g., TCR activation pathway) through, e.g., increased intracellular calcium flux, to such an extent as to mimic natural CD4⁺ Teff and/or Treg development within the thymus. These tissue culture methods utilize small molecules and biological factors to promote the intended developmental pathway. The present methods can generate CD4 single positive (CD4sp) T cells from iPSCs that can further mature into CD4sp effector T cells and FOXP3⁺ Treg-like cells with suppressive function.

In another aspect, the present disclosure provides genetic engineering methods and compositions for further promoting differentiation of stem and progenitor cells to CD4⁺ Teffs and Tregs. In these methods, the parental cells are genetically engineered to overexpress (i.e., express at a level higher than the cell normally would) CD4⁺ helper T cell lineage commitment factors (e.g., Gata3 and ThPOK) and/or Treg lineage commitment factors (e.g., FOXP3, Helios, Ikaros). These factors facilitate the differentiation of the engineered stem and/or progenitor cells into the intended cell types.

CD4⁺ Teff and Treg cells obtained by the present methods may be autologous or allogeneic and can be used in cell-based therapy. For example, the Teff cells may be used to treat patients in need of enhanced immunity, such as patients who have cancer or an infection (e.g., a viral infection). The Treg cells may be used to treat patients in need of induction of immune tolerance or restoration of immune homeostasis, such as patients receiving organ transplantation or allogeneic cell therapy and patients with an autoimmune disease.

The present Teff and Treg cells will have improved therapeutic efficacy because they can be monoclonal, avoiding the variability caused by polyclonality in past T cell therapies. Further, the Teff and Treg cells may be selected based on their antigen specificity. For example, Teff and Treg cells may be selected for expressing a T cell receptor (TCR) or an edited-in chimeric antigen receptor (CAR) specific for an antigen at an in vivo site where the T cells are desired such that the TCR or CAR directs the T cells to the site (e.g., a site of inflammation for Treg cells, or a tumor site for Teff cells), thereby enhancing the potency of the cells.

The present Teff and Treg cells can be derived from cell populations with self-renewal attributes and pluripotency (e.g., iPSCs) or multipotency (e.g., HSPCs, lymphoid progenitor cells, or immature progenitor T cells). Thus, compared to primary T cells, the present CD4sp Teffs and Tregs have the potential to yield relatively low-cost adoptive cell therapies for use in oncology, infectious disease, autoimmune and inflammatory disease, and other therapeutic applications, compared to primary CD4sp Teffs and Tregs.

As used herein, the terms “CD4⁺ effector T cells” and “CD4⁺ Teffs” refer to a subset of T lymphocytes marked by the phenotype CD4⁺CD25^{low}. They are a subset of CD4f T cells which do not include Tregs, which are known to express high levels of CD25.

As used herein, the terms “regulatory T cells,” “regulatory T lymphocytes,” and “Tregs” refer to a subpopulation of T cells that modulates the immune system, maintains tolerance to self-antigens, and generally suppresses or downregulates induction and proliferation of T effector cells. The Treg phenotype is in part dependent on the expression of the master transcription factor forkhead box P3 (FOXP3), which regulates the expression of a network of genes essential for immune suppressive functions (see, e.g., Fontenot et al., Nature Immunology (2003)4(4):330-6). Tregs often are marked by the phenotype of CD4⁺CD25⁺CD127^{lo}FOXP3⁺. In some embodiments, Tregs are also CD45RA⁺, CD62L^{hi}, Helios⁺, and/or GITR⁺. In particular embodiments, Tregs are marked by CD4⁺CD25⁺CD127^{lo}CD62L⁺ or CD4⁺CD45RA⁺CD25^{hi}CD127^{lo}.

I. Tissue Culture Methods for Generating CD4⁺ Teff and Treg Cells from Pluripotent and Multipotent Cells

The starting cell population for generation of CD4⁺ Teff and Treg cells are mammalian cells, such as human cells, cells from a farm animal (e.g., a cow, a pig, or a horse), and cells from a pet (e.g., a cat or a dog). The cells may be pluripotent stem cells (PSCs). Pluripotent stem cells (PSCs) can be expanded indefinitely and give rise to any cell type within the human body. PSCs represent an ideal starting source for producing large numbers of differentiated cells for therapeutic applications. PSCs include, for example, embryonic stem cells (ESCs), PSCs derived by somatic cell nuclear transfer, and induced PSCs (iPSCs). See, e.g., Iriguchi and Kaneko, Cancer Sci. (2019) 110(1):16-22 for differentiating iPSCs to T cells. As used herein, the term “embryonic stem cells” refers to pluripotent stem cells obtained from early embryos; in some embodiments, this term refers to ESCs obtained from a previously established embryonic stem cell line and excludes stem cells obtained by recent destruction of a human embryo.

In other embodiments, the starting cell populations may be multipotent cells such as mesodermal stem cells, mesenchymal stem cells, hematopoietic stem cells (e.g., those isolated from bone marrow or cord blood), or hematopoietic progenitor cells (e.g., lymphoid progenitor cells). Multipotent cells are capable of developing into more than one cell type, but are more limited in cell type potential than pluripotent cells. The multipotent cells may be derived from established cell lines or isolated from human bone marrow or umbilical cords. By way of example, the hematopoietic stem cells (HSC) may be isolated from a patient or a healthy donor following granulocyte-colony stimulating factor (G-CSF)-induced mobilization, plerixafor-induced mobilization, or a combination thereof. To isolate HSCs from the blood or bone marrow, the cells in the blood or bone marrow may be panned by antibodies that bind unwanted cells, such as antibodies to CD4 and CD8 (T cells), CD45 (B cells), GR-1 (granulocytes), and Iad (differentiated antigen-presenting cells) (see, e.g., Inaba, et al. (1992) J. Exp. Med. 176:1693-1702). HSCs can then be positively selected by antibodies to CD34.

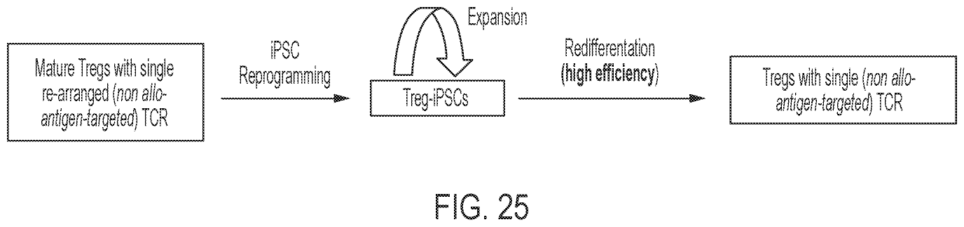

In some embodiments, the starting cell populations are human iPSCs reprogrammed from a human somatic cell, such as a fibroblast or a mature T cell such as a Treg (Takahashi et al. (2007) Cell 131(5):861-72), such as a mature Treg expressing a TCR that targets a non-allogenic antigen. See FIG. 25 and further discussions below.

The present disclosure provides methods of generating CD4⁺ Teff and Treg cells from PSCs such as induced PSCs (iPSCs). Also included in the present disclosure are methods of generating Treg cells from multipotent cells such as mesodermal progenitor cells, hematopoietic stem cells, or lymphoid progenitor cells. Multipotent cells, including multipotent stem cells and tissue progenitor cells, are more limited in their ability to differentiate into different cell types as compared to pluripotent cells.

In the present methods, pluripotent or multipotent cells can be differentiated into CD4⁺ T cells (including Teff and Treg) by activating the PKC pathway and/or other T cell signaling pathways (e.g., TCR activation pathway). Such activation mimics natural CD4⁺ Teff and/or Treg development within the thymus. Activation of these pathways may be achieved by culturing the CD4⁺CD8⁺ cells in the presence of small molecules and/or macromolecules that act as agonists of the pathways. Examples of such agonists are phorbol 12-myristate 13-acetate (PMA; for PKC activation); the ionophore ionomycin (for enhancing intracellular calcium levels and increasing calcineurin and NFAT dephosphorylation); Src kinase inhibitors (for blocking Lck activity; e.g., JNJ 10198409, A 419259 trihydrochloride, AZM 475271, and alsterpaullone, available at, e.g., Tocris); and antibodies or other proteins that promote TCR and co-receptor engagement and activation (e.g., CD3, CD28, and CD2 multimeric antibody or agonist complexes such as ImmunoCult™ Human CD3/CD28/CD2 T Cell Activator, available at StemCell Technologies). Co-culturing of the cells with MHC class II expressing cells (e.g., B cells, macrophages, and dendritic cells) also can mimic CD4⁺ TCR engagement during thymic selection.

In some embodiments, the CD4⁺CD8⁺ T cells (double positive T cells) are cultured in the presence of PMA and ionomycin to promote differentiation of the cells to CD4 single positive T cells. In particular embodiments, the weight ratio of PMA to ionomycin is about 1:10 to about 1:1000 (e.g., 1:20, 1:50, 1:100, 1:250, or 1:500). In some embodiments, the double positive cells are cultured in the presence of PMA and ionomycin for about one to five days, e.g., about 24 hours.

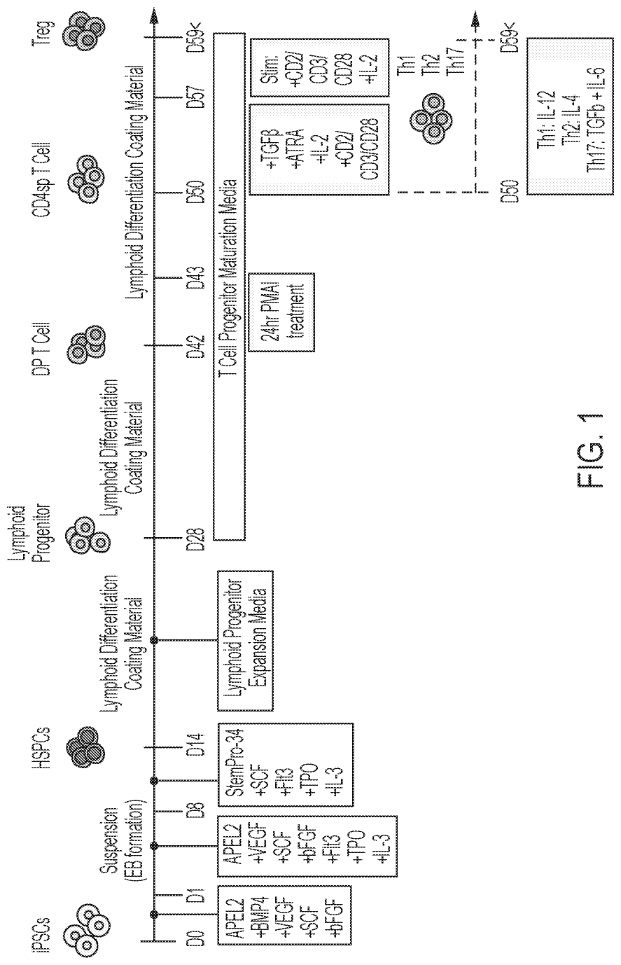

By way of example, FIG. 1 illustrates a stepwise process for generating—from iPSCs—hematopoietic stem and progenitor cells (HSPCs), lymphoid progenitor cells, double positive T cells, CD4sp Teffs and Tregs. In this exemplary process, the iPSCs may be first grown into embryoid bodies (EBs) for about 5-12 days (e.g., about 8 days) in cytokines and growth factors to direct their differentiation to the mesoderm lineage and then to HSPCs. For example, iPSCs may be cultured in a STEMdiff™ APEL2™ medium (StemCell Technologies) in the presence of about 5-20 (e.g., 10) ng/ml, BMP4, about 5-20 (e.g., 10) ng/mL VEGF, about 10-100 (e.g., 50) ng/mL SCF, about 5-20 (e.g., 10) ng/mL bFGF and about 5-20 (e.g., 10) μM Y-27632 dihydrochloride for about 1-5 days (e.g., 1 day) to promote embryoid body (EB) formation. Then the EBs may be cultured in STEMdiff™ APEL2™ in the presence of about 10-50 (e.g., 20) ng/mL VEGF, about 50-200 (e.g., 100) ng/mL SCF, about 5-20 (e.g., 10) ng/mL bFGF, about 10-50 (e.g., 20) ng/mL Flt-3, about 10-50 (e.g., 20) ng/mL TPO and about 10-100 (e.g., 40) ng/mL IL-3 for about 1 to 10 days (e.g., 7 days), and then in StemPro™-34 (Thermo Fisher) in the presence of about 50-200 (e.g., 100) ng/mL SCF, about 10-50 (e.g., 20) ng/mL Flt-3, about 10-50 (e.g., 20) ng/mL TPO and about 10-100 (e.g., 40) mg/mL IL-3 for about 8 to 14 days (e.g., 6 days) to generate a cell population enriched for HSPCs.

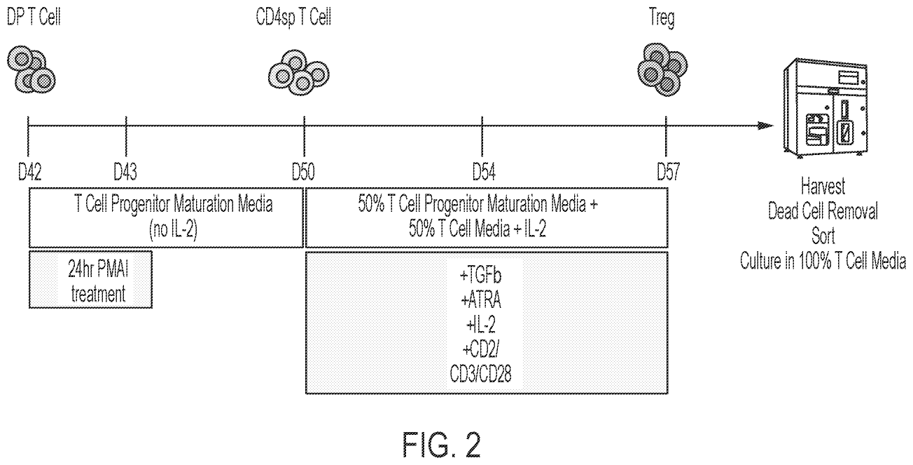

HSPCs from the EBs are further differentiated to lymphoid progenitor cells for an additional 14 days or so. The lymphoid progenitors are then differentiated to CD4⁺CD8⁺ (double positive) T cells for about 7-14 days (e.g., in a T cell progenitor maturation medium; obtainable by, e.g., mixing StemSpan™ SFEM II with T Cell Progenitor Maturation Supplement (StemCell Technologies)) at which time phorbol 12-myristate 13-acetate and ionomycin (PMAI) are added to the culture system for about 1 to 3 days (e.g., about 1 day or about 24 hours), to induce commitment to the CD4 lineage. The PMAI-inducted cells may be further cultured in the absence of PMAI for about 5-15 days (e.g., 7 days). The resulting CD4sp T cells are then further differentiated to Tregs with the addition of about 1-10 ng/mL TGF-β1, about 1-10 nM all-trans retinoic acid (ATRA), about 10-1000 U/mL IL-2, and CD3/CD28/CD2 human T cell activators (ImmunoCult™; or Dynabeads from Thermo Fisher) for about 5-10 days (e.g., about 7 days). In some embodiments, the differentiation into CD4sp T cells occurs in a medium comprising a T cell-specific medium; this culture medium can be prepared by mixing a base medium such as a T cell progenitor maturation medium and a T cell-specific medium (e.g., at a volume ratio of 1:1), and supplemented with IL-2 (e.g., about 10-1000 U/mL IL-2) (FIG. 2). T cell-specific media are culture media that promote the growth and expansion of T lymphocytes and optionally serum-free. Examples of T cell-specific media include, without limitations, OpTmizer™ (Thermo Fisher), ImmunoCult™-XF (StemCell Technologies), and X-VIVO™ 15 (Lonza). In these methods, the differentiation process from iPSC to Tregs can be entirely feeder independent. The addition of PMAI to the culture system at the double positive (CD4⁺CD8⁺) T cell stage of development is a critical step for differentiation of immature double positive T cells to CD4sp T cells and ultimately to Teffs and Tregs. The addition of IL-2 and differentiation in T cell-specific media enhance the number of overall Tregs in the population.

In some embodiments, PMA is added at 0.0001 to 0.2 (e.g., 0.0003125 to 0.1) ng/1.11 together with 0.005 to 5 (e.g., 0.00625 to 2) ng/μl ionomycin to the tissue culture of double positive T cells. In particular embodiments, lx PMAI refers to 0.05 ng/μl PMA and 1 ng/μl ionomycin in the tissue culture, and the double positive cells are cultured in the presence of 0.00625×, 0.125×, 0.25×, 0.50×, 1× or 2×PMAI, for about 24 hours. Double positive cells may also be cultured in 0.004×PMA and 0.2× lonomycin for about 24 hours. Exemplary PMAI concentrations useful for the present culture methods (e.g., 0.00625× to 2×) are shown in Table A below:

TABLE A

| PMAI Dilution | PMA (ng/μl) | Ionomycin (ng/μl) |
|---|---|---|
| cocktail stock @500X | 25 | 500 |
| 2X | 0.1 | 2 |
| 1X | 0.05 | 1 |
| 0.5X | 0.025 | 0.5 |
| 0.25X | 0.0125 | 0.25 |
| 0.125X | 0.00625 | 0.125 |
| 0.00625X | 0.0003125 | 0.00625 |
| PMA (0.004X) + I (0.2X) | 0.0002 | 0.2 |

The PMAI-inducted cells may be further cultured in the absence of PMAI for about one week, to obtain a cell population enriched for CD4sp T cells. As used herein, a cell population “enriched” for a certain cell type means that the certain cell type accounts of at least 30% (e.g., at least 35, 40, 45, 50, 55, 60, 65, 70, 75, 80, 85, or 90%) of the total cell population. In some embodiments, the PMAI-induced cell population contains at least 60-80% of CD4sp cells.

CD4sp cells may be further differentiated into CD4⁺ Teff or Treg cells. For example, as illustrated in FIGS. 1 and 2, CD4sp-enriched T cell population may be cultured in the presence of TGF-β, ATRA, IL-2, and antibodies against CD2, CD3, and CD28, and T cell-specific media for about one week to obtain Treg cells. See also Liu et al., Cell Mol Immunol. (2015) 12(5):553-7; Chen and Konkel, Eur J Immunol. (2015) 45(4):958-65.

Alternatively, the CD4sp cells may be cultured in the presence of IL-12 to obtain a Th1-enriched T cell population; in the presence of IL-4 to obtain a Th2-enriched T cell population; or in the presence of IL-6 and TGF-β to obtain a Th17-enriched T cell population.

CD4⁺ Teffs and/or Tregs may be purified from the cultured cell populations using FACS or MACS, using cell surface or intracellular markers that are specific to that cell type. For total CD4sp cells, the following markers may be used: CD3, CD4, CD8, and TCRαβ. For total Tregs, the following markers may be used: CD4, CD8, CD25, and CD127. For naïve Tregs, the following markers may be used: CD4, CD8, CD25, CD127, and CD45RA.

Purified cells may be cryopreserved or expanded for subsequent therapeutic use.

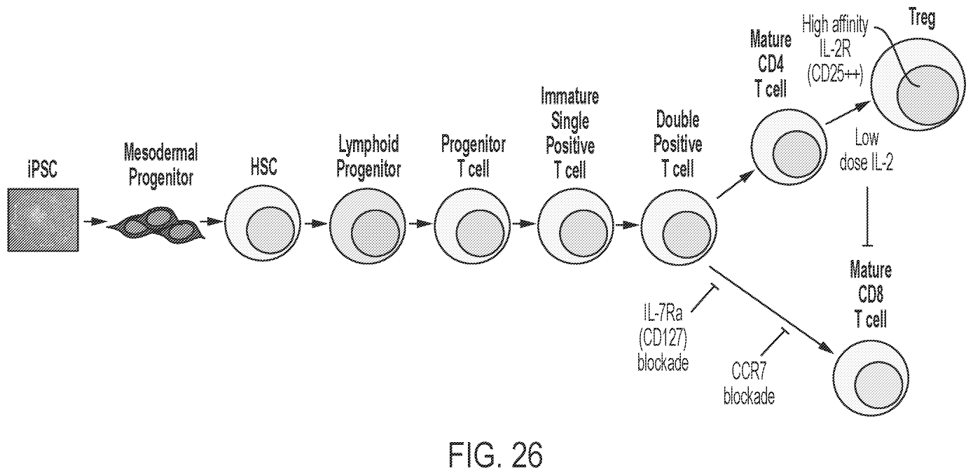

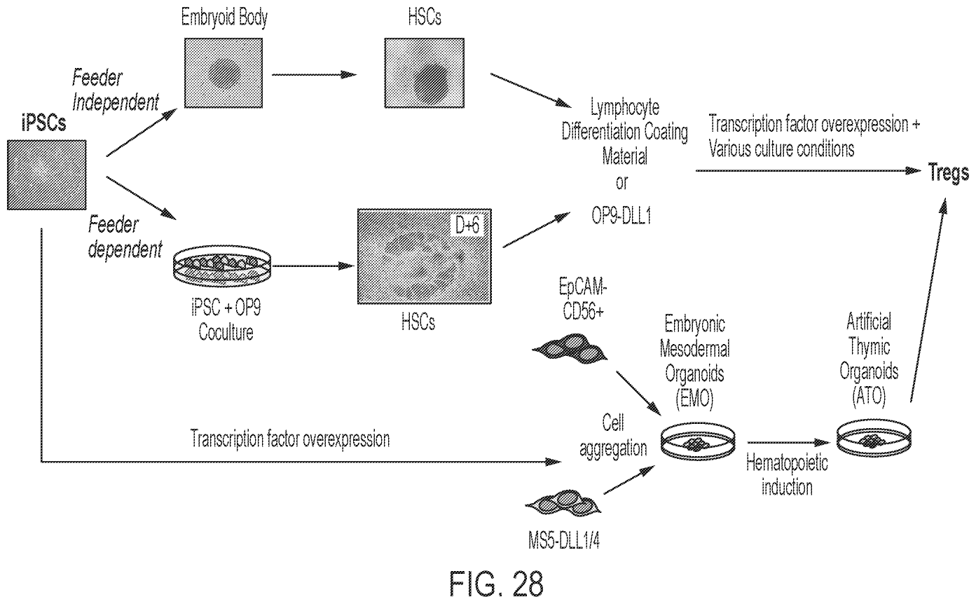

See also FIGS. 26 and 28 for processes for obtaining differentiated T cells from iPSCs.

II. Genetic Engineering Methods for Generating CD4⁺ Teff and Treg Cells from Pluripotent and Multipotent Cells

To further promote the differentiation of progenitor cells or stem cells such as iPSCs into Teffs and Tregs, the cells may be engineered to express one or more proteins that promote the lineage commitment of the progenitor or stem cells to become CD4⁺ helper T cells and ultimately Treg cells. These commitment factors may be constitutively overexpressed during the entire or part of the Treg differentiation process, or may be inducibly expressed during a specific period of the Treg differentiation process (e.g., via doxycycline-induced TetR-mediated gene expression).

In some embodiments, the commitment factors are encoded by transgene(s) randomly integrated into the genome of the stem or progenitor cells (e.g., by using a lentiviral vector, a retroviral vector, or a transposon).

Alternatively, the commitment factors are encoded by transgene(s) that are integrated into the genome of the stem or progenitor cells in a site-specific manner. For example, the transgenes are integrated at a genomic safe harbor site, or at a genomic locus of a T cell specific gene, such as the T cell receptor alpha chain constant region (i.e., T cell receptor alpha constant or TRAC) gene. In the former approach, the transgene can be optionally placed under the transcription control of a T cell specific promoter or an inducible promoter. In the latter approach, the transgene can be expressed under the control of the endogenous promoter and other transcription-regulatory elements for the T cell specific gene (e.g., the TCR alpha chain promoter). An advantage of placing the transgene under the control of a T cell specific promoter is that the transgene will only be expressed in T cells, as it is intended to be, thereby improving the clinical safety of the engineered cells.

1. Transgenes Encoding CD4⁺ Teff and Treg Commitment Factors

To further promote the differentiation of progenitor cells or stem cells such as iPSCs into Teffs and Tregs, the cells may be engineered to express one or more proteins that promote the lineage commitment of the progenitor or stem cells to become CD4⁺ helper T cells and ultimately Treg cells. In the present methods, transgenes that are introduced to the genome of the stem cells or progenitor cells to promote their differentiation to CD4⁺ Teffs are, without limitations, CD4, CTLA-4, Gata3 and ThPOK. Transgenes that can promote further differentiation into CD4⁺ Tregs may be, without limitations, those encoding one or more of CD4, CD25, FOXP3, CD4RA, CD62L, Helios, GITR, Ikaros, CTLA4, Gata3, Tox, ETS1, LEF1, RORA, TNFR2, and ThPOK. The cDNA sequences encoding these proteins are available at GenBank and other well-known gene databases. Expression of one or more of these proteins will help commit the stem or progenitor cells to the Treg fate during differentiation. In some embodiments, the transgene encodes the Treg lineage commitment factor FOXP3 and/or the CD4⁺ helper T cell lineage commitment factor ThPOK (He et al., Nature (2005) 433(7028):826-33). In some embodiments, the transgene encodes Helios, which is expressed in a subpopulation of Tregs (Thornton et al., Eur J Immunol. (2019) 49(3):398-412).

In some embodiments, the stem or progenitor cells may be engineered to overexpress commitment factors that enhance hematopoietic stem cell (HSC) multipotency (see Sugimura et al., Nature (2017) 545(7655):432-38). These factors include, without limitation, HOXA9, ERG, RORA, SOX4, LCOR, HOXA5, RUNX1, and MYB.

In some embodiments, the stem or progenitor cells may be engineered to downregulate EZHI via an engineered site-specific transcriptional repression construct (e.g. ZFP-KRAB, CRISPRi, etc.), shRNA, or siRNA to enhance HSC multipotency (see Vo et al., Nature (2018) 553(7689):506-510).

The phenotype of Tregs tends to be unstable. In some embodiments, to maintain the Treg phenotype and/or to increase expression of the Treg lineage commitment factors and the transgene in the engineered Treg cells, the cells may be cultured in tissue culture media containing rapamycin and/or a high concentration of IL-2. (See, e.g., MacDonald et al., Clin Exp Immunol. (2019) 197:11-13). Plasticity is a property inherent to nearly all types of immune cells. It appears that Treg cells can transition (“drift”) to Teff cells under inflammatory and environmental conditions (Sadlon et al., Clin Transl Imm. (2018) 7(2):e1011). Engineered monoclonal Tregs with antigen-specific moieties, such as CARs or engineered TCRs, may allow for enhanced immunomodulatory responses at the site of autoimmune activity or organ transplant.

2. Integration of Transgenes Encoding Commitment Factors

To engineer stem cells or progenitor cells genetically, a heterologous nucleotide sequence carrying a transgene of interest is introduced into the cells. The term “heterologous” here means that the sequence is inserted into a site of the genome where this sequence does not naturally occur. In some embodiments, the heterologous sequence is introduced into a genomic site that is specifically active in Treg cells. Examples of such sites are the genes encoding a T cell receptor chain (e.g., TCR alpha chain, beta chain, gamma chain, or delta chain), a CD3 chain (e.g., CD3 zeta, epsilon, delta, or gamma chain), FOXP3, Helios, CTLA4, Ikaros, TNFR2, or CD4.

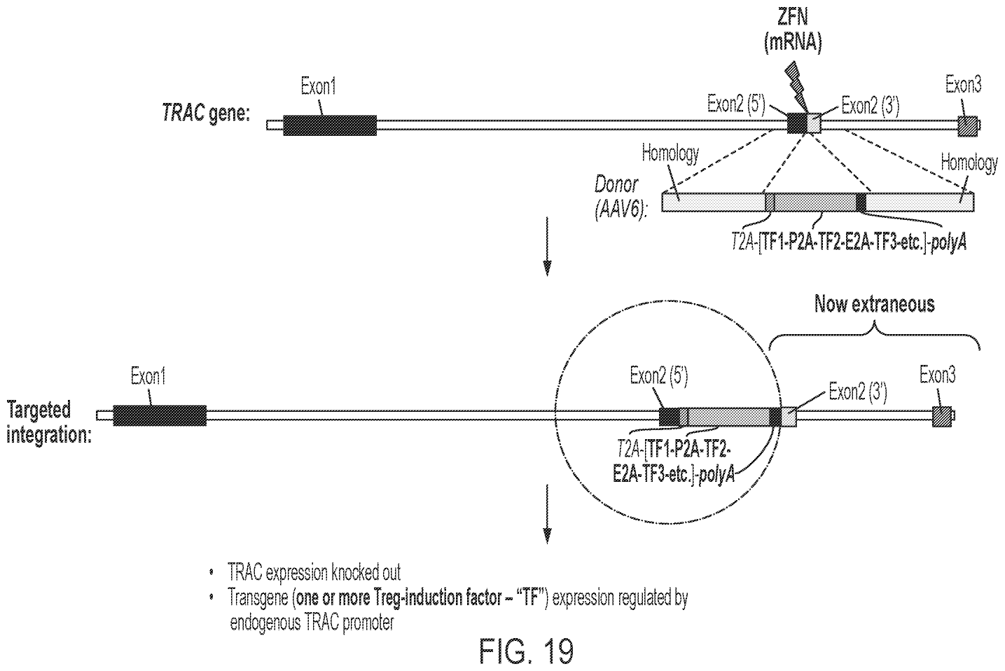

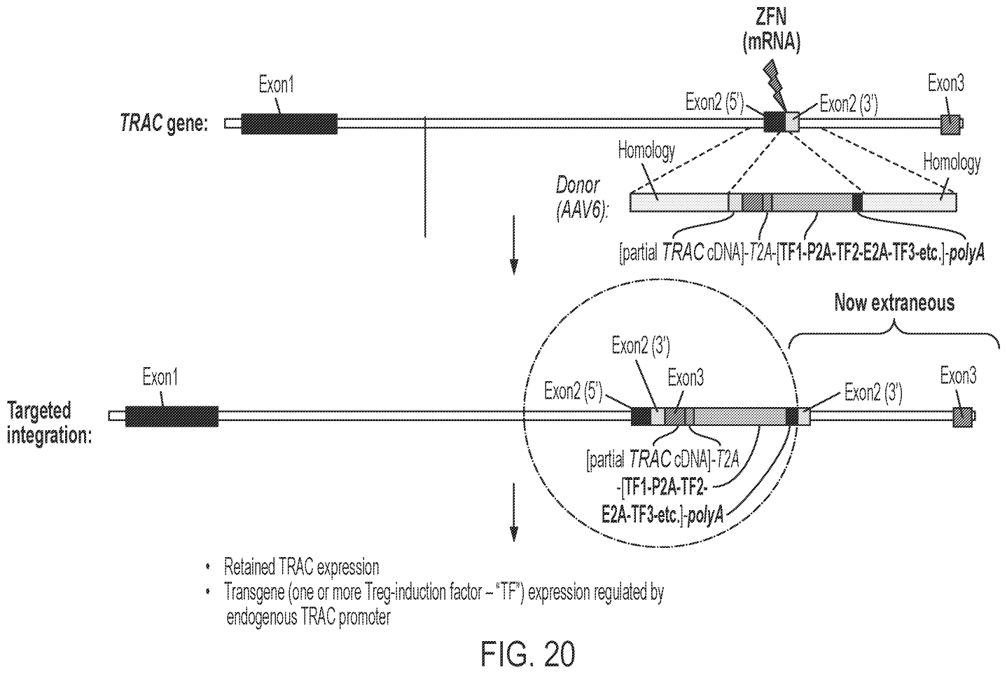

By way of example, the heterologous sequence is introduced into one or both TRAC alleles in the genome. The genomic structure of the TRAC locus is illustrated in FIGS. 19 and 20. The TRAC gene is downstream of the TCR alpha chain V and J genes. TRAC contains three exons, which are transcribed into the constant region of the TCR alpha chain. The gene sequence and the exon/intron boundaries of the human TRAC gene can be found in Genbank ID 28755 or 6955. The targeted site for integration may be, for example, in an intron (e.g., intron 1 or 2), in a region downstream of the last exon of the TRAC gene, in an exon (e.g., exon 1, 2, or 3), or at a junction between an intron and its adjacent exon.

FIGS. 19 and 20 illustrate two different approaches to targeting integration of a heterologous sequence into exon 2 of the human TRAC locus through gene editing. In both approaches, the transgene encodes a polypeptide containing one or more Treg commitment or induction factors (e.g., FOXP3), separated by a self-cleaving peptide (e.g., P2A, E2A, F2A, T2A). In some embodiments, the FOXP3 transgene is engineered to convert lysine residues, which are known to become acetylated, into arginine residues (e.g., K31R, K263R, K268R), so as to enhance Treg suppressive activity (see Kwon et al., J Immunol. (2012) 188(6):2712-21).

In the approach depicted in FIG. 19, the expression of TCR alpha chain in the engineered cell is disrupted by the insertion of the heterologous sequence. In this approach, the heterologous sequence integrated into the genome contains, from 5′ to 3′, (i) a coding sequence for self-cleaving peptide T2A (or an internal ribosome entry site (IRES) sequence), (ii) a coding sequence for the commitment factor(s), and (iii) a polyadenylation (polyA) site. Once integrated, the engineered TRAC locus will express the commitment factor(s) under the endogenous promoter, where the T2A peptide allows the removal of any TCR alpha chain sequence from the first commitment factor (i.e., any TCR variable domain sequence, as well as any constant region sequence encoded by exon 1 and the portion of exon 2 5′ to the integration site). Because the TRAC gene is disrupted, no functional TCR alpha chain can be produced in the engineered cell. Due to the inclusion of a P2A coding sequence in the transgene, the engineered locus can express all individual Treg induction factor(s) as separate polypeptides. Under this approach, the stem or progenitor cells may be further engineered to express a desired antigen-recognition receptor (e.g., a TCR or CAR targeting an antigen of interest).

In the approach depicted in FIG. 20, the heterologous sequence may contain, from 5′ to 3′, (i) the TRAC exonic sequences 3′ to the integration site (i.e., the remaining exon 2 sequence downstream of the integration site, and the entire exon 3 sequence), (ii) a coding sequence for T2A (or IRES sequence), (iii) a coding sequence for one or more commitment factors, and (iv) a polyA site. The inclusion of the TRAC exonic sequences and T2A in the heterologous sequence will allow the production of an intact TCR alpha chain. The inclusion of P2A will allow the production of the commitment factor(s) as separate polypeptides. The TCR alpha chain, the exogenously introduced commitment factor(s) are all expressed under the control of the endogenous TCR alpha chain promoter. This approach is particularly suitable for engineering iPSCs reprogrammed from a mature Treg that has already rearranged its TCR alpha and beta chain loci (see FIG. 25 and discussions below). Tregs differentiated from such genetically engineered iPSCs will retain the antigen specificity of the ancestral Treg cell. Further, retention of the TCR alpha chain expression may yield enhanced T cell and Treg differentiation since TCR signaling is integrally involved in T cell and Treg development in the thymus.

In alternative embodiments, the transgene may be integrated into a TRAC intron, rather than a TRAC exon. For example, the transgene is integrated in an intron upstream of exon 2 or exon 3. In such embodiments, the heterologous sequence carrying the transgene may contain, from 5′ to 3′, a splice acceptor (SA) sequence, the transgene encoding one or more Treg commitment factors, and a polyA site. Where the expression of a rearranged TCR alpha chain gene is desired, the heterologous sequence may contain, from 5′ to 3′, (i) an SA sequence, (ii) any exon(s) downstream of the heterologous sequence integration site, (iii) a coding sequence for a self-cleaving peptide or an IRES sequence, (iv) the transgene encoding one or more commitment factors, and (v) a polyA site. Once integrated, the SA will allow the expression of an RNA transcript encoding an intact (i.e., full-length) TCR alpha chain, the self-cleaving peptide, and the commitment factor(s). Translation of this RNA transcript will yield two (or more) separate polypeptide products—the intact TCR alpha chain and the one or more commitment factors. Examples of SA sequences are those of the TRAC exons and other SA sequences known in the art.

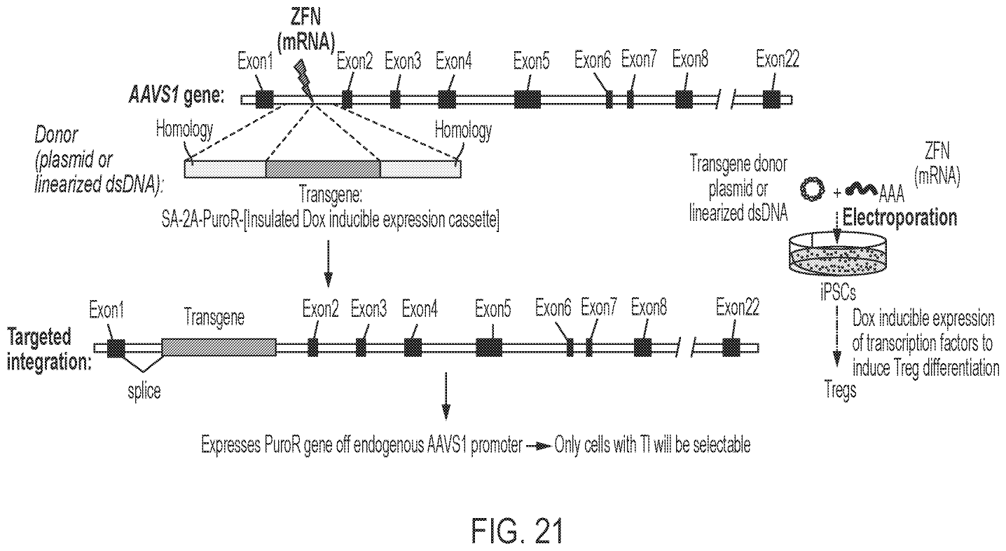

In some embodiments, the transgene is integrated into a genomic safe harbor of the engineered cells. Genomic safe harbor sites include, without limitation, the AAVS1 locus; the ROSA26 locus; the CLYBL locus; the gene loci for albumin, CCR5, and CXCR4; and the locus where the endogenous gene is knocked out in the engineered cells (e.g., the T cell receptor alpha or beta chain gene locus, the HLA gene locus, the CIITA locus, or the 02-microglobulin gene locus). FIG. 21 illustrates such an approach. In this example, the heterologous sequence is integrated into the human AAVS1 gene locus at, e.g., intron 1. Expression of the commitment factor encoding transgene is controlled by a doxycycline-inducible promoter. The doxycycline-inducible promoter may include a 5-mer repeat of the Tet-responsive element. Upon the introduction of doxycycline to tissue culture, the constitutively expressed inducible form of the tetracycline-controlled transactivator (rtTA) binds to the Tet-responsive element and initiate transcription of the commitment factor(s). A zinc finger nuclease (ZFN) produced from an introduced mRNA makes a double-stranded break at a specific site (lightning bolt) in intron 1. The donor sequence, introduced by plasmid DNA or linearized double-stranded DNA, contains, from 5′ to 3′, homology region 1, a splice acceptor (SA) to splice to AAVS1 exon 1, a coding sequence for self-cleaving peptide 2A, a coding sequence for a puromycin-resistance gene, a polyA signal sequence, a 5′ genomic insulator sequence, the doxycycline-inducible commitment factor cassette, the rtTA coding sequence driven off a CAGG promoter and followed by a polyA sequence, a 3′ genomic insulator sequence, and homology region 2. The genomic insulator sequences ensure the transgenes within them are not epigenetically silenced over the course of differentiation. The homology regions are homologous to the genomic regions flanking the ZFN cleavage site. Cells with successful targeted integration (TI) can be positively selected for by introducing puromycin into culture. Inducible expression of the Treg induction factors is useful since certain factors may be toxic during mesodermal, hematopoietic, or lymphocyte development, thus turning on the factors only during T cell development to skew differentiation towards the Treg lineage is advantageous.

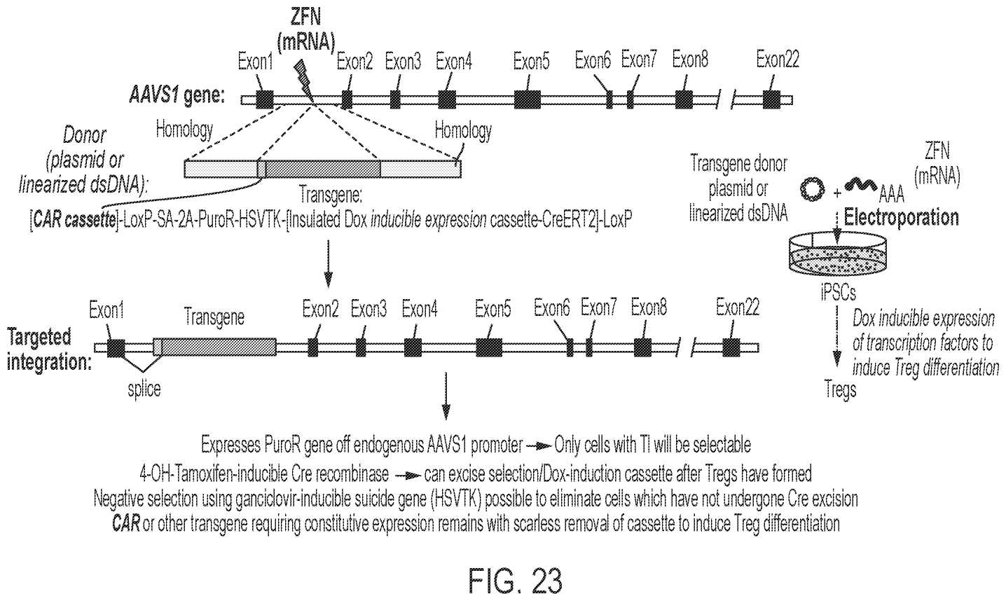

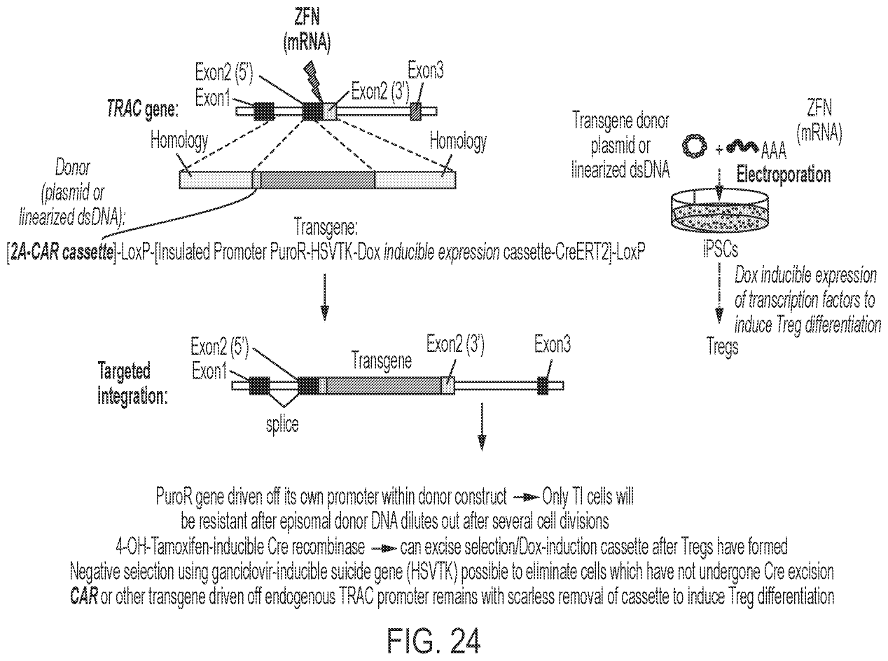

In some embodiments, the heterologous sequence contains an expression cassette for an antigen-binding receptor, such as a chimeric antigen receptor (CAR). FIGS. 23 and 24 illustrate examples of such embodiments. In FIG. 23, the heterologous sequence is introduced by plasmid DNA or linearized double-stranded DNA and contains, from 5′ to 3′, homology region 1, a CAR expression cassette (in antisense orientation to the donor) driven off its own promoter and containing a polyA site, a 5′ LoxP site, a splice acceptor to splice to AAVS1 exon 1, a coding sequence for self-cleaving peptide 2A, a coding sequence for a puromycin-resistance gene, a coding sequence for the suicide gene HSV-TK, a polyA site, a genomic insulator sequence, the doxycycline-inducible commitment factor expression cassette, the rtTA coding sequence driven off a CAGG promoter, the coding sequence for a 4-hydroxy-tamoxifen (4-OHT)-inducible form of the Cre recombinase linked to the rtTA sequence via a 2A peptide and followed by a polyA sequence, a 3′ genomic insulator sequence, a 3′ LoxP site, and homology region 2. The genomic insulator sequences ensure the transgenes within them are not epigenetically silenced over the course of differentiation. The homology regions are homologous to the genomic regions flanking the ZFN cleavage site. Cells with successful targeted integration can be positively selected for by introducing puromycin into the tissue culture. The constitutively expressed 4-OHT-inducible Cre allows for excision of the entire cassette between the LoxP sites after addition of 4-OHT to culture. Cells that have not undergone recombinase-mediated excision will still express HSV-TK and thus can be negatively selected (eliminated) by adding ganciclovir (GCV) into tissue culture. GCV will result in cell death of any cell expressing HSV-TK. This system allows for completely scarless removal of the Treg induction cassette while leaving the CAR cassette integrated to allow for targeted immunosuppression in the engineered Tregs.

FIG. 24 illustrates expression of CAR from the engineered TRAC gene. In this example, the heterologous sequence, introduced by plasmid DNA or linearized dsDNA, contains, from 5′ to 3′, homology region 1, a 2A-coding sequence fused directly to the CAR coding sequence followed by a polyA site, a 5′ LoxP site, a 5′ genomic insulator sequence, a splice acceptor to splice to AAVS1 exon 1, a 2A coding sequence, a coding sequence for a puromycin-resistance gene with a 2A peptide-linked coding sequence for the suicide gene HSV-TK, both driven off their own promoter and followed by a polyA signal sequence, the doxycycline-inducible Treg induction factor expression cassette, the rtTA coding sequence driven off a CAGG promoter, the coding sequence for a 4-OHT-inducible form of the Cre recombinase linked to the rtTA sequence via a 2A peptide and followed by a polyA sequence, a 3′ genomic insulator sequence, a 3′ LoxP site, and homology region 2. The genomic insulator sequences ensure the transgenes within them are not epigenetically silenced over the course of differentiation. The homology regions are homologous to the genomic regions flanking the ZFN cleavage site. Cells with successful targeted integration can be positively selected for by introducing puromycin into tissue culture (optionally waiting a week or more for unintegrated donor episomes to dilute out). The constitutively expressed 4-OHT-inducible Cre allows for excision of the entire cassette between the LoxP sites after addition of 4-OHT to culture. Cells which have not undergone recombinase-mediated excision will still express HSV-TK and thus can be eliminated by adding GCV into the tissue culture. This system allows for completely scarless removal of the Treg induction cassette while leaving the CAR cassette driven off the endogenous TRAC promoter integrated to allow for targeted immunosuppression in the engineered Tregs.

The above-described figures are merely illustrative of some embodiments of the present invention. For example, other self-cleaving peptides may be used in lieu of the T2A and P2A peptides illustrated in the figures. Self-cleaving peptides are viral derived peptides with a typical length of 18-22 amino acids. Self-cleaving 2A peptides include T2A, P2A, E2A, and F2A. Moreover, codon diversified versions of the 2A peptides may be used to combine multiple Treg induction genes on one large integrated transgene cassette. In some embodiments, IRES is used in in lieu of a self-cleaving peptide coding sequence. Both introns and exons may be targeted. Additional elements may be included in the heterologous sequence. For example, the heterologous sequence may include RNA-stabilizing elements such as a Woodchuck Hepatitis Virus Posttranscriptional Regulatory Element (WPRE).

3. Gene Editing Methods

Any gene editing method for targeted integration of a heterologous sequence into a specific genomic site may be used. To enhance the precision of site-specific integration of the transgene, a construct carrying the heterologous sequence may contain on either or both of its ends a homology region that is homologous to the targeted genomic site. In some embodiments, the heterologous sequence carries in both of 5′ and 3′ end regions sequences that are homologous to the target genomic site in a T cell specific gene locus or a genomic safe harbor gene locus. The lengths of the homology regions on the heterologous sequence may be, for example, 50-1,000 base pairs in length. The homology region in the heterologous sequence can be, but need not be, identical to the targeted genomic sequence. For example, the homology region in the heterologous sequence may be at 80 or more percent (e.g., 85 or more, 90 or more, 95 or more, 99 or more percent) homologous or identical to the targeted genomic sequence (e.g., the sequence that is to be replaced by the homology region in the heterologous sequence). In further embodiments, the construct, when linearized, comprise on one end homology region 1, and on its other end homology region 2, where homology regions 1 and 2 are respectively homologous to genomic region 1 and genomic region 2 flanking the integration site in the genome.

The construct carrying the heterologous sequence can be introduced to the target cell by any known techniques such as chemical methods (e.g., calcium phosphate transfection and lipofection), non-chemical methods (e.g., electroporation and cell squeezing), particle-based methods (e.g., magnetofection), and viral transduction (e.g., by using viral vectors such as vaccinia vectors, adenoviral vectors, lentiviral vectors, adeno-associated viral (AAV) vectors, retroviral vectors, and hybrid viral vectors). In some embodiments, the construct is an AAV viral vector and is introduced to the target human cell by a recombinant AAV virion whose genome comprises the construct, including having the AAV Inverted Terminal Repeat (ITR) sequences on both ends to allow the production of the AAV virion in a production system such as an insect cell/baculovirus production system or a mammalian cell production system. The AAV may be of any serotype, for example, AAV1, AAV2, AAV3, AAV4, AAVS, AAV6, AAV7, AAV8, AAV8.2, AAV9, or AAVrh10, of a pseudotype such as AAV2/8, AAV2/5, or AAV2/6.

The heterologous sequence may be integrated to the TRAC genomic locus by any site-specific gene knock-in technique. Such techniques include, without limitation, homologous recombination, gene editing techniques based on zinc finger nucleases or nickases (collectively “ZFNs” herein), transcription activator-like effector nucleases or nickases (collectively “TALENs” herein), clustered regularly interspaced short palindromic repeat systems (CRISPR, such as those using Cas9 or cpf1), meganucleases, integrases, recombinases, and transposes. As illustrated below in the Working Examples, for site-specific gene editing, the editing nuclease typically generates a DNA break (e.g., a single- or double-stranded DNA break) in the targeted genomic sequence such that a donor polynucleotide having homology to the targeted genomic sequence (e.g., the construct described herein) is used as a template for repair of the DNA break, resulting in the introduction of the donor polynucleotide to the genomic site.

Gene editing techniques are well known in the art. See, e.g., U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, 9,790,490, 10,000,772, 10,113,167, and 10,113,167 for CRISPR gene editing techniques. See, e.g., U.S. Pat. Nos. 8,735,153, 8,771,985, 8,772,008, 8,772,453, 8,921,112, 8,936,936, 8,945,868, 8,956,828, 9,234,187, 9,234,188, 9,238,803, 9,394,545, 9,428,756, 9,567,609, 9,597,357, 9,616,090, 9,717,759, 9,757,420, 9,765,360, 9,834,787, 9,957,526, 10,072,062, 10,081,661, 10,117,899, 10,155,011, and 10,260,062 for ZFN techniques and its applications in editing T cells and stem cells. The disclosures of the aforementioned patents are incorporated by reference herein in their entirety.

In gene editing techniques, the gene editing complex can be tailored to target specific genomic sites by altering the complex's DNA binding specificity. For example, in CRISPR technology, the guide RNA sequence can be designed to bind a specific genomic region; and in the ZFN technology, the zinc finger protein domain of the ZFN can be designed to have zinc fingers specific for a specific genomic region, such that the nuclease or nickase domains of the ZFN can cleave the genomic DNA at a site-specific manner. Depending on the desired genomic target site, the gene editing complex can be designed accordingly.

Components of the gene editing complexes may be delivered into the target cells, concurrent with or sequential to the transgene construct, by well-known methods such as electroporation, lipofection, microinjection, biolistics, virosomes, liposomes, lipid nanoparticles, immunoliposomes, polycation or lipid:nucleic acid conjugates, naked DNA or mRNA, and artificial virions. Sonoporation using, e.g., the Sonitron 2000 system (Rich-Mar) can also be used for delivery of nucleic acids. In particular embodiments, one or more components of the gene editing complex, including the nuclease or nickase, are delivered as mRNA into the cells to be edited.

4. Antigen-Specificity of the Tregs

In some embodiments, the stem cells or progenitor cells may be further engineered (e.g., using gene editing methods described herein) to include transgenes encoding an antigen-recognition receptor such as a TCR or a CAR. Alternatively, the stem cells or progenitor cells are cells that have been reprogrammed from mature Tregs that have already rearranged their TCR alpha/beta (or delta/gamma) loci, and Tregs re-differentiated from such stem or progenitor cells will retain the antigen specificity of their ancestral Tregs. In any event, the Tregs may be selected for their specificity for an antigen of interest for a particular therapeutic goal.

In some embodiments, the antigen of interest is a polymorphic allogeneic MHC molecule, such as one expressed by cells in a solid organ transplant or by cells in a cell-based therapy (e.g., bone marrow transplant, cancer CAR T therapy, or cell-based regenerative therapy). MHC molecules so targeted include, without limitation, HLA-A, HLA-B, or HLA-C; HLA-DP, HLA-DM, HLA-DOA, HLA-DOB, HLA-DQ, or HLA-DR. By way of example, the antigen of interest is class I molecule HLA-A2. HLA-A2 is a commonly mismatched histocompatibility antigen in transplantation. HLA-A mismatching is associated with poor outcomes after transplantation. Engineered Tregs expressing a CAR specific for an MHC class I molecule are advantageous because MHC class I molecules are broadly expressed on all tissues, so the Tregs can be used for organ transplantation regardless of the tissue type of the transplant. Tregs against HLA-A2 offers the additional advantage that HLA-A2 is expressed by a substantial proportion of the human population and therefore on many donor organs. There has been evidence showing that expression of an HLA-A2 CAR in Treg cells can enhance the potency of the Treg cells in preventing transplant rejection (see, e.g., Boardman, supra; MacDonald et al., J Clin Invest. (2016) 126(4):1413-24; and Dawson, supra).

In some embodiments, the antigen of interest is an autoantigen, i.e., an endogenous antigen expressed prevalently or uniquely at the site of autoimmune inflammation in a specific tissue of the body. Tregs specific for such an antigen can home to the inflamed tissue and exert tissue-specific activity by causing local immunosuppression. Examples of autoantigens are aquaporin water channels (e.g., aquaporin-4 water channel), paraneoplastic antigen Ma2, amphiphysin, voltage-gated potassium channel, N-methyl-d-aspartate receptor (NMDAR), a-amino-3-hydroxy-5-methyl-4-isoxazoleproprionic acid receptor (AMPAR), thyroid peroxidase, thyroglobulin, anti-N-methyl-D-aspartate receptor (NR1 subunit), Rh blood group antigens, desmoglein 1 or 3 (Dsg1/3), BP180, BP230, acetylcholine nicotinic postsynaptic receptors, thyrotropin receptors, platelet integrin, glycoprotein IIb/IIIa, calpastatin, citrullinated proteins, alpha-beta-crystallin, intrinsic factor of gastric parietal cells, phospholipase A2 receptor 1 (PLA2R1), and thrombospondin type 1 domain-containing 7A (THSD7A). Additional examples of autoantigens are multiple sclerosis-associated antigens (e.g., myelin basic protein (MBP), myelin associated glycoprotein (MAG), myelin oligodendrocyte glycoprotein (MOG), proteolipid protein (PLP), oligodendrocyte myelin oligoprotein (OMGP), myelin associated oligodendrocyte basic protein (MOBP), oligodendrocyte specific protein (OSP/Claudin 11), oligodendrocyte specific proteins (OSP), myelin-associated neurite outgrowth inhibitor NOGO A, glycoprotein Po, peripheral myelin protein 22 (PMP22), 2′3′-cyclic nucleotide 3′-phosphodiesterase (CNPase), and fragments thereof); joint-associated antigens (e.g., citrulline-substituted cyclic and linear filaggrin peptides, type II collagen peptides, human cartilage glycoprotein 39 peptides, keratin, vimentin, fibrinogen, and type I, III, IV, and V collagen peptides); and eye-associated antigens (e.g., retinal arrestin. S-arrestin, interphotoreceptor retinoid-binding proteins, beta-crystallin B1, retinal proteins, choroid proteins, and fragments thereof). In some embodiments, the autoantigen targeted by the Treg cells is IL23-R (for treatment of, e.g., Crohn's disease, inflammatory bowel disease, or rheumatoid arthritis), MOG (for treatment of multiple sclerosis), or MBP (for treatment of multiple sclerosis). In some embodiments, the Tregs may target other antigens of interest (e.g., B cell markers CD19 and CD20).

In some embodiments, Tregs recognizing foreign peptides (e.g., CMV, EBV, and HSV), rather than allo-antigens, can be used in an allogeneic adoptive cell transfer setting without the risk of being constantly activated by recognizing allo-antigens without the need for knockout out of TCR expression.

Additional Genome Editing

The present engineered cells may be further genetically engineered, before or after the genome editing described above, to make the cells more effective, more useable on a larger patient population, and/or safer. The genetic engineering may be done by, e.g., random insertion of a heterologous sequence of interest (e.g., by using a lentiviral vector, a retroviral vector, or a transposon) or targeted genomic integration (e.g., by using genome editing mediated by ZFN, TALEN, CRISPR, site-specific engineered recombinase, or meganuclease).

For example, the cells may be engineered to express one or more exogenous CAR or TCR through a site-specific integration of a CAR or TCR transgene into the genome of the cell. The exogenous CAR or TCR may target an antigen of interest, as described above.

The cells may also be edited to encode one or more therapeutic agents to promote the immunosuppressive activity of the Tregs. Examples of therapeutic agents include cytokines (e.g., IL-10), chemokines (e.g., CCR7), growth factors (e.g., remyelination factors for treatment of multiple sclerosis), and signaling factors (e.g., amphiregulin).

In additional embodiments, the cells are further engineered to express a factor that reduces severe side effects and/or toxicities of cell therapy, such as cytokine release syndrome (CRS) and/or neurotoxicities (e.g., an anti-IL-6 scFv or a secretable IL-12) (see, e.g., Chmielewski et al., Immunol Rev. (2014) 257(1):83-90).

In some embodiments, EZH1 signaling is disrupted in the engineered cells to enhance their lymphoid commitment (see, e.g., Vo et al., Nature (2018) 553(7689):506-10).

In some embodiments, the edited cells may be allogeneic cells to the patient. In such instances, the cells may be further engineered to reduce host rejection to these cells (graft rejection) and/or these cells' potential attack on the host (graft-versus-host disease). The further-engineered allogeneic cells are particularly useful because they can be used in multiple patients without compatibility issues. The allogeneic cells thus can be called “universal” and can be used “off the shelf” The use of “universal” cells greatly improves the efficiency and reduces the costs of adopted cell therapy.

To generate “universal” allogeneic cells, the cells may be engineered, for example, to have a null genotype for one or more of the following: (i) T cell receptor (TCR alpha chain or beta chain); (ii) a polymorphic major histocompatibility complex (MHC) class I or II molecule (e.g., HLA-A, HLA-B, or HLA-C; HLA-DP, HLA-DM, HLA-DOA, HLA-DOB, HLA-DQ, or HLA-DR; or (32-microglobulin (B2M)); (iii) a transporter associated with antigen processing (e.g., TAP-1 or TAP-2); (iv) Class II MHC transactivator (CIITA); (v) a minor histocompatibility antigen (MiHA; e.g., HA-1/A2, HA-2, HA-3, HA-8, HB-1H, or HB-1Y); (vi) immune checkpoint inhibitors such as PD-1 and CTLA-4; (vii) VIM; and (vi) any combination thereof.

The allogeneic engineered cells may also express an invariant HLA or CD47 to increase the resistance of the engineered cells (especially those with HLA class I knockout or knockdown) to the host's natural killer and other immune cells involved in anti-graft rejection. For example, the heterologous sequence carrying the commitment factor transgene may additionally comprise a coding sequence for an invariant HLA (e.g., HLA-G, HLA-E, and HLA-F) or CD47. The invariant HLA or CD47 coding sequence may be linked to the primary transgene in the heterologous sequence through a coding sequence for a self-cleaving peptide or an IRES sequence.

6. Safety Switch in Engineered Cells

In cell therapy, it may be desirable for the transplanted cells to contain a “safety switch” in their genomes, such that proliferation of the cells can be stopped when their presence in the patient is no longer desired (see, e.g., Hartmann et al., EMBO Mol Med. (2017) 9:1183-97). A safety switch may, for example, be a suicide gene, which upon administration of a pharmaceutical compound to the patient, will be activated or inactivated such that the cells enter apoptosis. A suicide gene may encode an enzyme not found in humans (e.g., a bacterial or viral enzyme) that converts a harmless substance into a toxic metabolite in the human cell.

In some embodiments, the suicide gene may be a thymidine kinase (TK) gene from Herpes Simplex Virus (HSV). TK can metabolize ganciclovir, valganciclovir, famciclovir, or another similar antiviral drug into a toxic compound that interferes with DNA replication and results in cell apoptosis. Thus, an HSV-TK gene in a host cell can be turned on to kill the cell by administration of one of such antiviral drugs to the patient.

In other embodiments, the suicide gene encodes, for example, another thymidine kinase, a cytosine deaminase (or uracil phosphoribosyltransferase; which transforms anti-fungal drug 5-fluorocytosine into 5-fluorouracil), a nitroreductase (which transforms CB1954 (for [5-(aziridin-1-yl)-2,4-dinitrobenzamide]) into a toxic compound), 4-hydroxylamine), and a cytochrome P450 (which transforms ifosfamide to acrolein (nitrogen mustard)) (Rouanet et al., Int J Mol Sci. (2017) 18(6):E1231), or inducible caspase-9 (Jones et al., Front Pharmacol. (2014) 5:254). In additional embodiments, the suicide gene may encode an intracellular antibody, a telomerase, another caspase, or a DNAase. See, e.g., Zarogoulidis et al., J Genet Syndr Gene Ther. (2013) doi:10.4172/2157-7412.1000139.

A safety switch may also be an “on” or “accelerator” switch, a gene encoding a small interfering RNA, an shRNA, or an antisense that interferences the expression of a cellular protein critical for cell survival.

The safety switch may utilize any suitable mammalian and other necessary transcription regulatory sequences. The safety switch can be introduced into the cell through random integration or site-specific integration using gene editing techniques described herein or other techniques known in the art. It may be desirable to integrate the safety switch in a genomic safe harbor such that the genetic stability and the clinical safety of the engineered cell are maintained. Examples of safe harbors as used in the present disclosure are the AAVS1 locus; the ROSA26 locus; the CLYBL locus; the gene loci for albumin, CCR5, and CXCR4; and the locus where the endogenous gene is knocked out in the engineered cells (e.g., the T cell receptor alpha or beta chain gene locus, the HLA gene locus, the CIITA locus, or the β2-microglobulin gene locus).

### III. Use of the Teff and Treg Cells

The Teff and Treg cells of the present disclosure can be used in cell therapy to treat a patient (e.g., a human patient) in need of induction of immune tolerance or restoration of immune homeostasis. The terms “treating” and “treatment” refer to alleviation or elimination of one or more symptoms of the treated condition, prevention of the occurrence or reoccurrence of the symptoms, reversal or remediation of tissue damage, and/or slowing of disease progression.

A patient herein may be one having or at risk of having an undesired inflammatory condition such as an autoimmune disease. Examples of autoimmune diseases are Addison's disease, AIDS, ankylosing spondylitis, anti-glomerular basement membrane disease autoimmune hepatitis, dermatitis, Goodpasture's syndrome, granulomatosis with polyangiitis, Graves' disease, Guillain-Barre syndrome, Hashimoto's thyroiditis, hemolytic anemia, Henoch-Schonlein purpura (HSP), juvenile arthritis, juvenile myositis, Kawasaki disease, inflammatory bowel diseases (such as Crohn's disease and ulcerative colitis), polymyositis, pulmonary alveolar proteinosis, multiple sclerosis, myasthenia gravis, neuromyelitis optica, PANDAS, psoriasis, psoriatic arthritis, rheumatoid arthritis, Sjögren's syndrome, systemic scleroderma, systemic sclerosis, systemic lupus erythematosus, thrombocytopenic purpura (TTP), Type I diabetes mellitus, uveitis, vasculitis, vitiligo, and Vogt-Koyanagi-Harada Disease.

In some embodiments, the Tregs express an antigen-binding receptor (e.g., TCR or CAR) targeting an autoantigen associated with an autoimmune disease, such as myelin oligodendrocyte glycoprotein (multiple sclerosis), myelin protein zero (autoimmune peripheral neuropathy), HIV env or gag protein (AIDS), myelin basic protein (multiple sclerosis), CD37 (systemic lupus erythematosus), CD20 (B-cell mediated autoimmune diseases), and IL-23R (inflammatory bowel diseases such as Crohn's disease or ulcerative colitis).

A patient herein may be one in need of an allogeneic transplant, such as an allogeneic tissue or solid organ transplant or an allogeneic cell therapy. The Tregs of the present disclosure, such as those expressing CARs targeting one or more allogeneic MHC class I or II molecules, may be introduced to the patient, where the Tregs will home to the transplant and suppress allograft rejection elicited by the host immune system and/or graft-versus-host rejection. Patients in need of a tissue or organ transplant or an allogeneic cell therapy include those in need of, for example, kidney transplant, heart transplant, liver transplant, pancreas transplant, intestine transplant, vein transplant, bone marrow transplant, and skin graft; those in need of regenerative cell therapy; those in need of gene therapy (AAV-based gene therapy); and those in need in need of cancer CAR T therapy.

If desired, the patient receiving the engineered Tregs herein (which includes patients receiving engineered pluripotent or multipotent cells that will differentiate into Tregs in vivo) is treated with a mild lymphodepletion procedure prior to introduction of the cell graft or with a vigorous myeloablative conditioning regimen.

In some embodiments, the present Teffs are engineered to express an antigen receptor such as a modified or unmodified TCR, or a chimeric antigen receptor. The antigen receptor targets an antigen of interest. The Teffs may increase inflammatory responses and promote the activity of other immune cells (e.g., NK cells or CTLs) for killing harmful cells. Harmful cells may be, for example: tumor cells in oncology settings; cells infected by viruses or other pathogens in infectious disease settings; autoantibody-producing B cells or self-reactive cells in autoimmune or inflammatory settings; and pathogenic allergen-responsive cells in allergy settings. In some embodiments, the Teff cells can be used cell replacement therapies in patients with defective CD4⁺ compartments (e.g., AIDS).

The engineered cells of the present disclosure may be provided in a pharmaceutical composition containing the cells and a pharmaceutically acceptable carrier. For example, the pharmaceutical composition comprises sterilized water, physiological saline or neutral buffered saline (e.g., phosphate-buffered saline), salts, antibiotics, isotonic agents, and other excipients (e.g., glucose, mannose, sucrose, dextrans, mannitol; proteins (e.g., human serum albumin); amino acids (e.g., glycine and arginine); antioxidants (e.g., glutathione); chelating agents (e.g., EDTA); and preservatives). The pharmaceutical composition may additionally comprise factors that are supportive of the Treg phenotype and growth (e.g., IL-2 and rapamycin or derivatives thereof), anti-inflammatory cytokines (e.g., IL-10, TGF-β, and IL-35), and other cells for cell therapy (e.g., CART effector cells for cancer therapy or cells for regenerative therapy). For storage and transportation, the cells optionally may be cryopreserved. Prior to use, the cells may be thawed and diluted in a pharmaceutically acceptable carrier.

The pharmaceutical composition of the present disclosure is administered to a patient in a therapeutically effective amount through systemic administration (e.g., through intravenous injection or infusion) or local injection or infusion to the tissue of interest (e.g., infusion through the hepatic artery, and injection to the brain, heart, or muscle). The term “therapeutically effective amount” refers to the amount of a pharmaceutical composition, or the number of cells, that when administered to the patient, is sufficient to effect the treatment.

In some embodiments, a single dosing unit of the pharmaceutical composition comprises more than 10⁴ cells (e.g., from about 10⁵ to about 10⁶ cells, from about 10⁶ to about 10¹⁰, from about 10⁶ to 10⁷, from about 10⁶ to 10⁸, from about 10⁷ to 10⁸, from about 10⁷ to 10⁹, or from about 10⁸ to 10⁹ cells). In certain embodiments, a single dosing unit of the composition comprises about 10⁶, about 10⁷, about 10⁸, about 10⁹, or about 10¹⁰ or more cells. The patient may be administered with the pharmaceutical composition once every two days, once every three days, once every four days, once a week, once every two weeks, once every three weeks, once a month, or at another frequency as necessary to establish a sufficient population of engineered Treg cells in the patient.

Pharmaceutical compositions comprising any of the zinc finger nucleases or other nucleases and polynucleotides as described herein are also provided.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, immunology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

### Example 1: Feeder-Free Derivation of TiPSC Lines

This example describes a process in which iPSC cell lines were derived from T cells (TiPSC lines) by using reprogramming factors. In the process, iPSCs were derived from a mixed population of cryopreserved CD4⁺ and CD8⁺ T cells previously isolated from a leukapheresis pack (AllCells, USA) from a healthy human donor. The cells were thawed and cultured in RPMI+10% human AB serum (huABS) supplemented with 100 U/mL IL-2. The cells were activated with CD3/CD28 Dynabeads at a ratio of 1:1 (cells:beads). The activated cells were plated at a density of 10⁶ cells/mL and cultured overnight at 37° C., 5% CO₂.

After overnight culture, cells were stained and sorted for total CD4⁺ T cells, total CD8⁺ T cells, and naïve Tregs (CD4⁺CD25^{high}CD127^{low}CD45RA⁺). Following the sort, CD4⁺ and CD8⁺ T cells recovered in RPMI+10% huABS+100 U/mL IL-2 and naïve Tregs recovered in RPMI+10% huABS+1000 U/mL IL-2 overnight.

Sorted T cells were reprogrammed the following day using Cytotune-iPS 2.0 Sendai Reprogramming Kit (Thermo Fisher, USA) following manufacturer's instructions. Approximately 0.5×10⁶ cells from each sorted population were transduced by spin infection with Sendai virus (SeV) carrying reprogramming factors (Oct3/4, Sox2, Klf4, and c-Myc) and plated into their respective culture medium without removal of the virus.

One day after infection, cells were harvested and plated onto Matrigel in ReproTeSR (StemCell Technologies, Canada). ReproTeSR was added to the wells every 1-2 days without media removal until day 7. After day 7, daily media changes were performed. Approximately 2-3 weeks after reprogramming, iPSC colonies were manually picked based on morphology. Colonies were manually passaged until morphology stabilized and were then expanded in mTeSR media (StemCell Technologies) for several more weeks.

### Example 2: Differentiation of iPSCs to Hematopoietic Stem and Progenitor Cells

This example describes a process in which iPSCs were differentiated into hematopoietic stem and progenitor cells (HSPCs) in tissue culture. The process is illustrated in FIG. 1.

In this process, the starting iPSCs were plated in APEL2 medium (StemCell Technologies) in the presence of 10 ng/mL of BMP4, 10 ng/mL VEGF, 50 ng/mL SCF, 10 ng/mL bFGF, and 10 μM ROCK inhibitor (Y-27632 dihydrochloride) on day 0. Cells were briefly centrifuged to enhance EB formation.

On day 1 or day 2 after EBs formed, a full media change was performed by replacing the media from all wells with APEL2 media containing 20 ng/mL VEGF, 10 ng/mL bFGF, 100 ng/mL SCF, 20 ng/mL Flt-3, 20 ng/mL TPO, and 40 ng/mL IL-3. Media was changed every 2 to 3 days until day 8.

On day 8, a full media change was performed by replacing the media with complete StemPro-34 medium (Thermo Fisher, USA) supplemented with non-essential amino acids, GlutaMAX™ and 0.1 mM beta-mercaptoethanol and containing 100 ng/mL SCF, 20 ng/mL Flt-3, 20 ng/mL TPO, and 40 ng/mL IL-3. Media was changed every 2 to 3 days for an additional 6 to 7 days.

On day 14 or day 15 of total differentiation, HSPCs were collected and strained through a cell strainer to separate EBs from extruded HSPCs.

### Example 3: Differentiation of iPSC-HSPCs to CD4sp T Cells and Tregs

This example described a process in which iPSC-derived HSPCs (iPSC-HSPCs) were differentiated into CD4sp T cells and Tregs. This process is illustrated in FIG. 1 and FIG. 2

In this process, iPSC-HSPCs were plated on tissue culture-treated plates pre-coated with Lymphoid Differentiation Coating Material in StemSpan™ Lymphoid Progenitor Expansion Medium (StemCell Technologies) at a density of 1×10⁴-5×10⁴ cells/mL (day 14). Cells were fed on day 17 or 18, day 21, and day 24 or 25 following manufacturer's instructions until day 28 to generate lymphoid progenitors (T cell precursor).

On day 28, lymphoid progenitors were harvested and plated on fresh plates coated with Lymphoid Differentiation Coating Material in StemSpan™ T Cell Progenitor Maturation Medium at a density of 0.2×10⁶ cells/mL. Cells were fed every 3 or 4 days until day 35 or day 42.

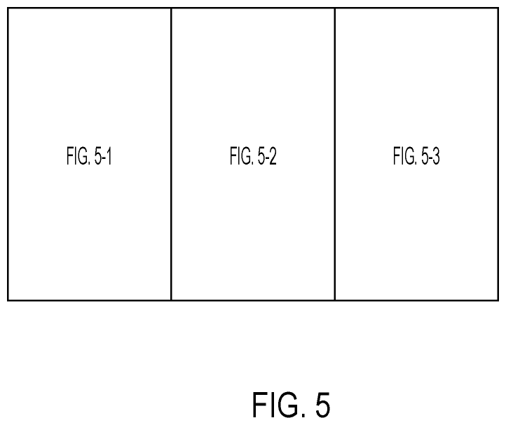

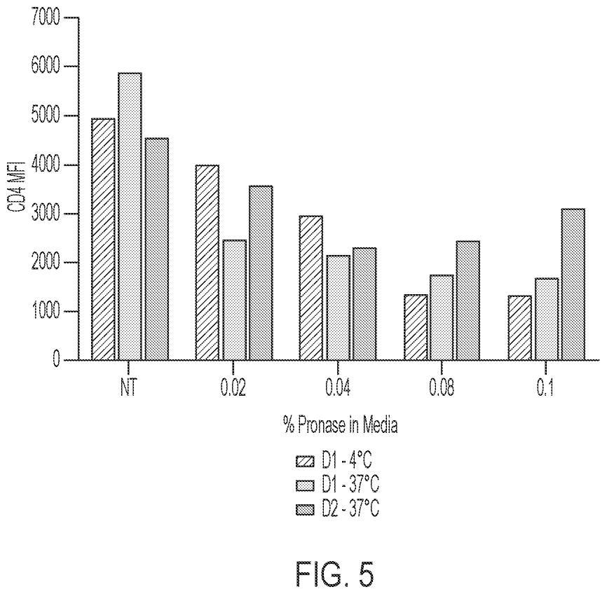

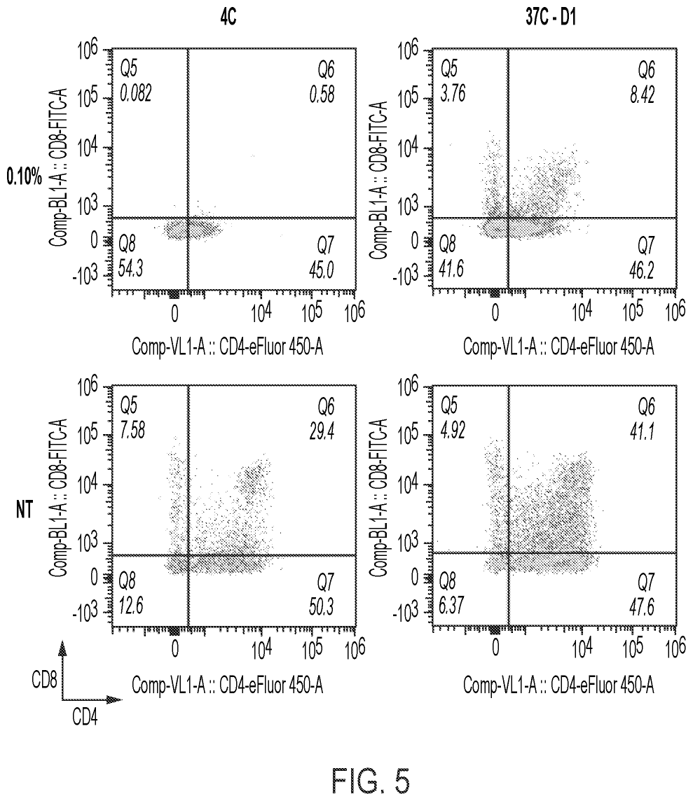

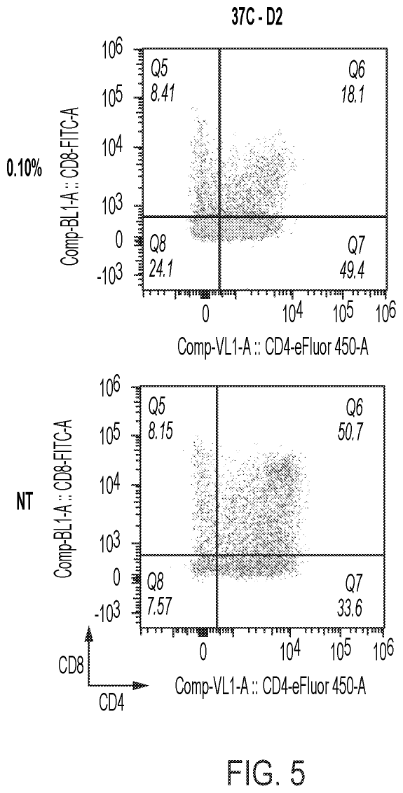

To generate CD4sp T cells, on either day 35 or day 42, cells were treated with 0.00625 to 0.1 ng/μL, phorbol 12-myristate 13-acetate (PMA) and 0.125 to 2 ng/μL, ionomycin (I) (0.125×-2×PMAI) to generate CD4sp T cells. PMAI was removed from the cultures through a full media change with StemSpan™ T Cell Progenitor Maturation Medium 24 hours after treatment. Treated cells were fed every 3-4 days for an additional 7 days. PMAI-treated iPSC-derived CD4sp T cells were also subjected to pronase treatment to determine commitment to the CD4 lineage. Cells were treated at an increasing percentage of pronase in the culture medium up to 0.1% and either incubated at 4° C. to prevent CD4 re-expression or at 37° C. to allow for re-expression. The mean fluorescence intensity (MFI) of CD4 and CD8 expression was examined one and two days after treatment by flow cytometry. The data show that CD4sp T cells were able to re-express CD4 and CD8 after pronase treatment, demonstrating that these cells prior to pronase treatment were committed to the lineage (FIG. 5).

To generate iPSC-derived Tregs, CD4sp T cells were treated with Immunocult™ Human Treg Differentiation Supplement (StemCell Technologies) containing human recombinant TGF-β1, all-trans retinoic acid (ATRA), and IL-2 on day 43 or day 50 according to manufacturer's instructions (1 mL of supplement per 49 mL of media). Cells were also activated with human CD3/CD28/CD2 T cell activators (StemCell Technologies) (12.5 μL/mL) on the day of TGF-β1 and ATRA treatment. Cells were fed every 3-4 days for the next 7 days.

Tregs derived from iPSC were also generated by treating CD4sp T cells with ImmunoCult™ Human Treg Differentiation Supplement (StemCell Technologies) (1 mL of supplement per 49 mL of media), activated with human CD3/CD28/CD2 T cell activators (StemCell Technologies) (12.5 μL/mL), and supplemented with IL-2 in a 50% mixture of StemSpan™ T Cell Progenitor Maturation Medium and a T cell-specific medium (OpTmizer™, ImmunoCult™, or X-VIVO™ 15). Cells were fed every 3-4 days for the next 7 days.

Seven days after the first TGF-β1 and ATRA treatment, cells were stimulated with 12.5-50 μL/mL of CD3/CD28/CD2 T cell activators supplemented with 10-1000 U/mL IL-2 and used in downstream functional or characterization assays.

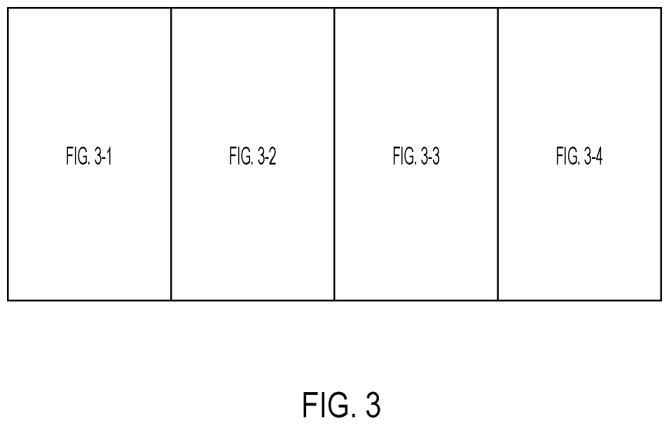

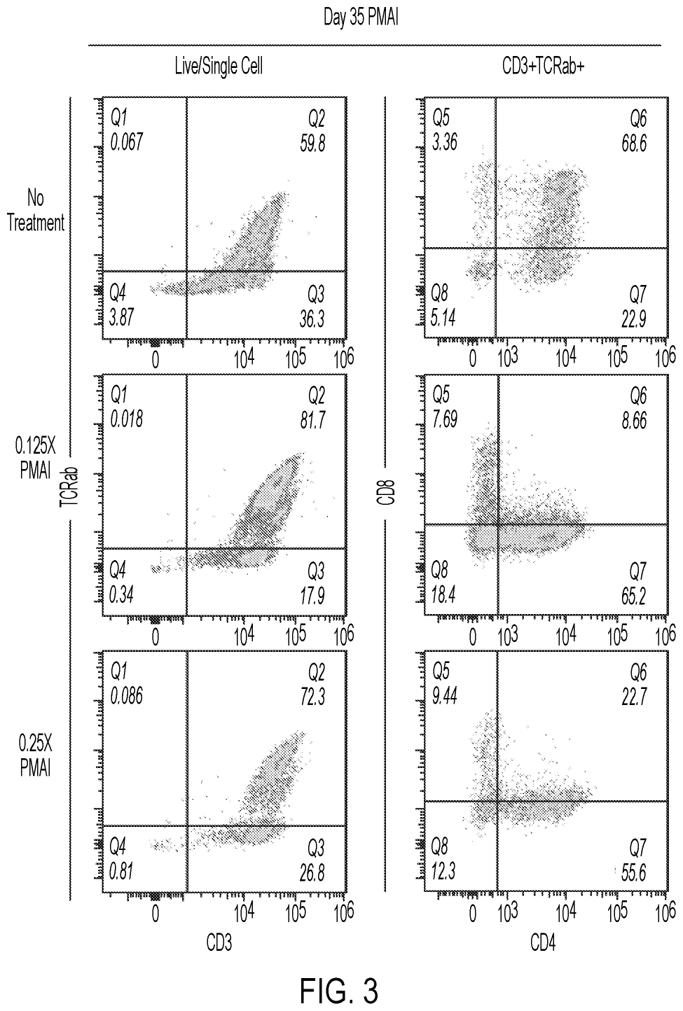

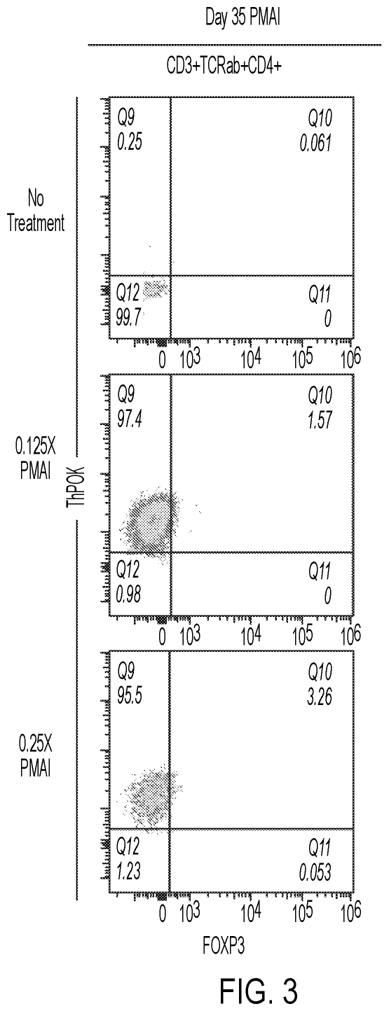

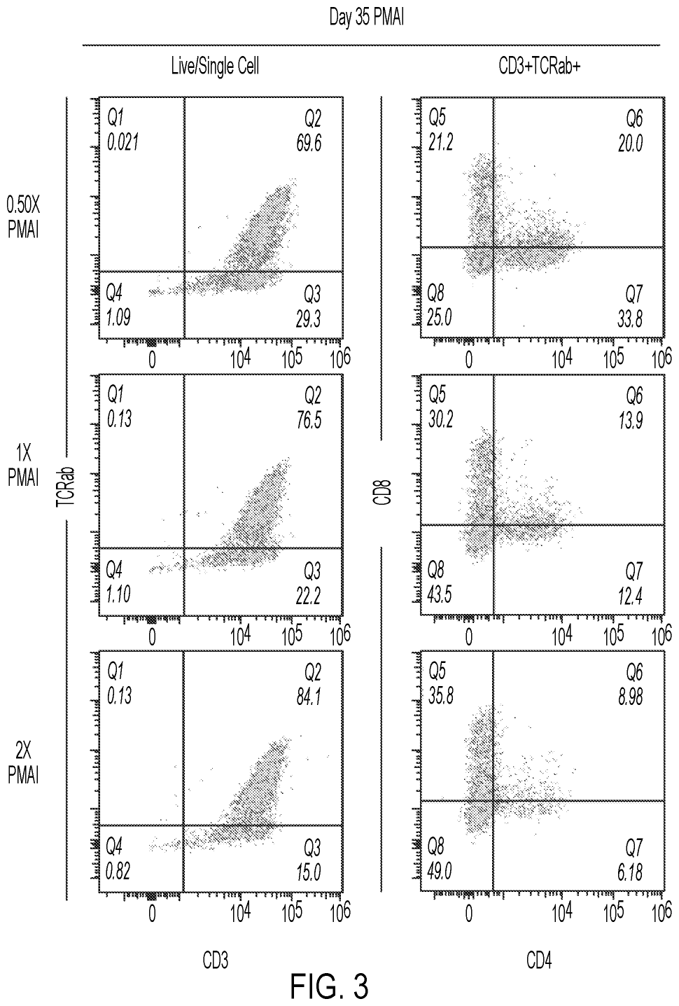

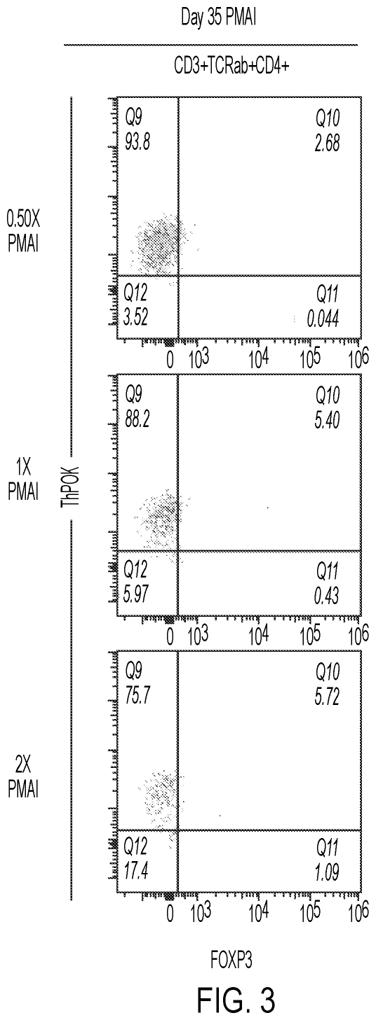

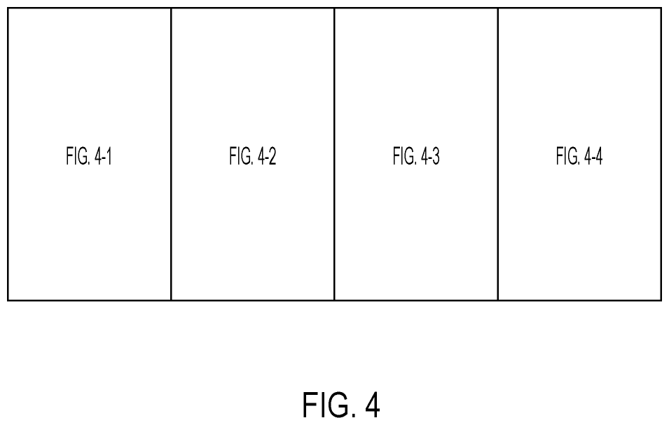

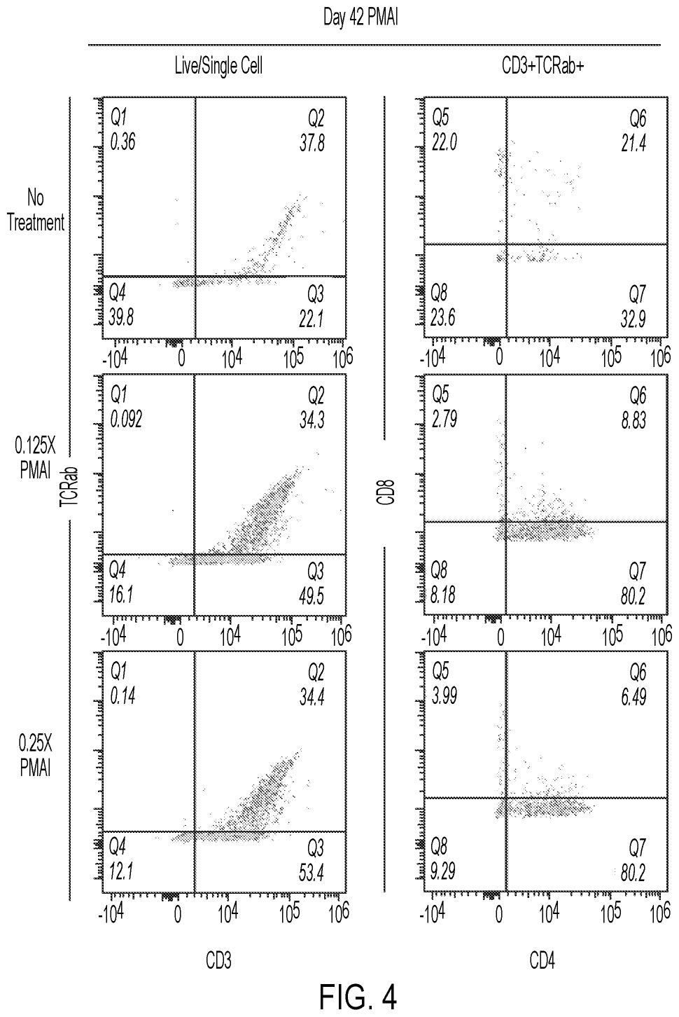

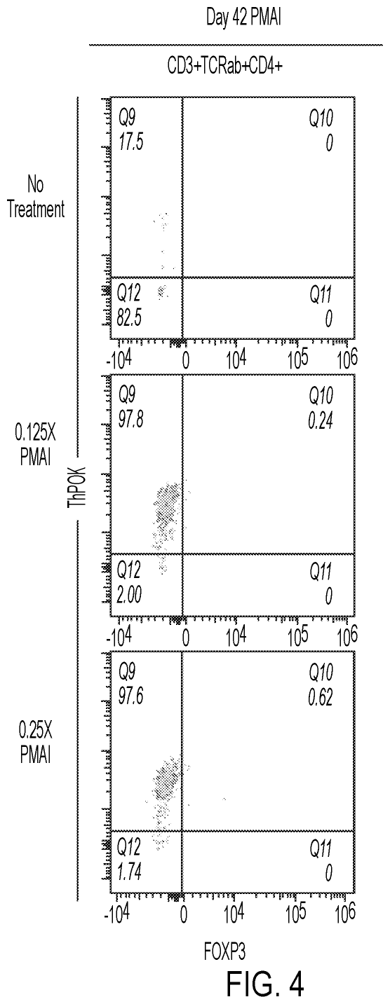

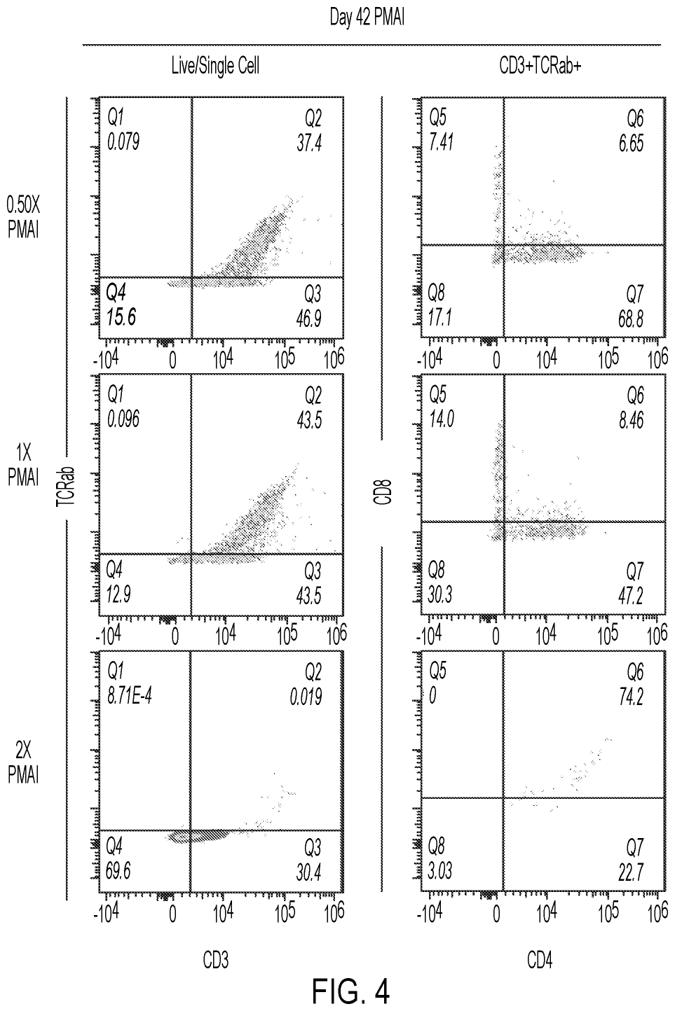

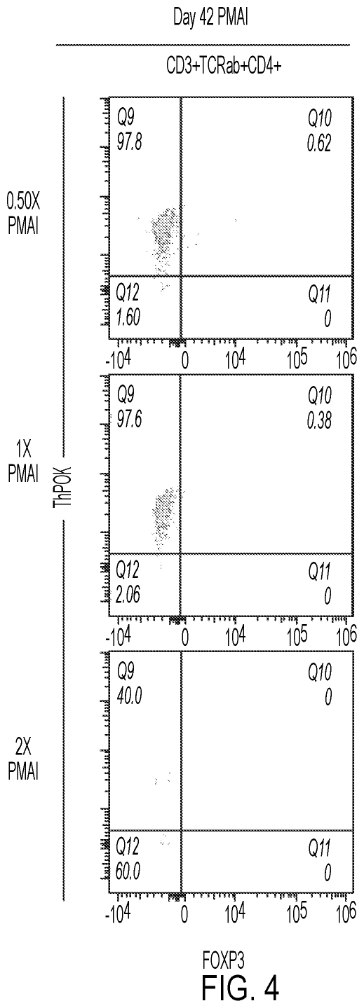

Flow cytometry analysis demonstrates the ability of PMAI to differentiate double positive T cells to CD4sp cells at two timepoints during their development: PMAI added at day 35 (FIG. 3) or day 42 (FIG. 4). PMAI was added at various concentrations with the lower concentrations (0.125× to 0.25×) producing higher percentages of CD4sp cells compared to the higher concentrations of PMAI. PMAI-treated CD4⁺CD8⁻ cells (Q7 in all plots) expressed ThPOK but not FOXP3 (Q9 in all plots). The cells also expressed CD3 and TCR αβ. Double positive cells that were not treated with PMAI remained mainly double positive and CD4⁺CD8⁻ cells did not express ThPOK.

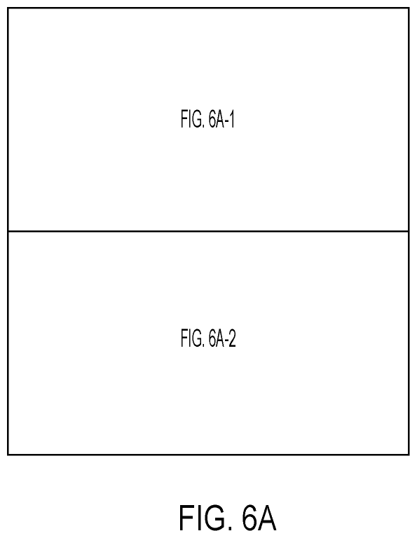

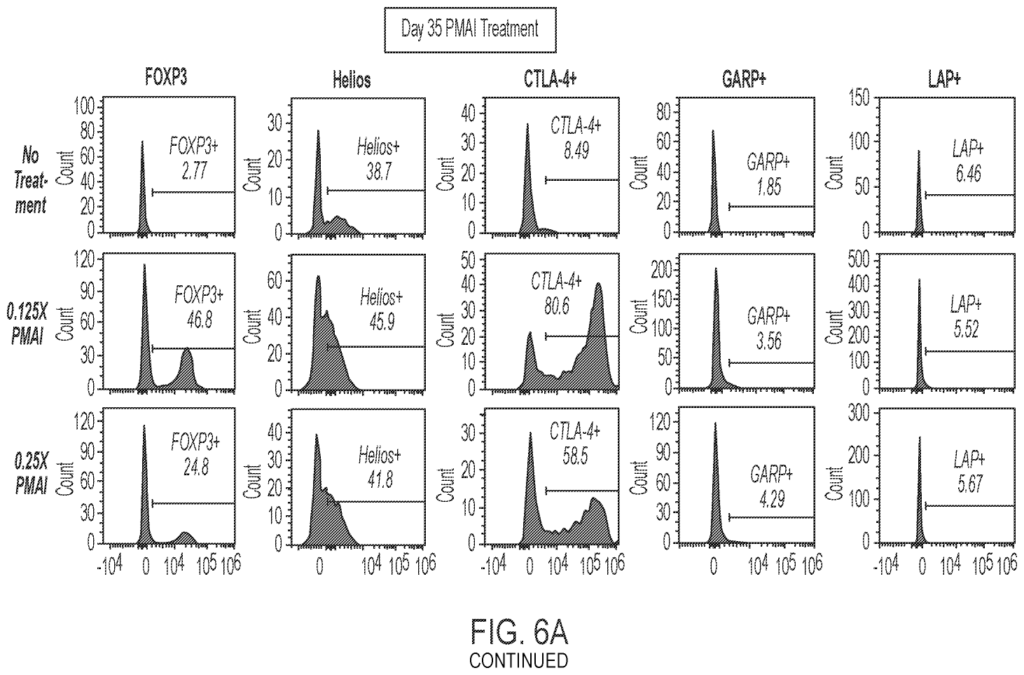

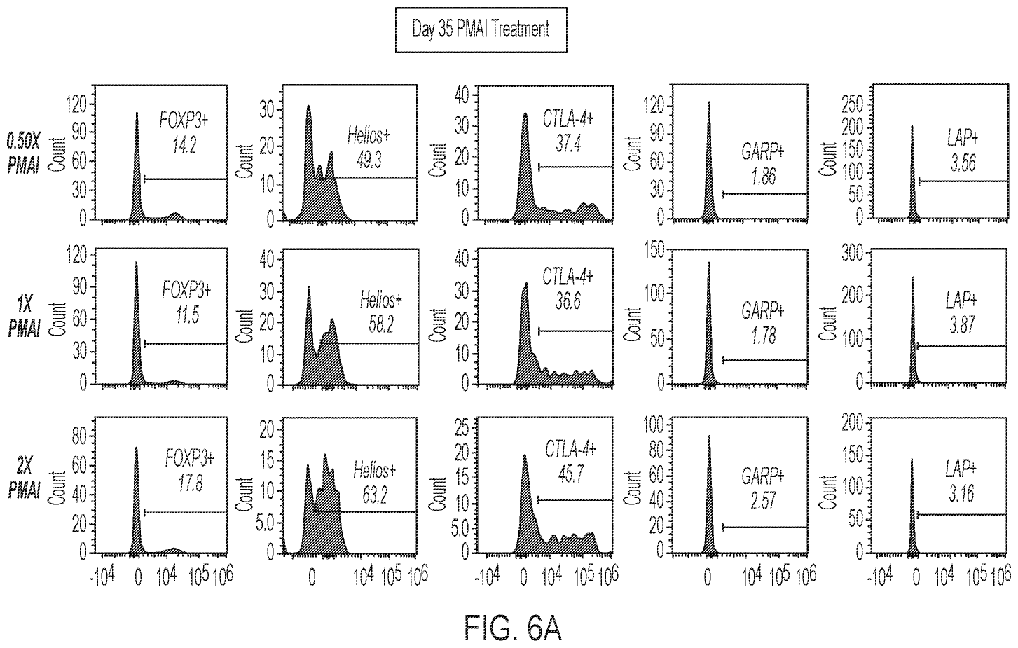

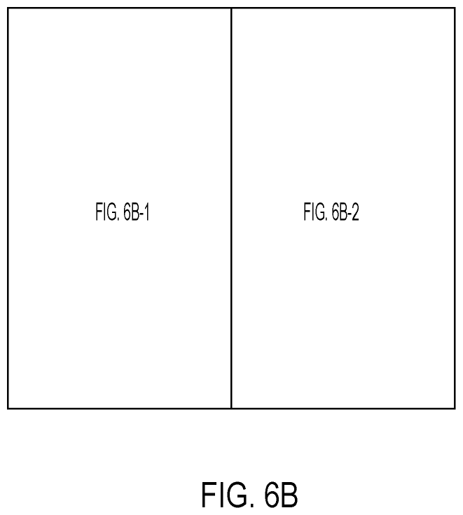

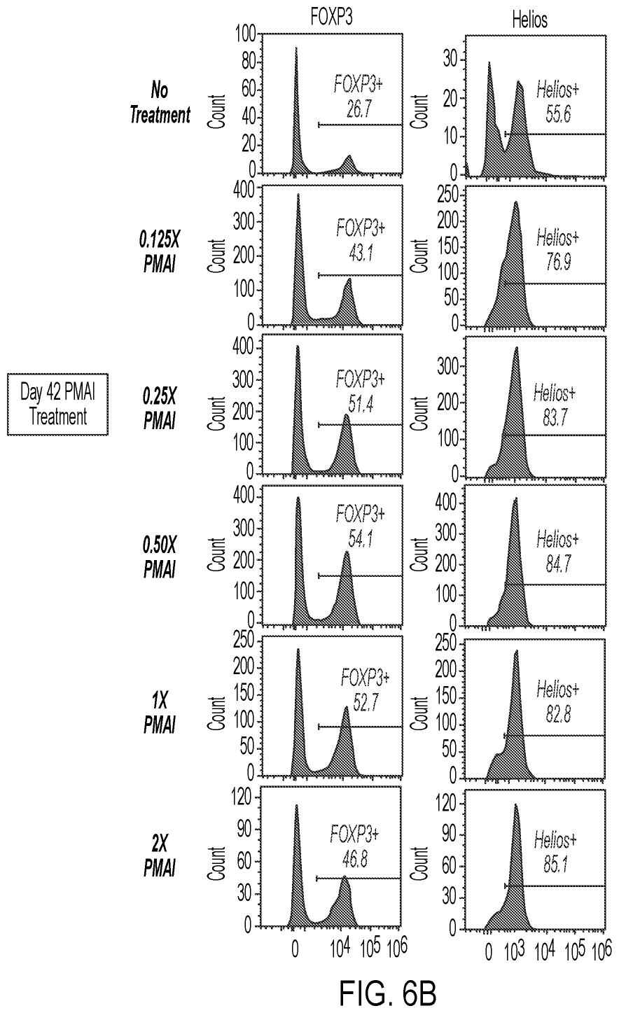

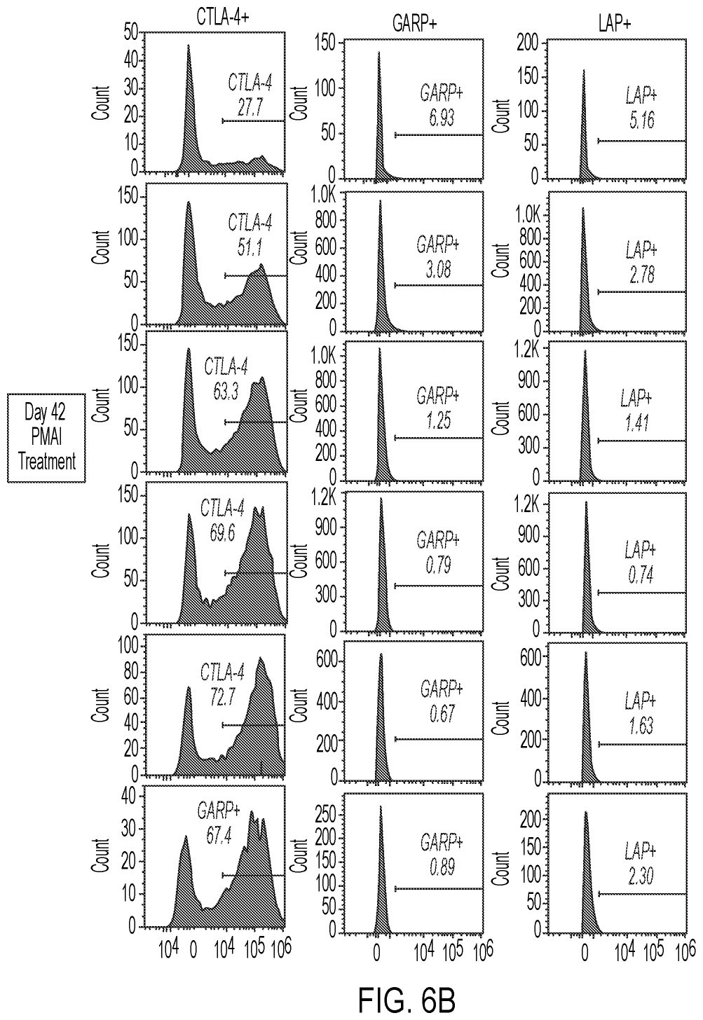

The identity and function of the CD4sp T cells were further analyzed. Flow cytometry analysis showed the expression of activated Treg markers in PMAI-induced CD4sp T cells (PMAI added at day 35 or day 42) that were treated with TGF-β1, ATRA, IL-2 and CD3/CD28/CD2 T cell activators. FOXP3 could be observed at the lower concentrations of PMAI (0.125× and 0.25×) added to double positive cells at day 35 of differentiation (FIG. 6A) but was highly expressed in all concentrations of PMAI added at day 42 of differentiation (FIG. 6B). The cells also expressed Helios and CTLA-4 but did not express LAP or GARP. Cells that were not treated with PMAI expressed little to no FOXP3, CTLA-4, GARP, and LAP in spite of TGF-β1 and ATRA treatment. Helios was also expressed on these cells.

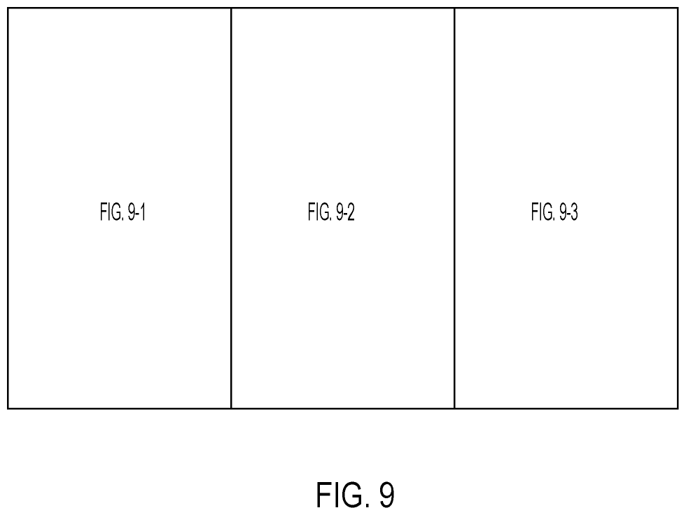

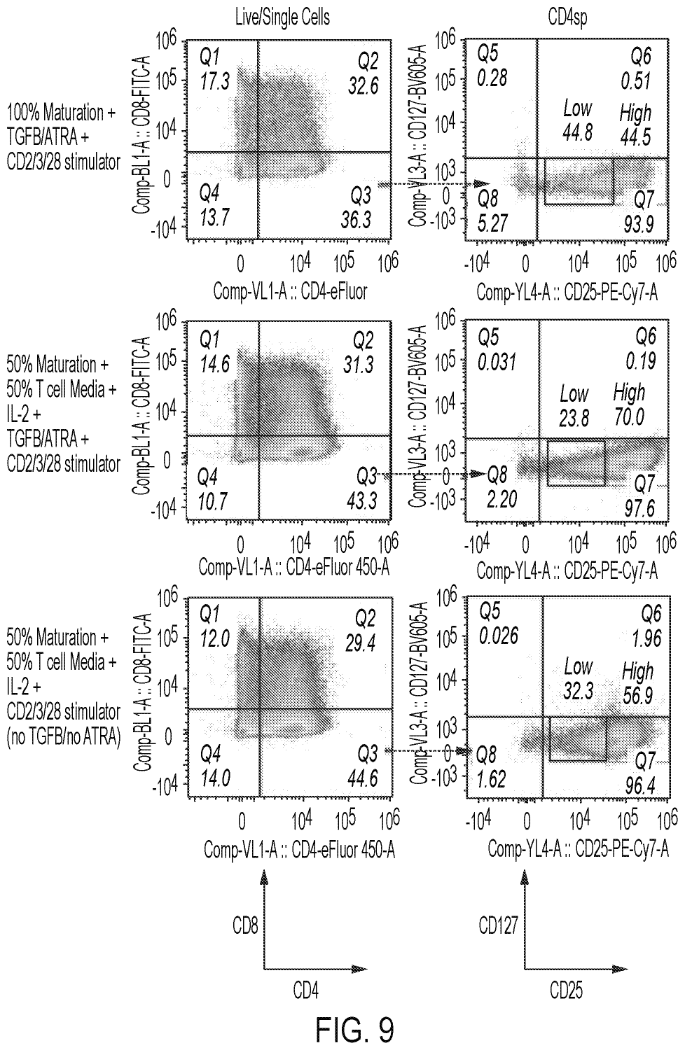

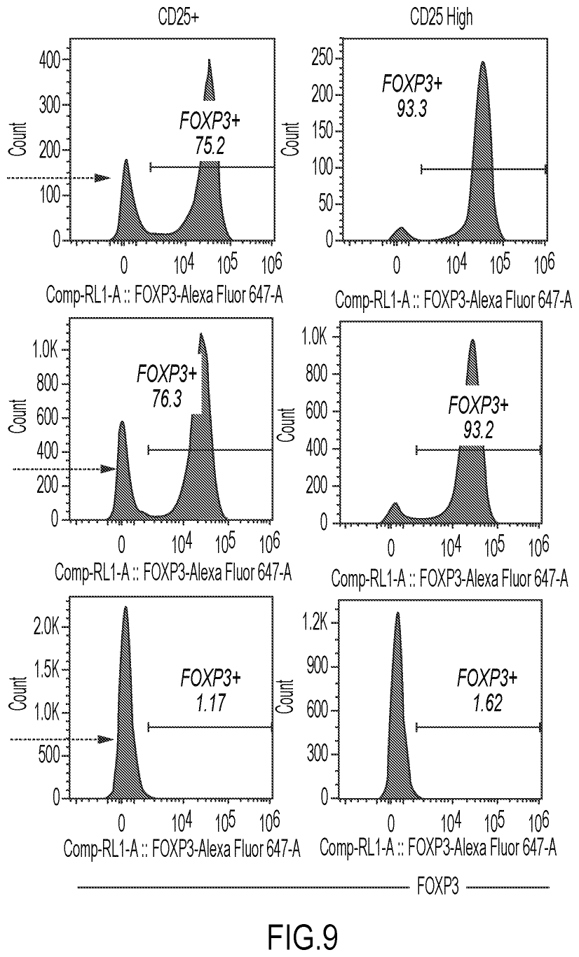

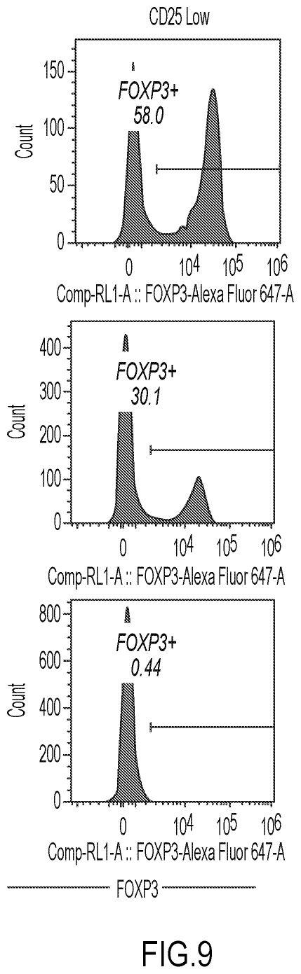

Further characterization of PMAI-induced and TGF-β1-treated iPSC-Tregs also showed the expression of CD25 and little-to-no expression of CD127 in the CD4sp population. Within the CD25⁺ population, the majority of the cells (75%) was FOXP3⁺. Further sub-gating within the CD25⁺ population shows that cells highly expressing CD25 (CD25^{high}) were greater than 90% FOXP3⁺, while cells expressing low/moderate levels of CD25 (CD25^{low}) contained a mixture of FOXP3⁺ and FOXP3⁻ cells (FIG. 9).

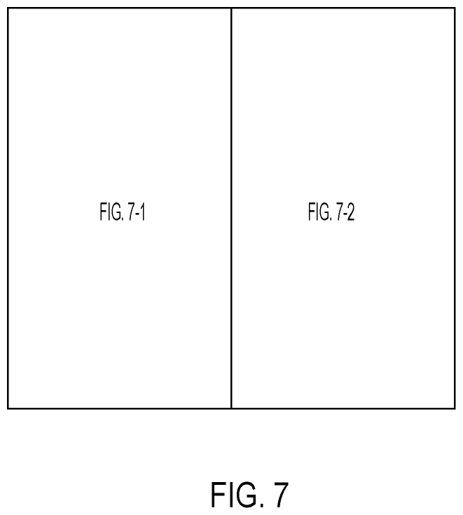

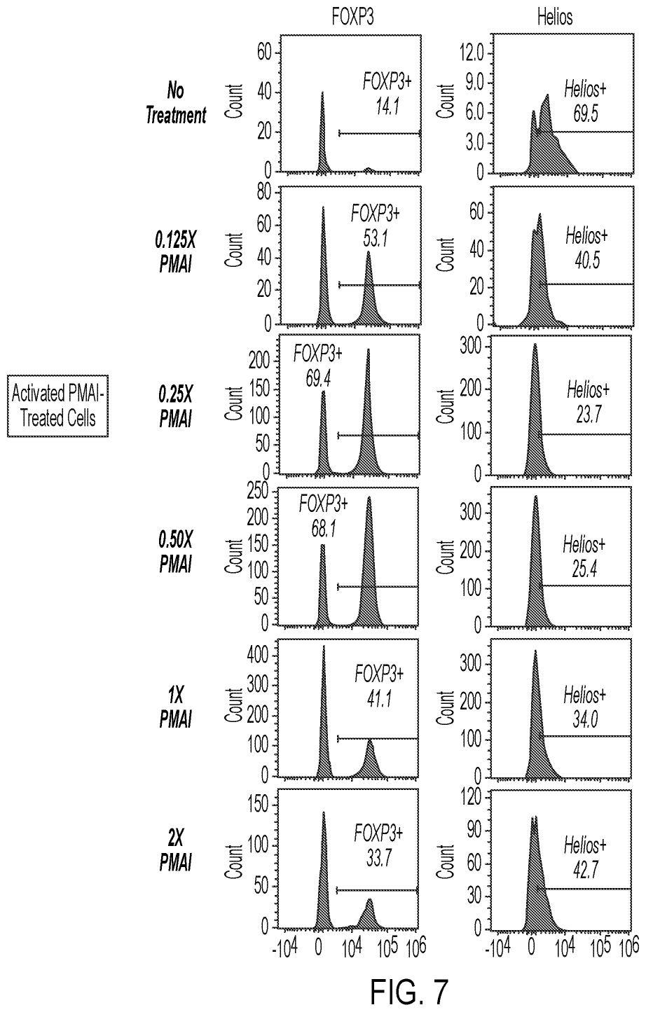

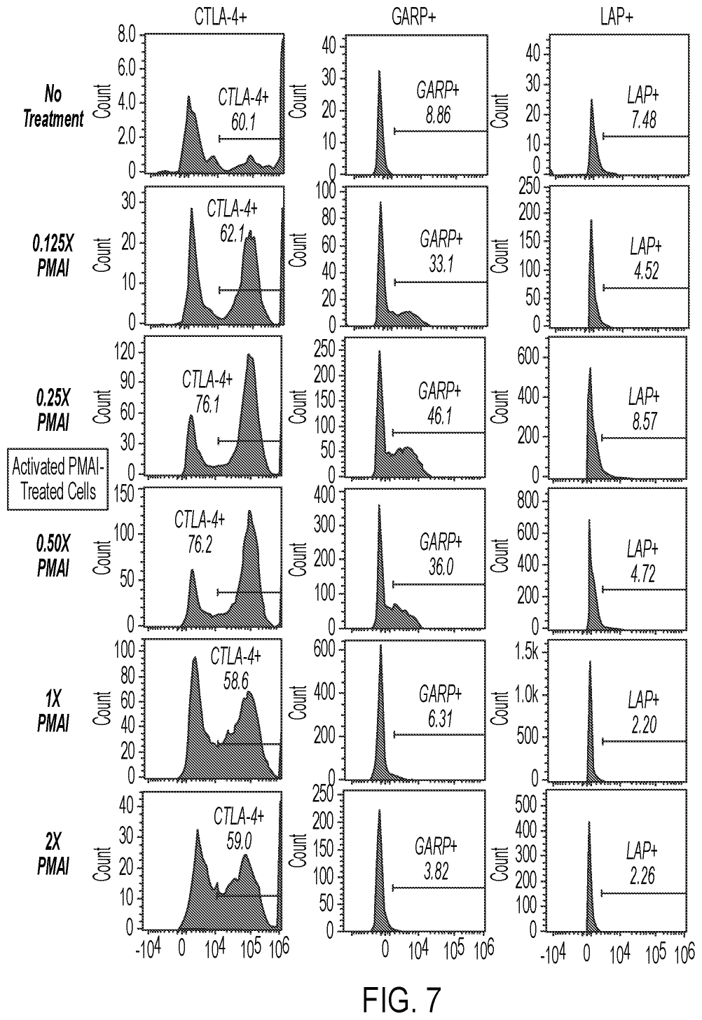

Flow cytometry analysis further showed the expression of Treg markers in activated PMAI-induced and TGF-β1-treated CD4sp T cells (iPSC-Tregs). The data show that FOXP3 and CTLA-4 were highly expressed on these cells, while Helios and LAP were expressed at moderate and low levels, respectively (FIG. 7). A moderate but distinct population of GARP⁺ cells were observed only in 0.125×, 0.25×, and 0.5×PMAI concentrations. Cells that were not treated with PMAI did not highly express these markers.

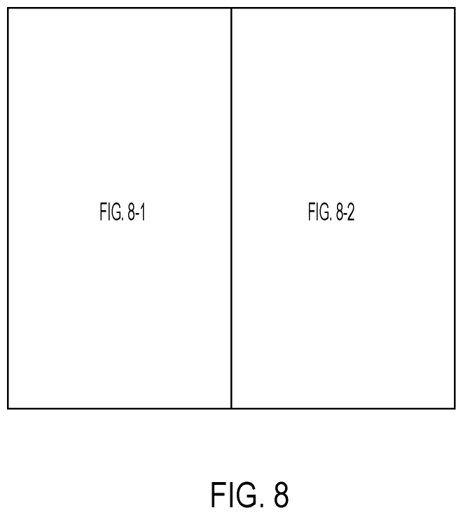

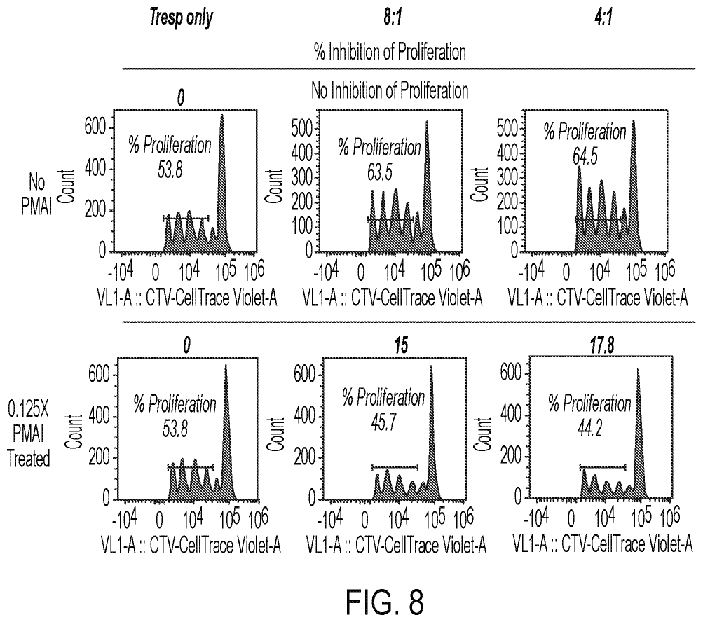

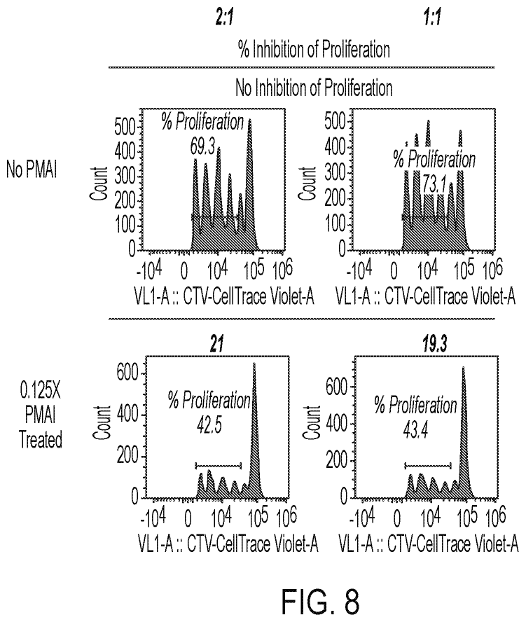

Flow cytometry analysis also showed the suppressive ability of 0.125×PMAI- and TGF-B1-treated iPSC-Tregs on the proliferation of responder T cells. At increasing numbers of responder T cells, iPSC-Tregs could suppress their proliferation between 15-21% (FIG. 8). Cells that were not treated with PMAI but treated with TGF-β1 and ATRA did not suppress T responder proliferation and appeared to stimulate target cell proliferation.

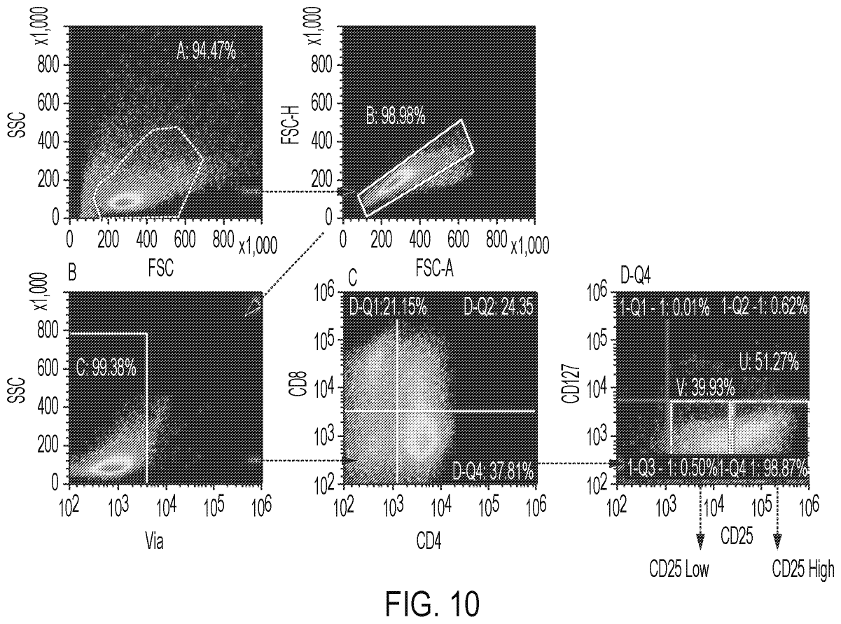

PMAI- and TGF-β1-treated iPSC-Tregs contained an impure population of cells. Thus, the cells were sorted to remove unwanted populations (CD8 single positive, CD4 and CD8 double positive, and CD4 and CD8 double negative cells) to obtain a population of cells that were predominately CD4 single positive, CD25 positive, CD127 negative/low, and FOXP3 positive. CD25 hi g h sorted cells contained more than 90% FOXP3⁺ cells, and CD25^{low} sorted cells contained a mixture of cells that were both FOXP3 positive and negative. These data show that gating on the fluorescence intensity of CD25 can yield the desired population of FOXP3⁺ cells (FIGS. 9 and 10).

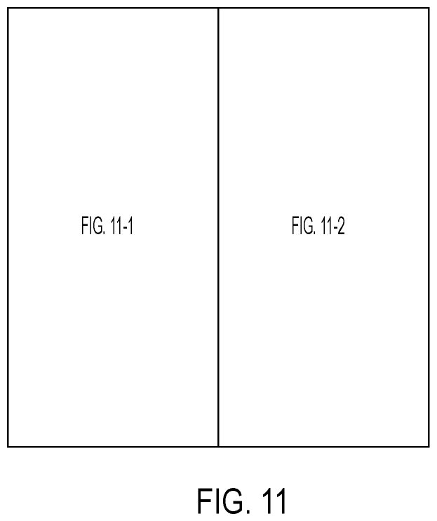

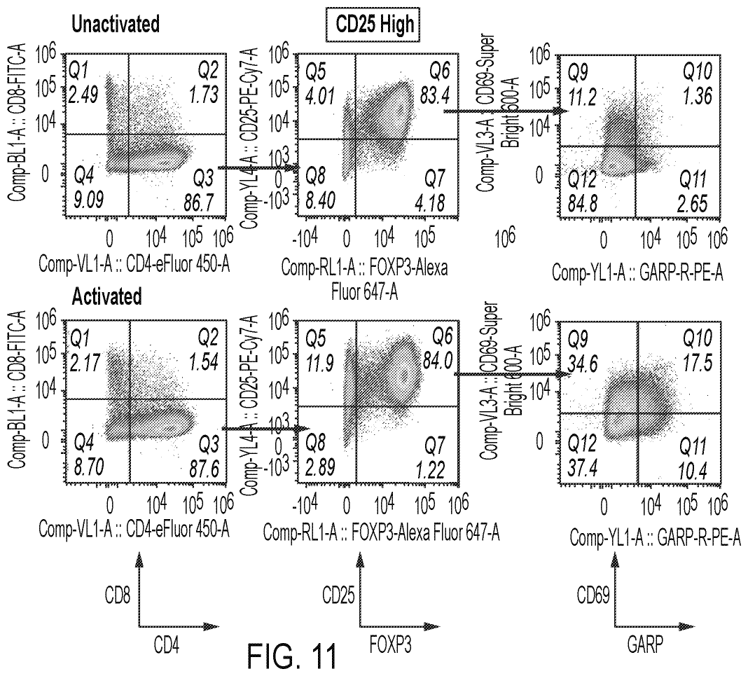

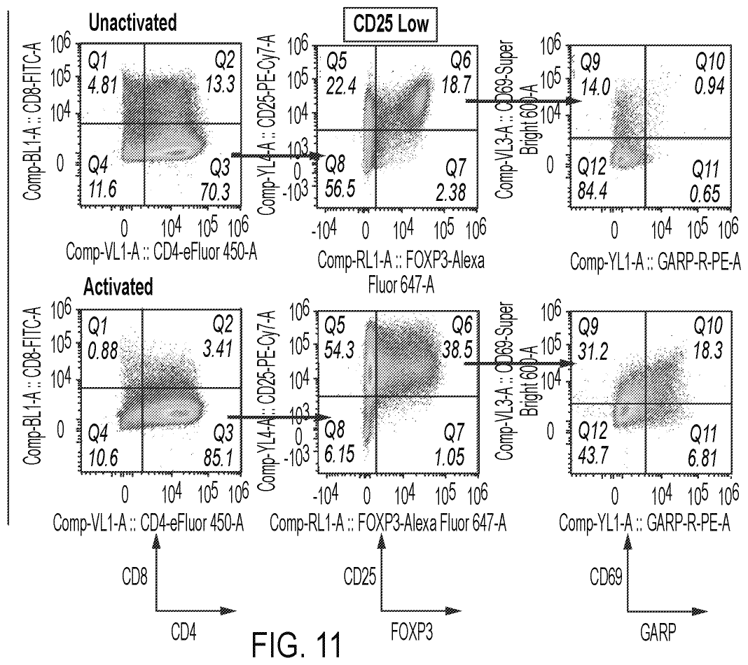

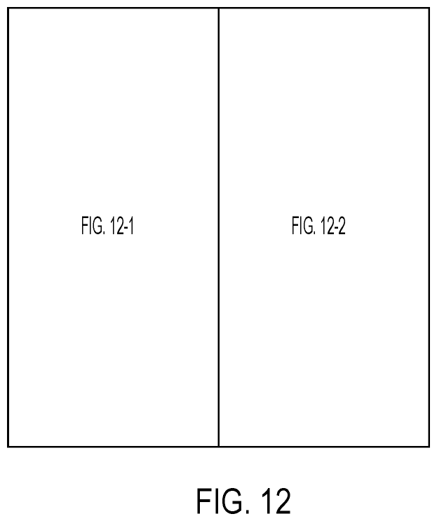

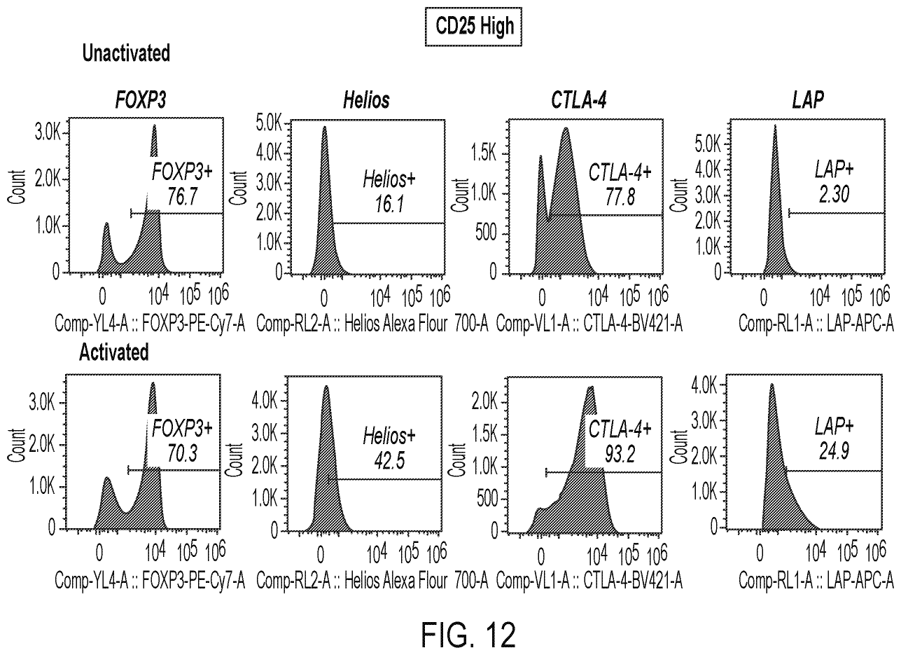

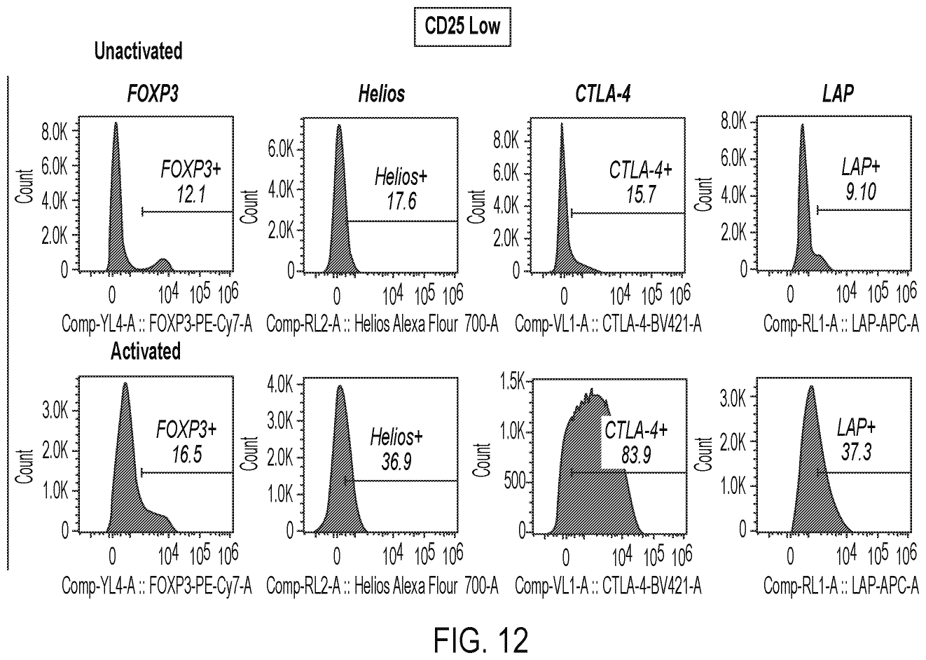

These sorted cells were activated to express markers of activated Tregs as determined by flow cytometry. CD25^{high} stimulated cells displayed elevated levels of CTLA-4 as well as moderate increases of Helios and LAP after activation as compared to unactivated cells. FOXP3 expression levels remained similar before and after activation. CD25^{high} stimulated cells also demonstrated an increase of CD69 and GARP expression and retention of Treg markers (CD4⁺CD25⁺ FOXP3⁺). CD25^{low} stimulated cells also demonstrated increased CTLA-4 expression as well as moderate increases of Helios and LAP expression after activation; however, FOXP3 levels remained much lower than CD25 hi g h cells despite activation (<18%). CD25^{low} stimulated cells also expressed CD69 and GARP upon activation (FIGS. 11 and 12).

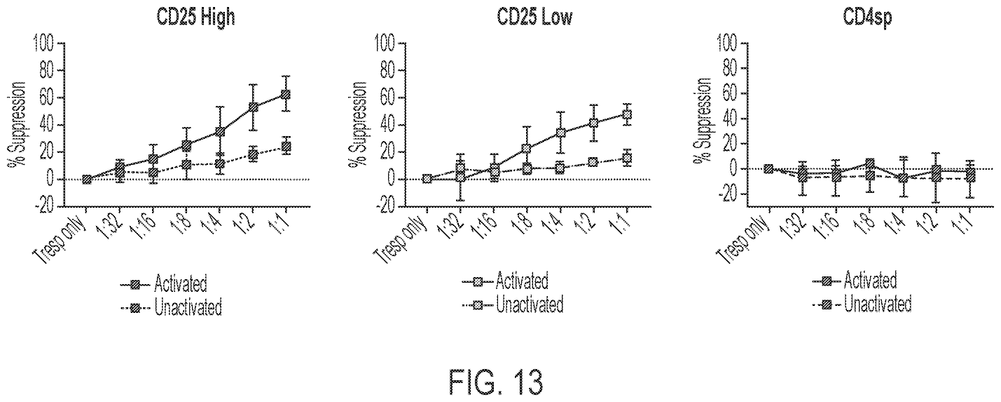

The sorted cells also showed the ability to suppress T cell proliferation. At an equal ratio of responder T cells to iPSC-Tregs, both the CD25^{high} and CD25^{low} iPSC-Tregs could suppress proliferation of responder T cells by 66-49% and 54-38% upon activation, respectively. PMAI-induced CD4sp cells, however, showed no suppressive effect on responder T proliferation (FIG. 13).

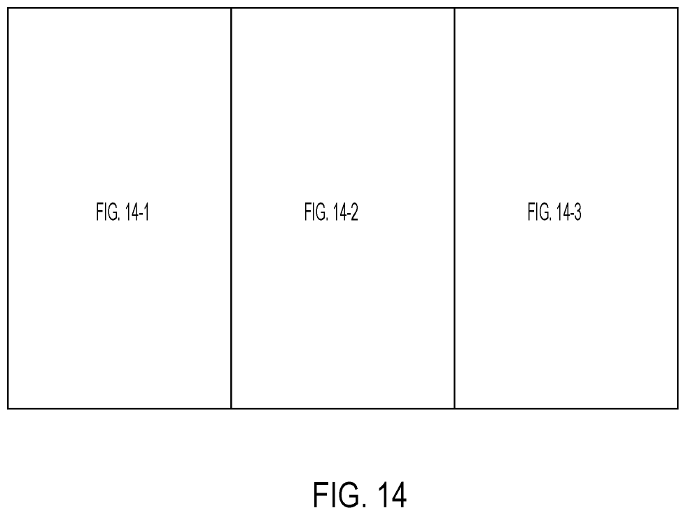

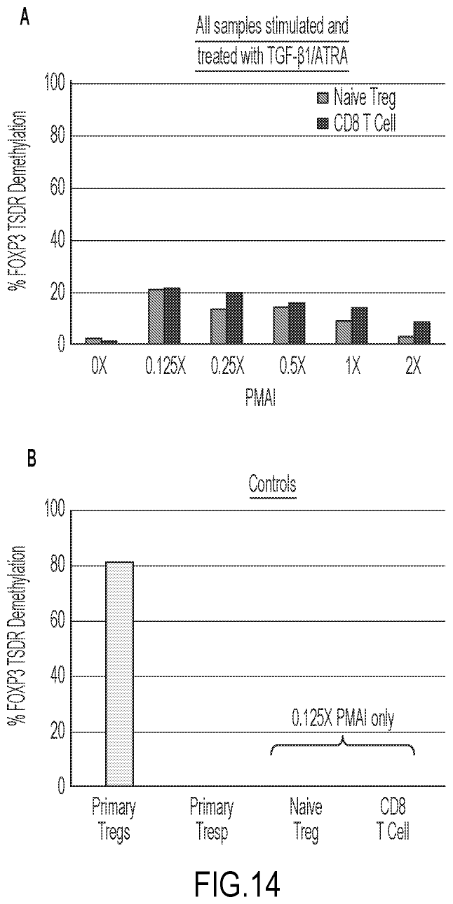

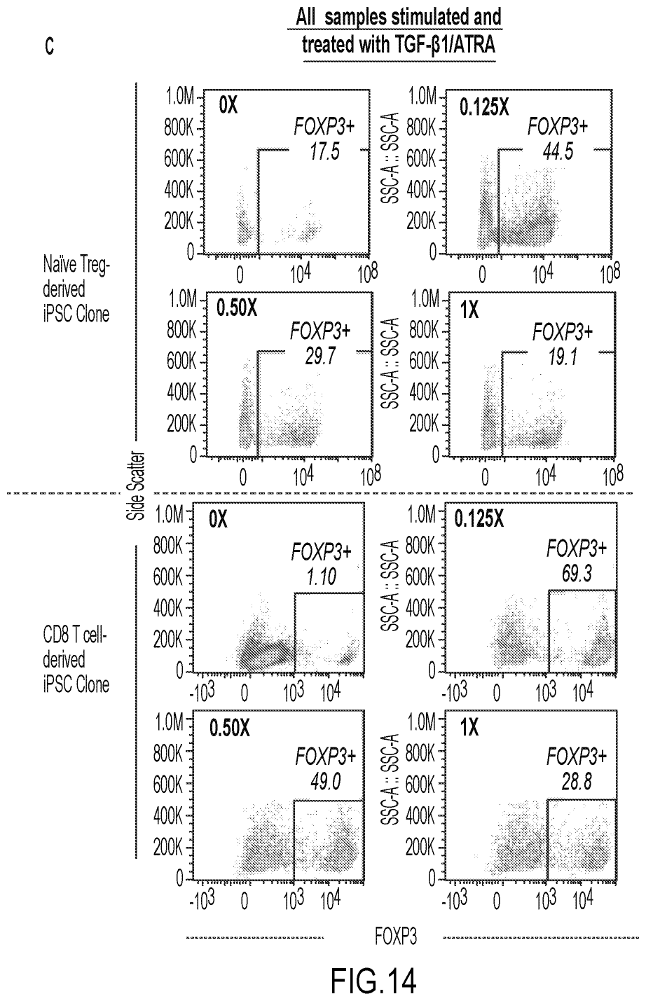

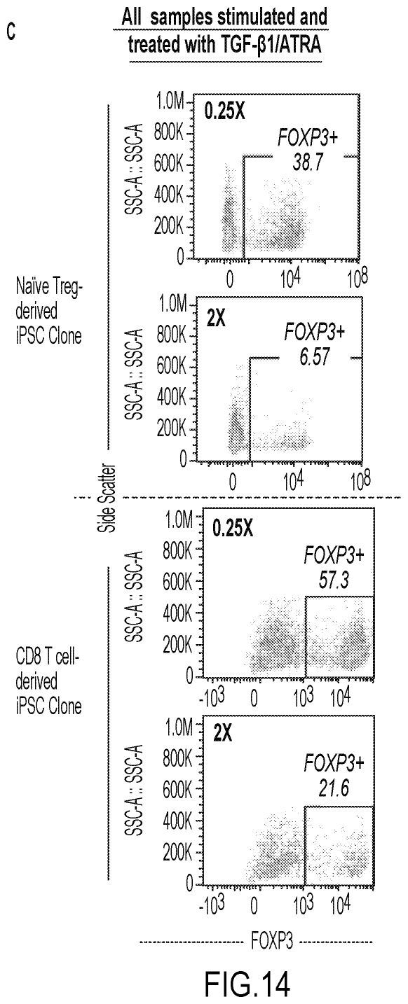

Analysis of FOXP3 Treg-specific demethylated region (TSDR) demethylation was performed on genomic DNA from PMAI-induced and stimulated TGF-β1 and ATRA-treated iPSC-Tregs. These cells showed an increase of demethylation at lower concentrations of PMAI. Cells that were not PMAI-induced but stimulated and treated with TGF-β1 and ATRA alone showed little to no demethylation at FOXP3 TSDR (FIG. 14, Panel A). Genomic DNA from primary Tregs showed more than 80% demethylation while primary Tresp and 0.125×PMAI-induced T cells were not highly methylated at TSDR (FIG. 14, Panel B). Analysis of FOXP3 expression by flow cytometry suggest that higher levels of FOXP3 correlated with a higher percentage of FOXP3 TSDR demethylation. In addition, cells treated with lower levels of PMAI generated more FOXP3⁺ cells after stimulation and TGF-β1 and ATRA treatment (FIG. 14, Panel C).

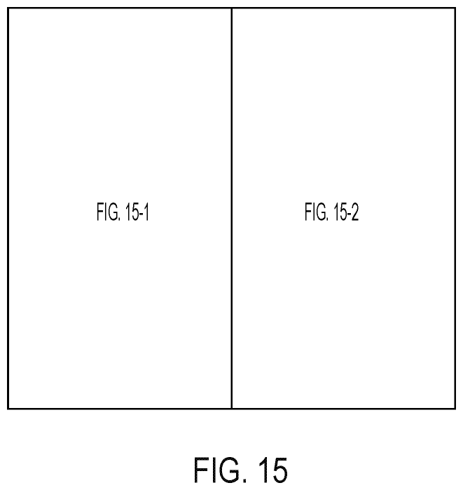

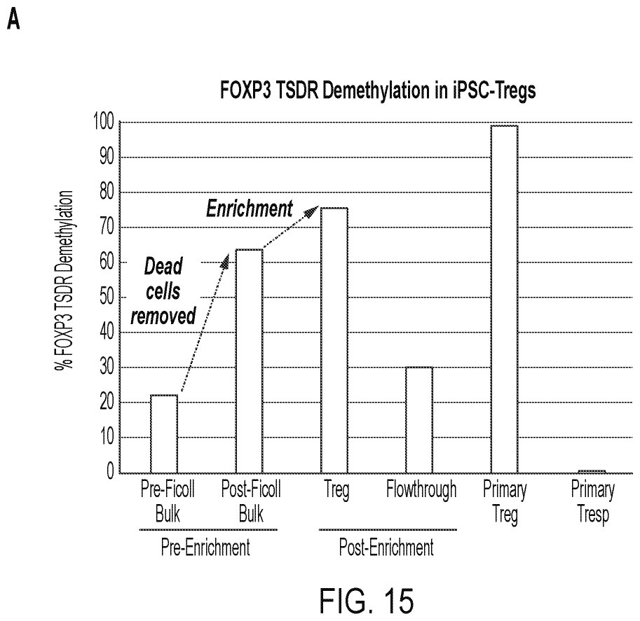

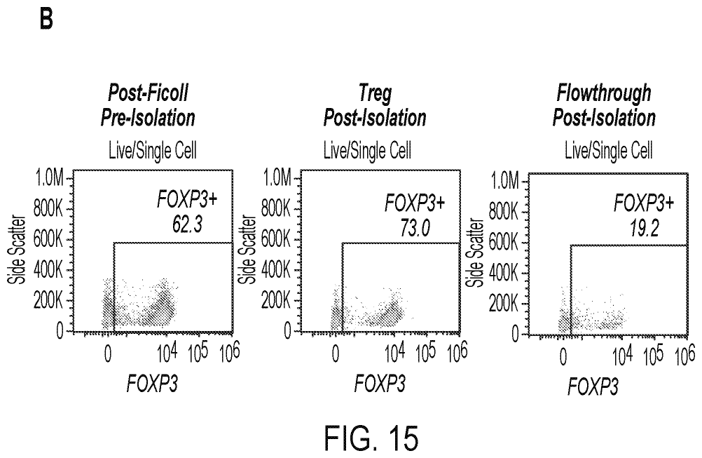

FOXP3 TSDR demethylation increased after dead cell removal (pre-Ficoll bulk vs. post-Ficoll bulk) and was further enhanced after Treg enrichment by immunomagnetic separation targeting CD25⁺CD4⁺CD127^{low} cells. The flowthrough or non-target population also contained TSDR demethylated cells that were likely not efficiently isolated or could indicate the presence of CD8 Tregs. Primary Tregs were highly TSDR demethylated and Tresp cells were highly methylated (FIG. 15, Panel A). Analysis of FOXP3 expression by flow cytometry suggest that higher levels of FOXP3 correlated with a higher percentage of FOXP3 TSDR demethylation (FIG. 15, Panel B).

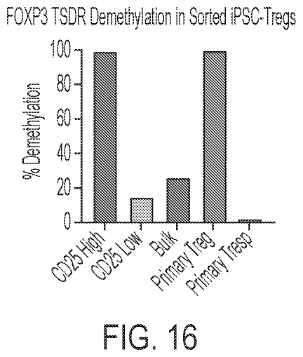

Sorting of the iPSC-Tregs to CD25^{high} and CD25^{low} populations further revealed the differences between the two populations. CD25^{high} sorted iPSC-Tregs were highly demethylated at FOXP3 TSDR (>90%) compared to CD25^{low} sorted iPSC-Tregs (˜14%). Primary Tregs were also highly demethylated while primary T responder cells were highly methylated at FOXP3 TSDR (FIG. 16).

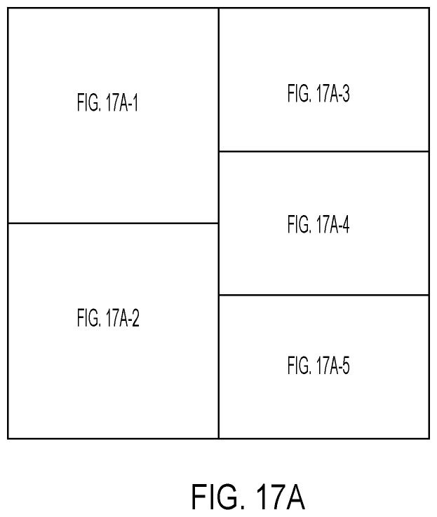

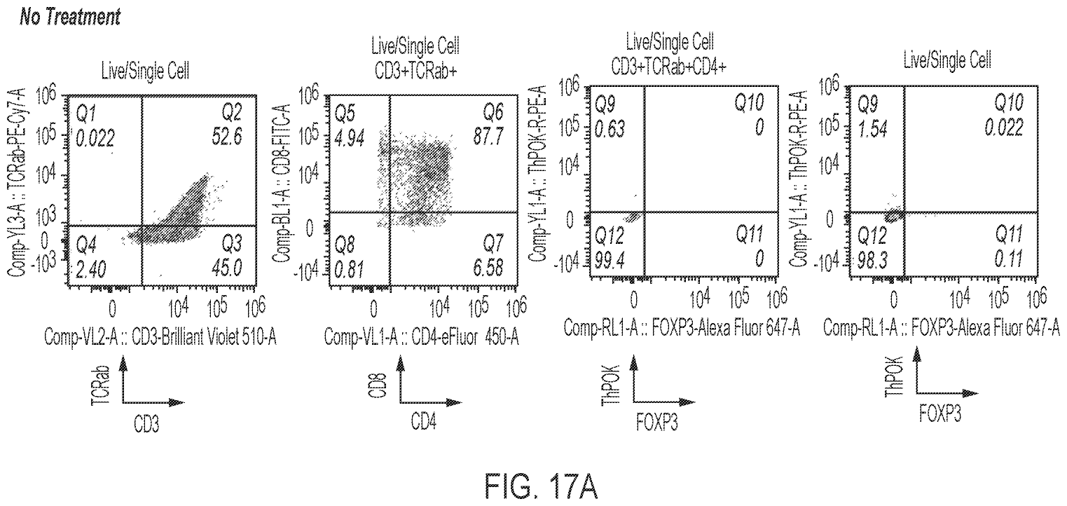

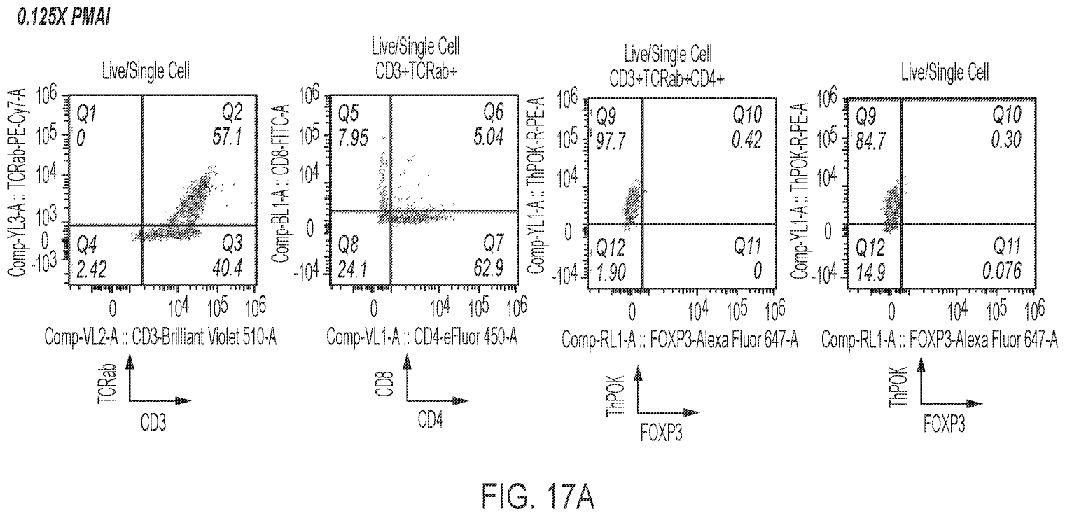

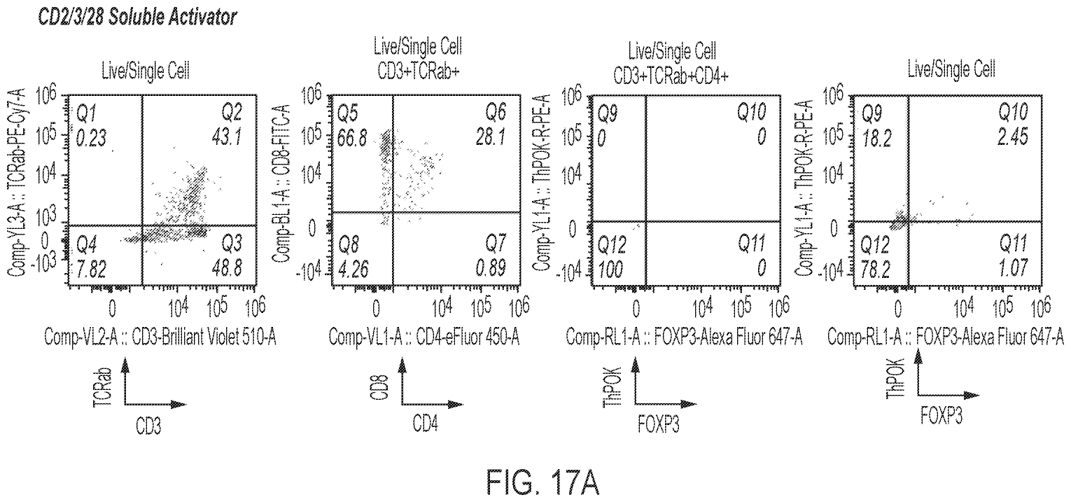

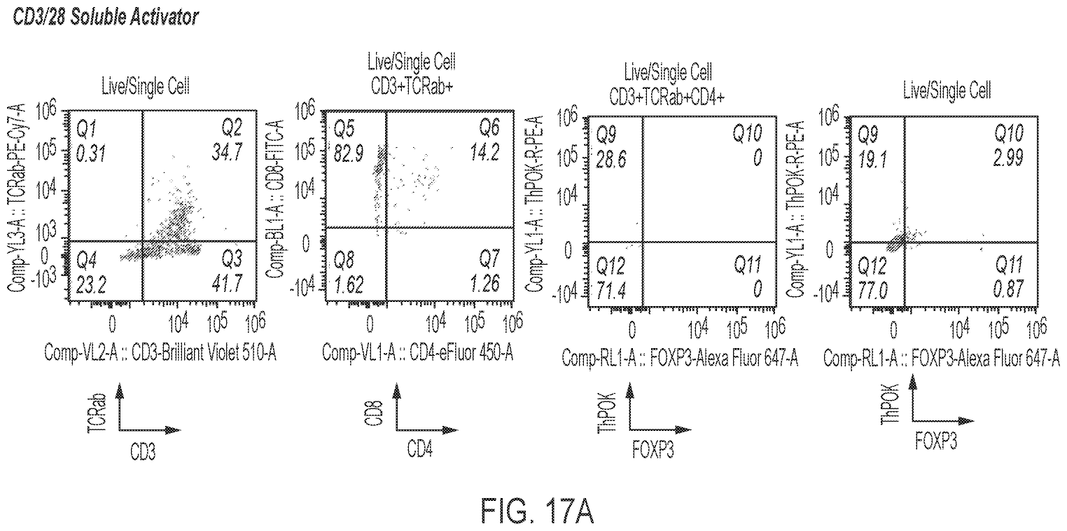

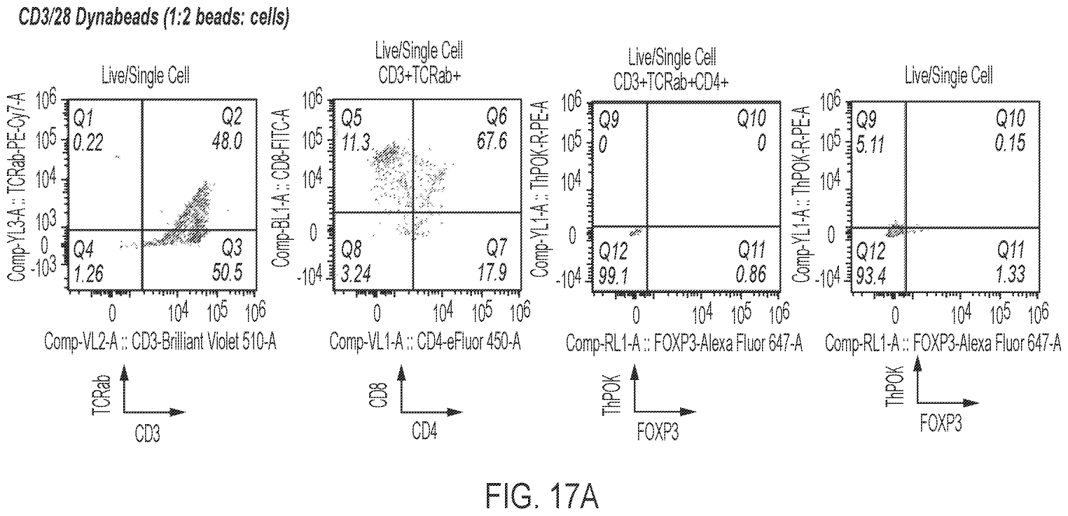

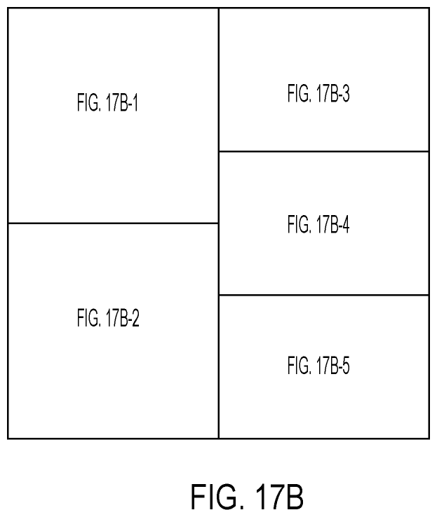

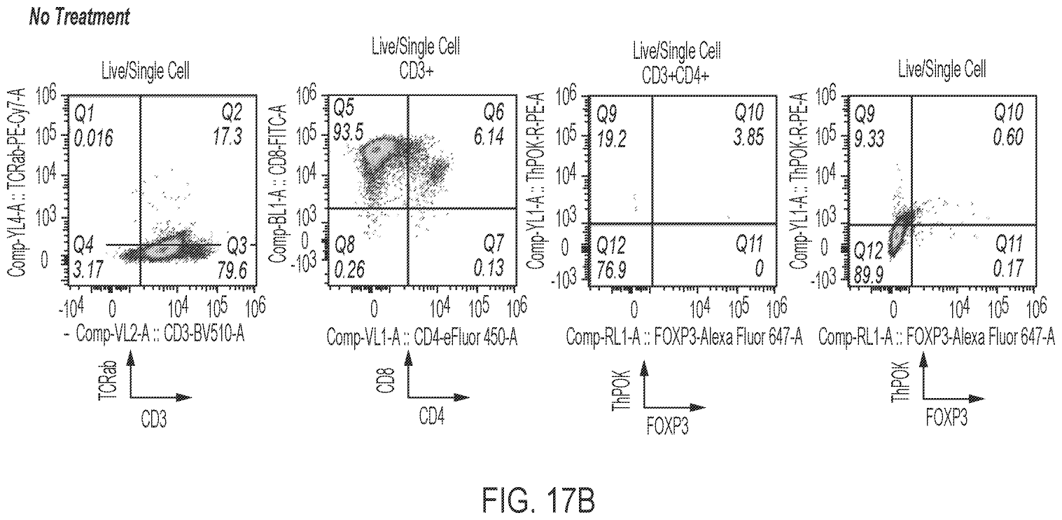

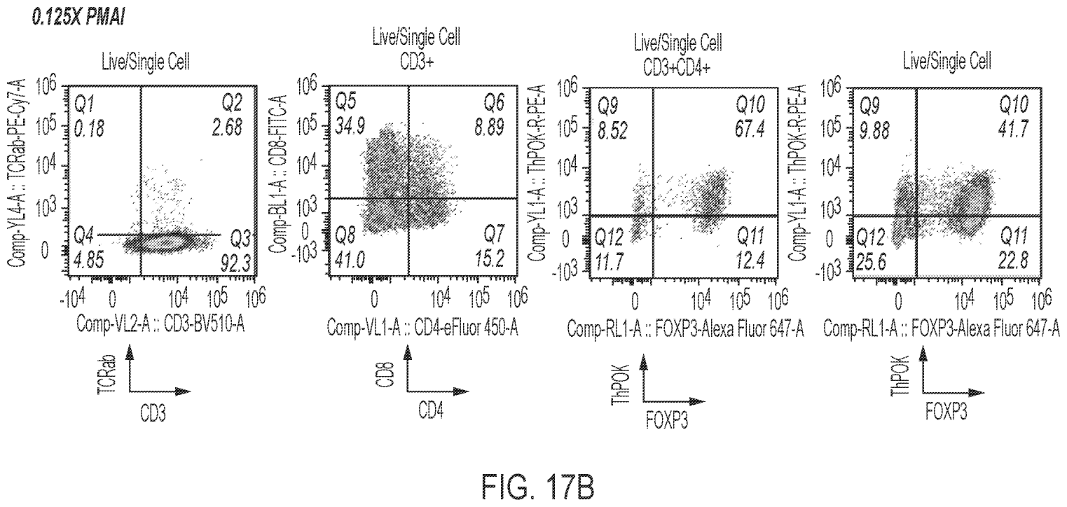

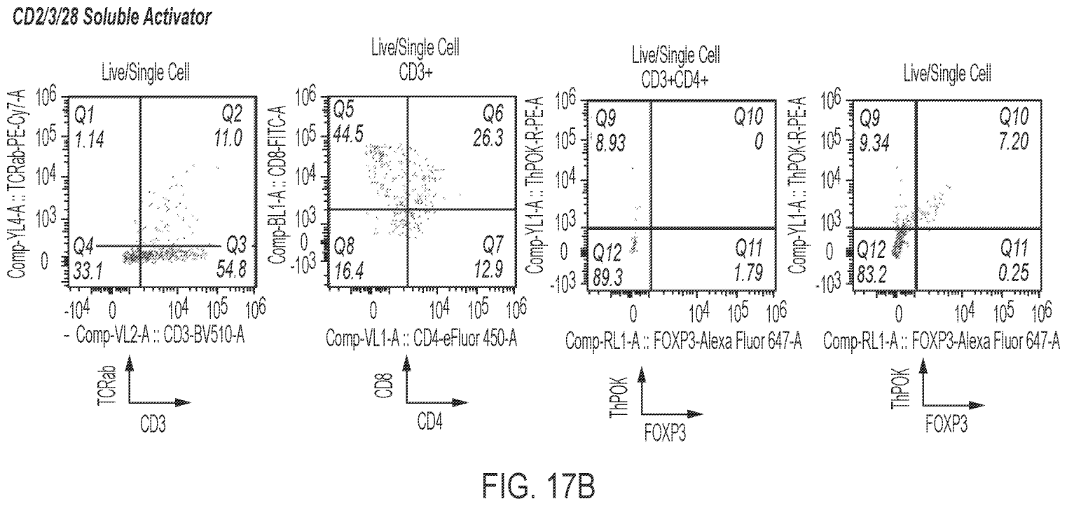

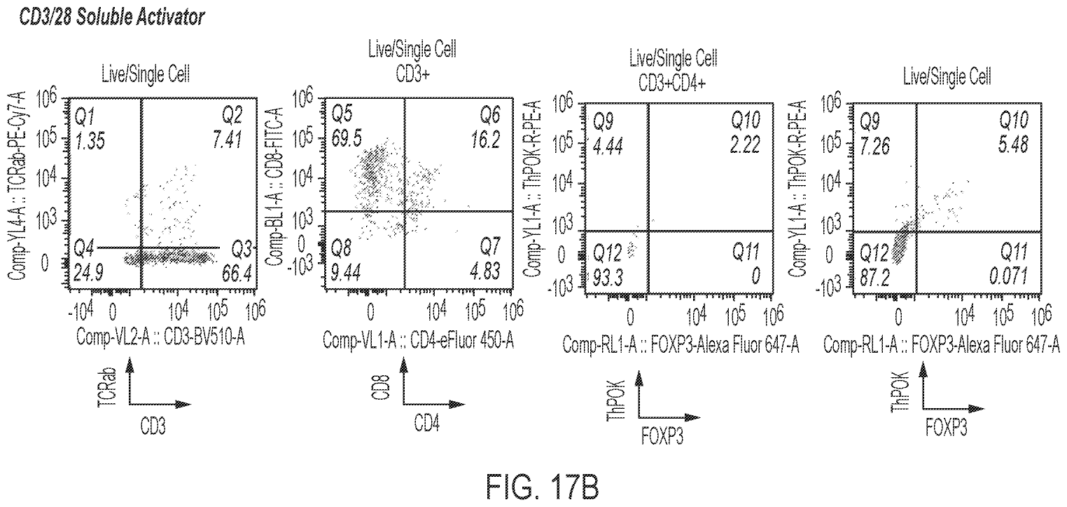

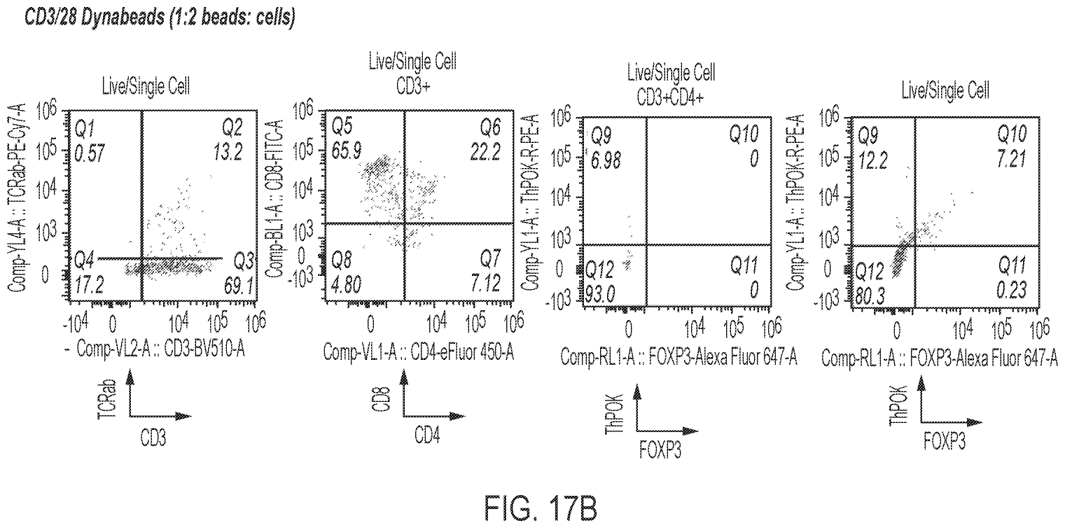

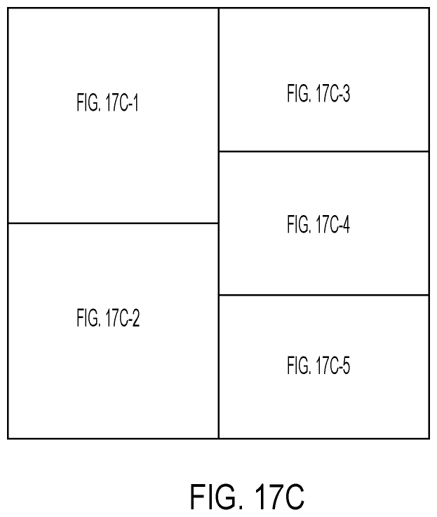

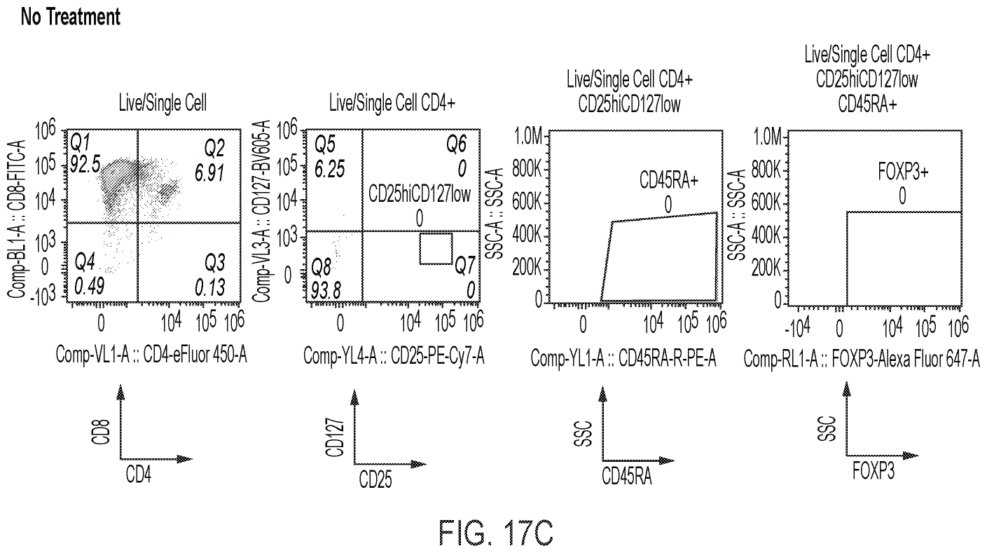

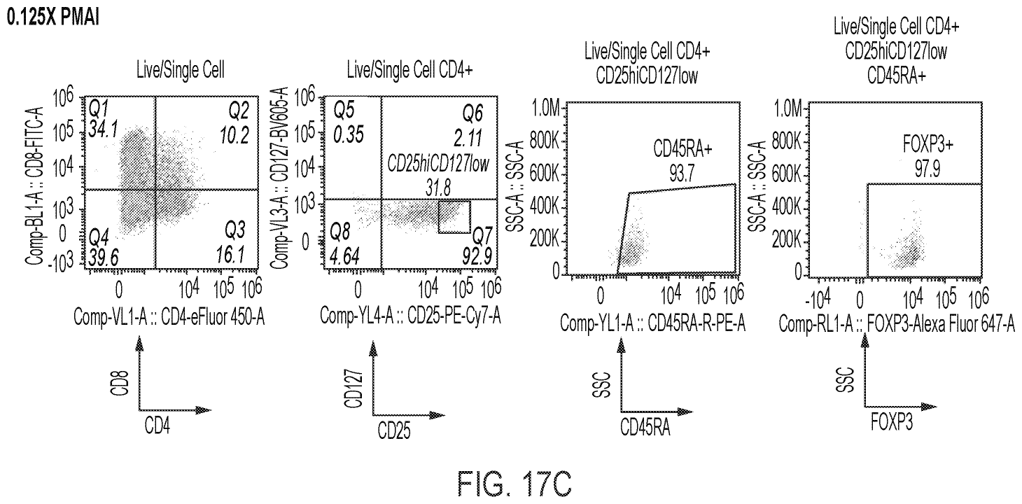

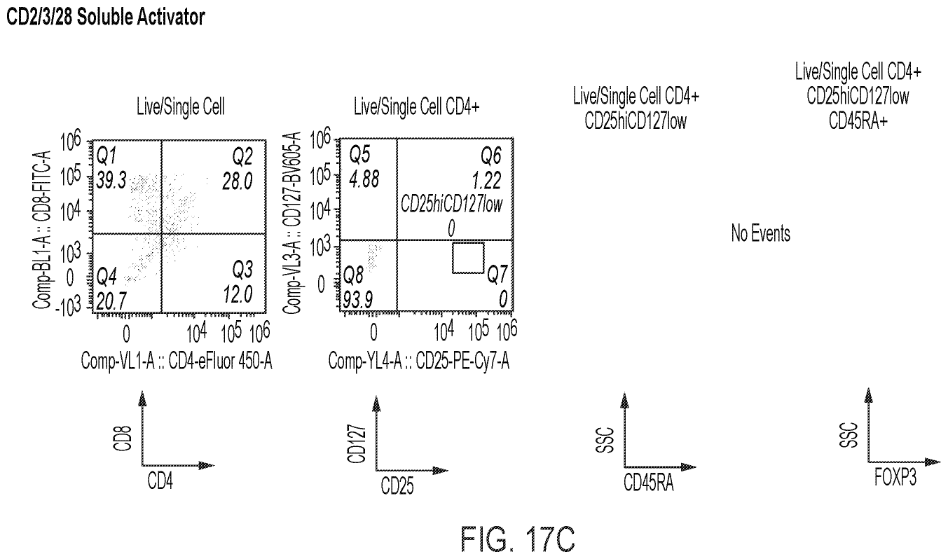

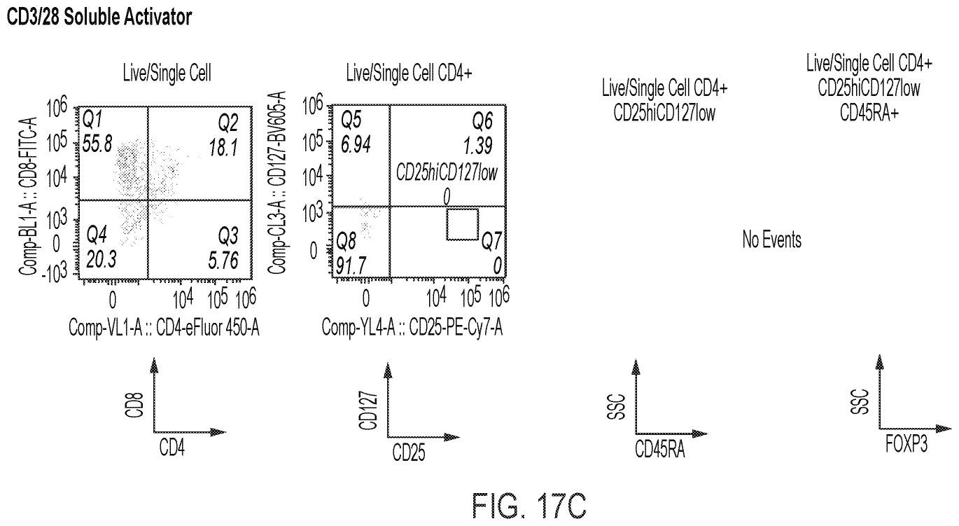

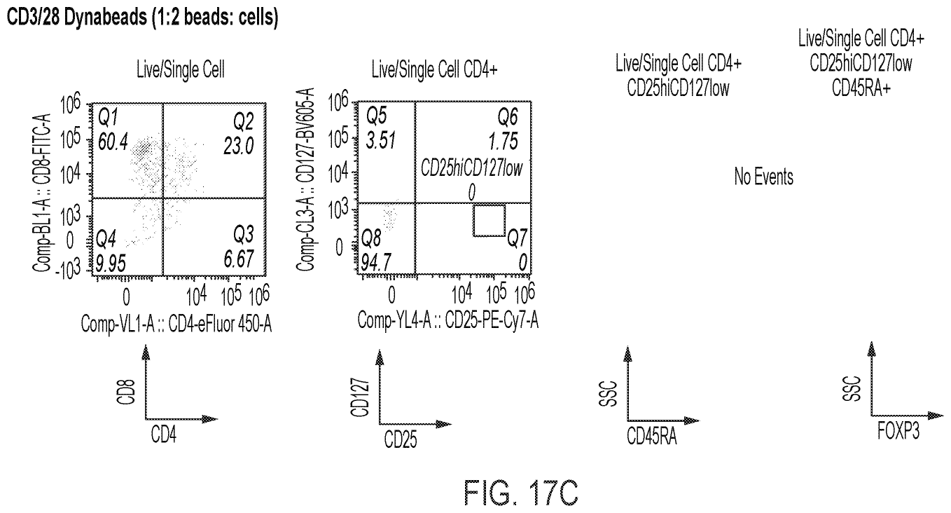

Commonly used T cell stimulating reagents were evaluated for their ability to generate CD4sp T cells from iPSC-derived DP T cells. Soluble tetrameric antibody complexes that bind either CD3 and CD28 or CD3, CD28, and CD2 (StemCell Technologies) were tested alongside CD3/CD28 Dynabeads (Thermo Fisher), magnetic beads coupled to anti-CD3 and anti-CD28 antibodies. Cells with no PMAI treatment or treated with 0.125× PMAI were used as controls. Eight days after stimulating with various reagents, only 0.125× PMAI-treated cells were able to generate a distinct population of CD4sp cells that were also ThPOK+ (FIG. 17A). Stimulated cells were then treated with TGF-β1 and ATRA. Only PMAI-stimulated cells generated CD4sp cells that also expressed ThPOK and FOXP3 (FIG. 17B). In addition, only PMAI-stimulated cells generated naïve Tregs that were CD25^{high}CD127^{low}CD45RA⁺FOXP3⁺ (FIG. 17C).

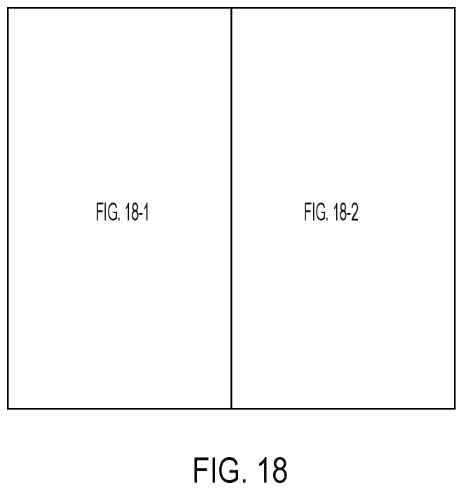

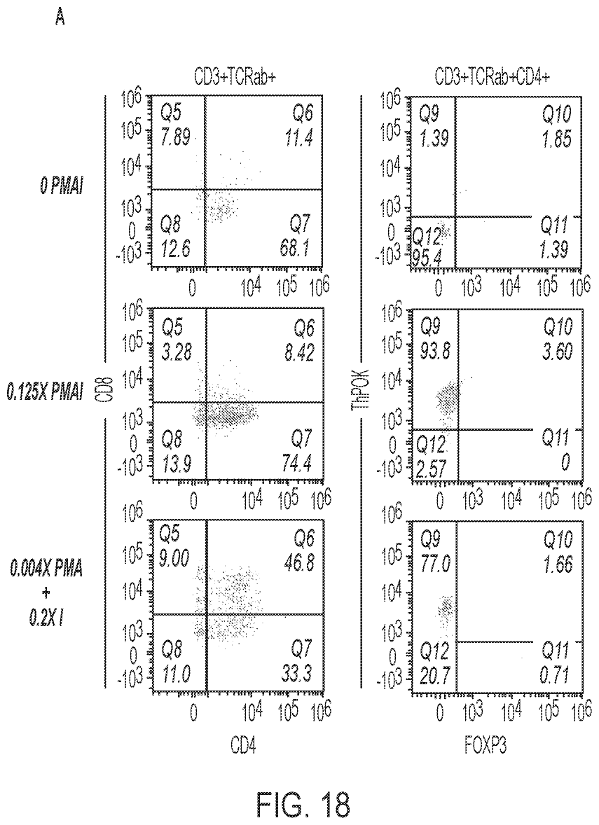

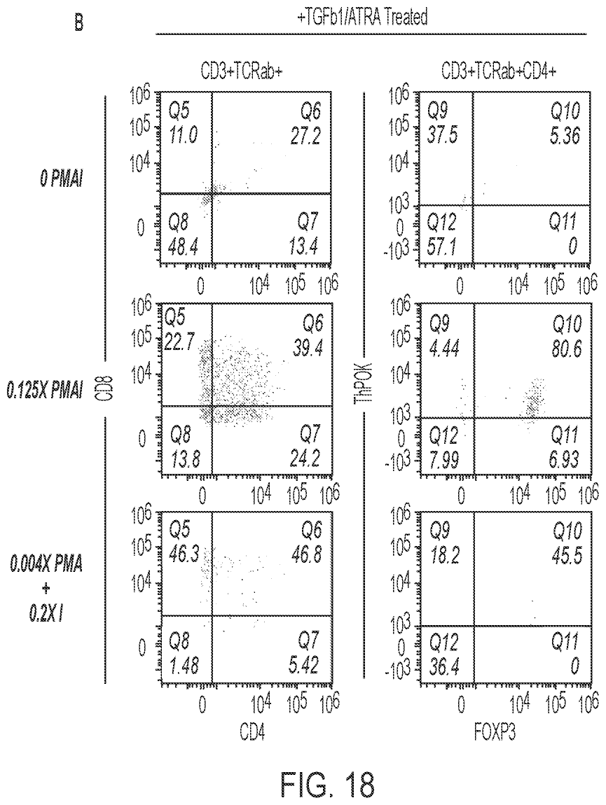

A combination of 0.004×PMA and 0.2× ionomycin were also evaluated for its ability to promote generation of CD4sp T cells (FIG. 18). This concentration of PMAI generated approximately 33% CD4sp T cells as compared to approximately 74% CD4sp T cells generated from 0.125×PMAI. 0.125×PMAI-induced CD4sp T cells were almost all ThPOK⁺ compared to 0.004×PMA and 0.2× ionomycin-induced CD4sp T cells, which were only approximately 77% positive. Addition of TGF-β1 and ATRA to 0.004×PMA and 0.2×ionomycin-induced cells converted the cells to CD8⁺ and DP T cells. Addition of TGF-β1 and ATRA to 0.125×-induced PMAI retained a population of CD4sp T cells that were 80% ThPOK⁺FOXP3⁺.

### Example 4: Integrating Transgene into the AAVS1 Gene Locus of iPSCs

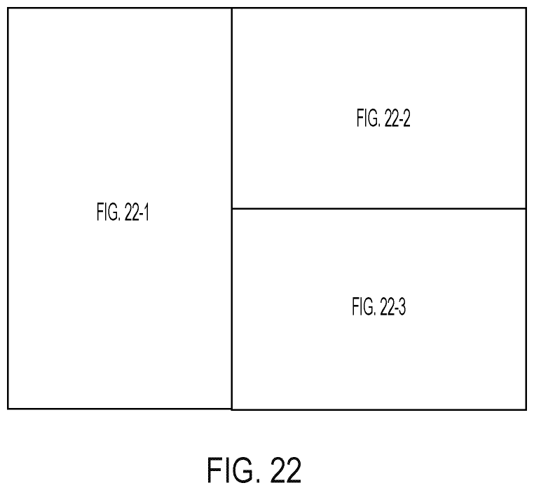

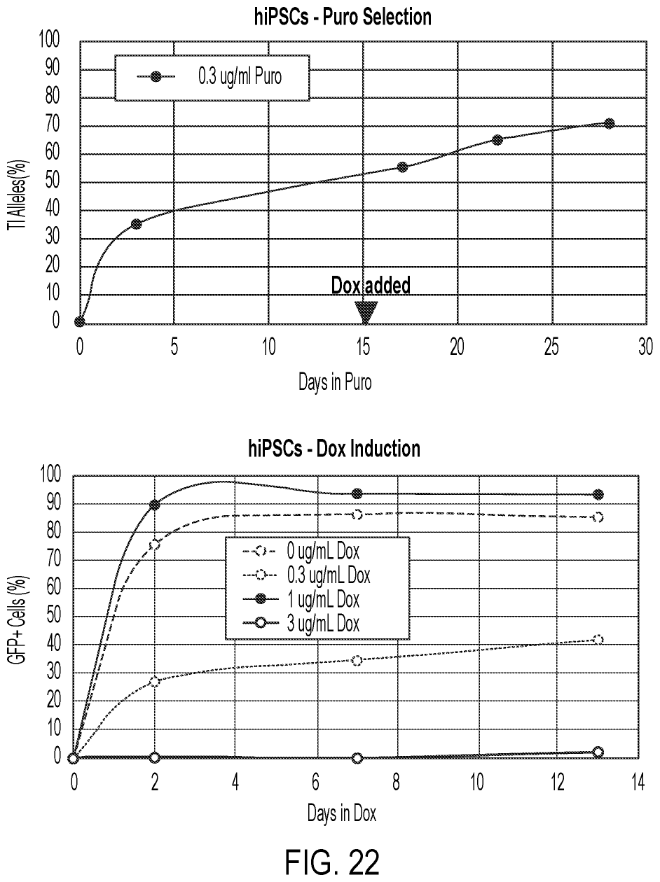

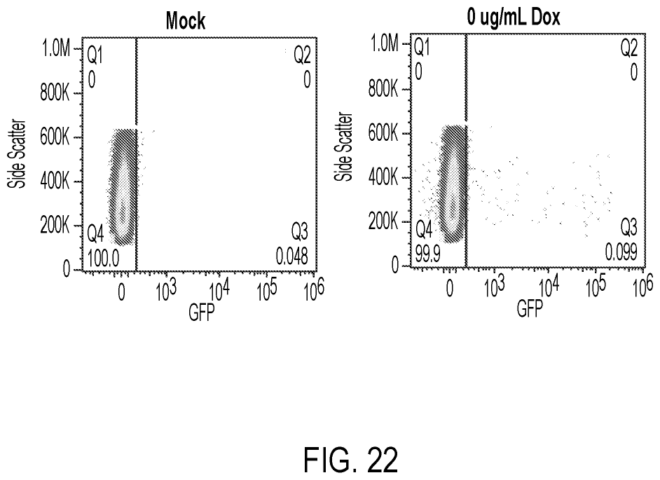

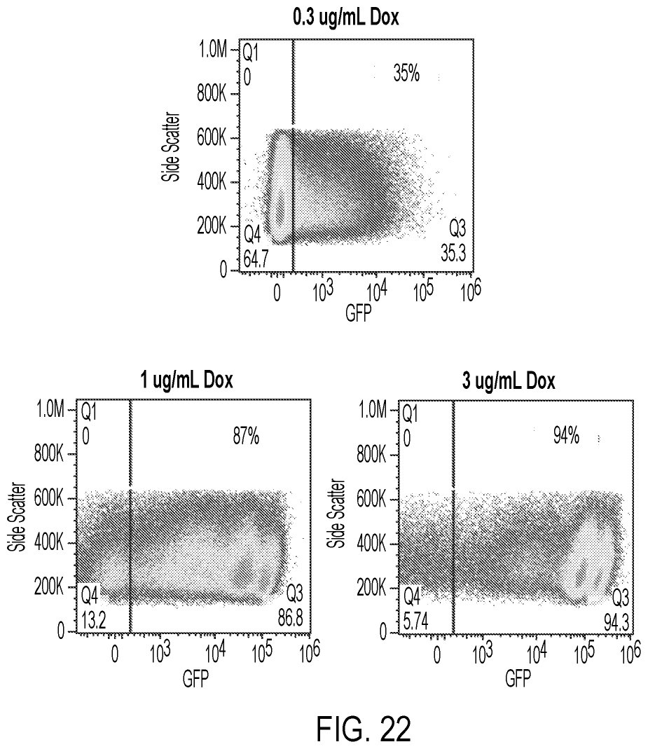

This Example describes an experiment in which a green fluorescent protein expression cassette was integrated into the AAVS1 gene locus as illustrated in FIG. 21. AAVS1 ZFN mRNA and donor plasmid were delivered into iPSCs via electroporation on Day −7. One week later (Day 0), puromycin was added (0.3 μg/mL) to the tissue culture to begin positive selection for cells having undergone targeted integration. Doxycycline was added at Day 15 and maintained in culture at 3 different doses (0.3, 1, and 3 μg/mL) to induce expression of the dox-inducible GFP expression cassette. Control cells did not have added doxycycline in culture. Cells were maintained in the presence of doxycycline for 13 days. During this period, the 3 μg/mL dose of doxycycline yielded the highest level of inducible GFP transgene expression (94%; FIG. 22). This high level of expression was sustained while doxycycline was present in culture. Cells were maintained in puromycin as well as doxycycline from Day 15-28 and were further positively selected (increase from ˜50% to ˜70% of alleles with targeted integration).

### Example 5: Skewing Differentiation of iPSCs Towards CD4⁺ Treg Lineage

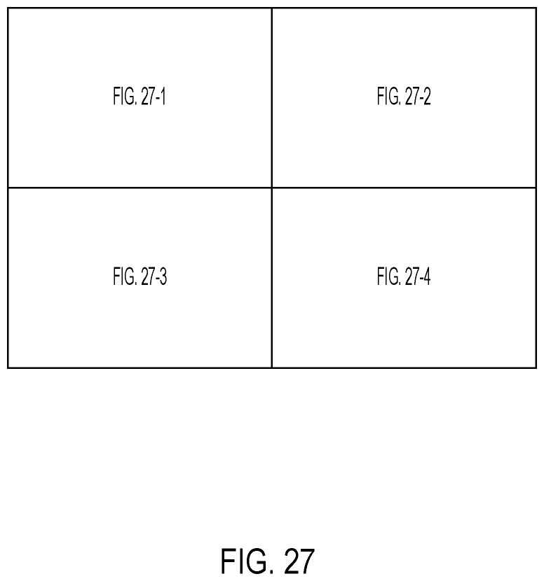

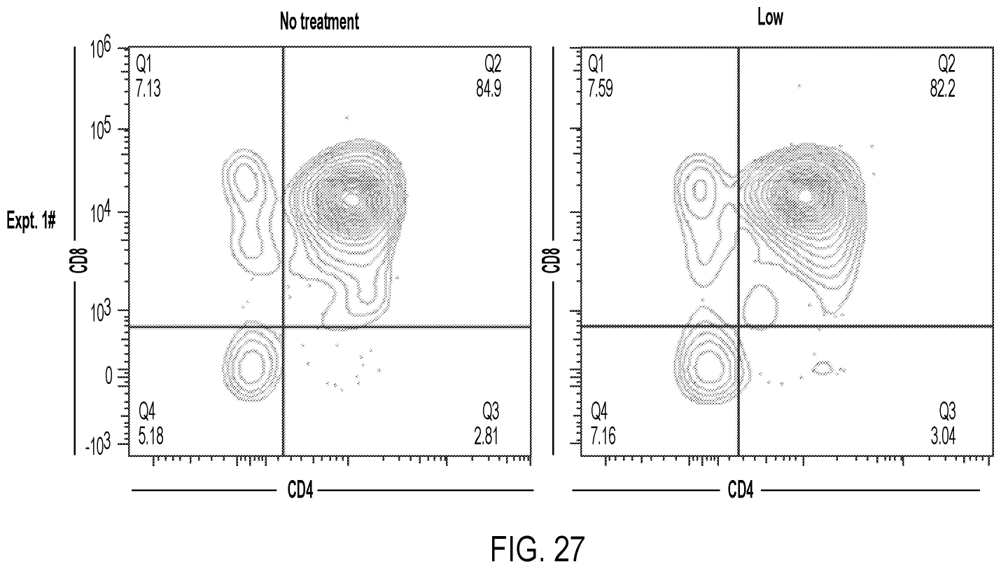

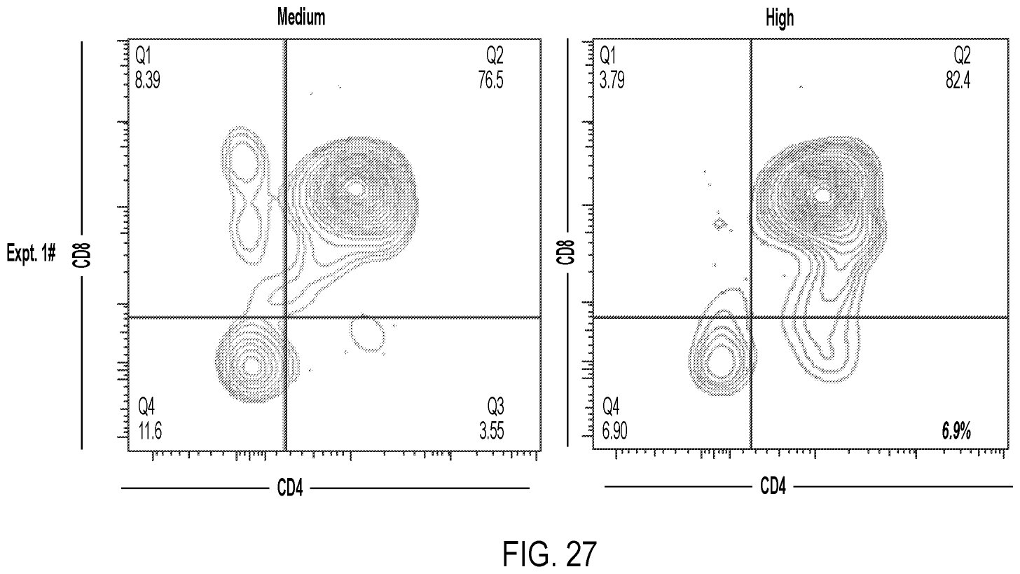

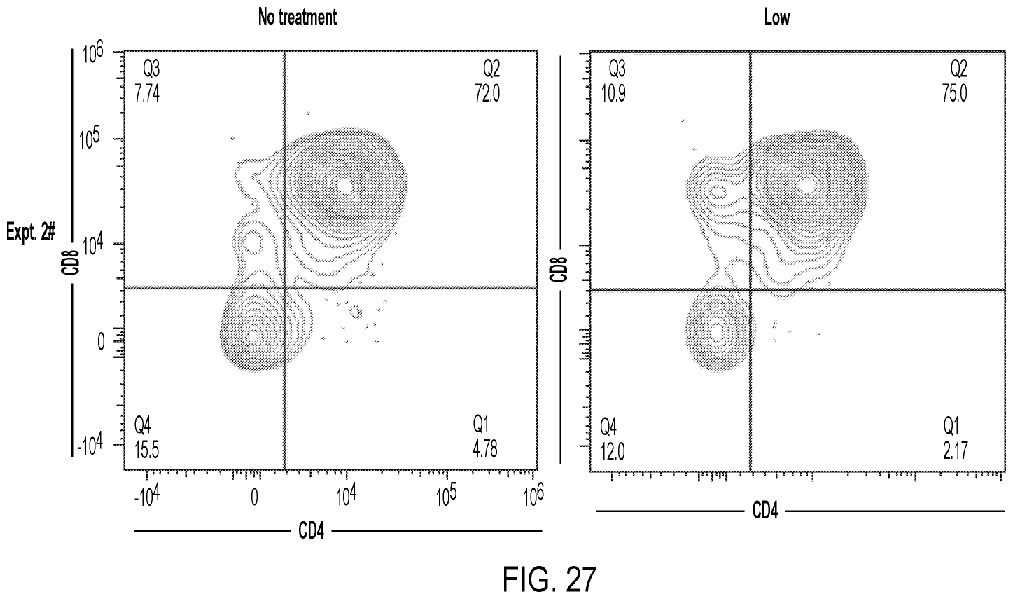

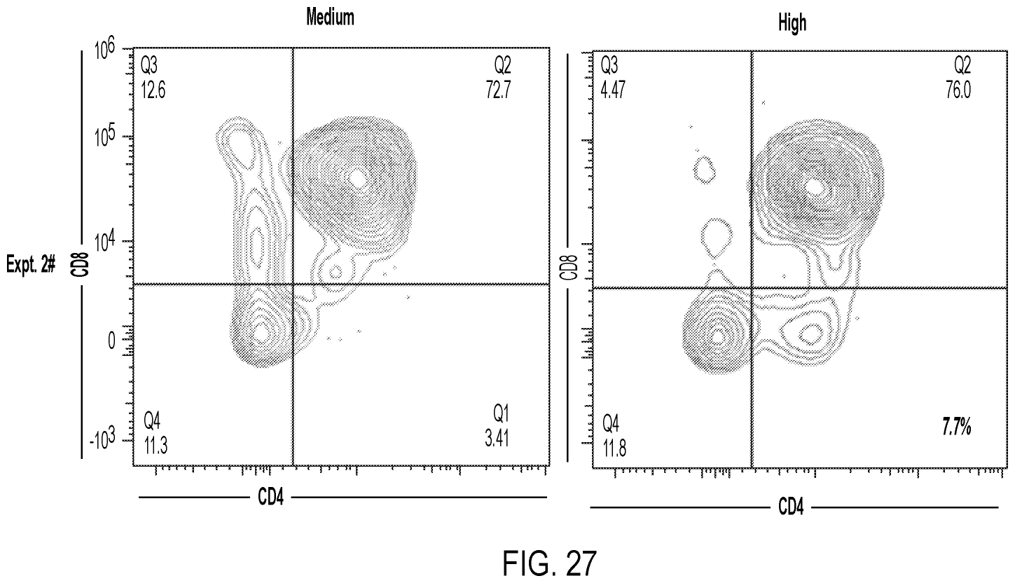

To skew the differentiation of iPSCs toward becoming CD4⁺ T cells and ultimately Treg cells, the stem cells were subjected to blockage of signaling through IL-7 receptor by using an antibody targeting the alpha unit of the IL-7 receptor (IL-7Ra). Anti-IL-7Ra antibody was added to the cell culture media at increasing concentrations during the later stages of T cell development. Two duplicate experiments (Expt. 1 and Expt. 2) both show that addition of anti-IL-7Ra antibody increased the percentage of CD4⁺ single positive cells (bottom right quadrants), reaching 6.9% (Expt. 1) or 7.7% (Expt. #2), as compared to 2.81% or 4.78% for untreated cells, while reducing the percentage of CD8⁺ single positive cells (top left quadrants) (FIG. 27).

1. A method of obtaining a population of cells enriched for CD4 single positive T cells, comprising:
providing a starting population of CD4⁺CD8⁺ T cells,
culturing the starting population of cells in a medium comprising phorbol 12-myristate 13-acetate (PMA) and ionomycin, thereby obtaining a population of cells enriched for CD4 single positive T cells.

2. The method of claim 1, wherein the culture medium contains 0.00625 to 0.1 μg/ml PMA and 0.125 to 2 μg/ml of ionomycin.

3. The method of claim 1 or 2, wherein the weight ratio of PMA to ionomycin is 1:10 to 1:1000, optionally 1:20.

4. The method of claim 3, wherein the culture medium comprises 0.00625 μg/ml PMA and 0.125 μg/ml of ionomycin.

5. The method of any one of claims 1-4, wherein the cells are cultured in the medium for about one to five days.

6. The method of any one of claims 1-5, wherein the CD4 single positive T cells are immature CD4⁺ T cells, optionally wherein the T cells express ThPOK.

7. The method of claim 6, wherein the immature CD4⁺ T cells are effector T (Teff) cells, optionally wherein the Teff cells are CD25^{low}.

8. The method of any one of claims 1-6, further comprising
culturing the CD4 single positive T cells in a second medium comprising TGF-β and all trans-retinoic acid (ATRA), thereby obtaining a population of cells enriched for CD4⁺ regulatory T (Treg) cells.

9. The method of claim 8, wherein the second medium further comprises IL-2, an anti-CD2 antibody, an anti-CD3 antibody, and an anti-CD28 antibody.

10. The method of claim 8, wherein the second medium comprises a T cell-specific medium, and one or more of TGF-0, ATRA, IL-2, an anti-CD2 antibody, an anti-CD3 antibody, and an anti-CD28 antibody.

11. The method of any one of claims 8-10, wherein the CD4 single positive T cells are cultured in the second medium for about 5-10 days.

12. The method of any one of claims 1-11, further comprising isolating the CD4 single positive Teff or Treg cells from the tissue culture by fluorescence-activated cell sorting (FACS) or magnetic-activated cell sorting (MACS).

13. The method of any one of claims 1-11, further comprising isolating the CD4 single positive Teff or CD4⁺CD25⁺CD127^{low} Treg cells from the tissue culture by fluorescence-activated cell sorting (FACS).

14. The method of any one of claims 1-11, further comprising isolating the CD4⁺CD25^{high} CD127^{low} and CD4⁺CD25^{low} CD127^{low} Treg cells from the tissue culture by FACS.

15. The method of any one of claims 1-14, wherein the starting population of CD4⁺CD8⁺ T cells are derived from human induced pluripotent stem cells (iPSCs).

16. The method of claim 15, wherein the iPSCs are reprogrammed from T cells.

17. The method of claim 15 or 16, wherein the iPSCs comprise a heterologous sequence in the genome,
wherein the heterologous sequence comprises a transgene encoding a lineage commitment factor, and
wherein the lineage commitment factor
(i) promotes the differentiation of the iPSCs to CD4⁺ Teff cells or
(ii) promotes the differentiation of the iPSCs to CD4⁺ Treg cells and/or promotes the maintenance of the phenotype of the CD4⁺ Treg cells.

18. The method of claim 17, wherein the heterologous sequence is integrated into a T cell specific gene locus such that expression of the transgene is under the control of transcription-regulatory elements in the gene locus.

19. The method of claim 17 or 18, wherein the transgene comprises a coding sequence for an additional polypeptide, wherein the coding sequence for the lineage commitment factor and the coding sequence for the additional polypeptide are separated by an in-frame coding sequence for a self-cleaving peptide or by an internal ribosome entry site (IRES).

20. The method of claim 19, wherein the additional polypeptide is another lineage commitment factor, a therapeutic protein, or a chimeric antigen receptor.

21. The method of any one of claims 17-20, wherein the heterologous sequence is integrated into an exon in the T cell specific gene locus and comprises:
an internal ribosome entry site (IRES) immediately upstream of the transgene; or
a second coding sequence for a self-cleaving peptide immediately upstream of and in-frame with the transgene.

22. The method of claim 21, wherein the heterologous sequence further comprises, immediately upstream of the IRES or the second coding sequence for a self-cleaving peptide, a nucleotide sequence comprising all the exonic sequences of the T cell specific gene locus that are downstream of the integration site, such that the T cell specific gene locus remains able to express an intact T cell specific gene product.

23. The method of any one of claims 18-22, wherein the T cell specific gene locus is a T cell receptor alpha constant (TRAC) gene locus.

24. The method of claim 23, wherein the heterologous sequence is integrated into exon 1, 2, or 3 of the TRAC gene locus.

25. The method of any one of claims 17-24, wherein the transgene encodes FOXP3, Helios, or ThPOK.

26. The method of claim 25, wherein the transgene comprises a coding sequence for FOXP3 and a coding sequence of ThPOK, wherein these two coding sequences are in-frame and are separated by an in-frame coding sequence for a self-cleaving peptide.

27. The method of any one of the preceding claims, wherein the starting population of cells are human cells.

28. The method of claim 27, wherein the starting population of cells comprise a null mutation in a gene selected from
a Class II major histocompatibility complex transactivator (CIITA) gene,
an HLA Class I or II gene,
a transporter associated with antigen processing,
a minor histocompatibility antigen gene, and
a β2 microglobulin (B2M) gene.

29. The method of any one of the preceding claims, wherein the starting population of cells comprise a suicide gene optionally selected from an HSV-TK gene, a cytosine deaminase gene, a nitroreductase gene, a cytochrome P450 gene, or a caspase-9 gene.

30. A population of cells enriched for CD4 single positive cells obtained by the method of any one of claims 1-29.

31. A population of cells enriched for CD4⁺ Teff cells obtained by the method of any one of claims 1-7 and 12-29.

32. A method of treating cancer, an infectious disease, an allergy, asthma, or an autoimmune or inflammatory disease in a patient in need thereof, comprising administering the population of cells of claim 31 to the patient.

33. Use of the population of cells of claim 31 for the manufacture of a medicament for treating cancer, an infectious disease, an allergy, asthma, or an autoimmune or inflammatory disease in a patient in need thereof.

34. A population of cells of claim 31 for use in treating cancer, an infectious disease, an allergy, asthma, or an autoimmune or inflammatory disease in a patient in need thereof.

35. A population of cells enriched for CD4⁺ Treg cells obtained by the method of any one of claims 1-6 and 8-29.

36. A method of treating a patient in need of immunosuppression, comprising administering the population of cells of claim 35 to the patient.

37. Use of the population of cells of claim 35 for the manufacture of a medicament for treating a patient in need of immunosuppression.

38. A population of cells of claim 35 for use in treating a patient in need of immunosuppression.

39. The method, use, or population of cells for use of any one of claims 36-38, wherein the patient has an autoimmune disease, or has received or will receive tissue transplantation.

40. A pharmaceutical composition comprising the population of cells of claim 30, 31, or 35 and a pharmaceutically acceptable carrier.
